(19) United States
(12) Patent Application Publication
(10) Pub. No.: US 2024/0240191 A1
(43) Pub. Date: Jul. 18, 2024

(54) RNA APTAMERS SPECIFIC FOR A-SYNUCLEIN PROTEIN FIBER CONFORMERS

(71) Applicants: COMMISSARIAT A L'ENERGIE ATOMIQUE ET AUX ENERGIES ALTERNATIVES, Paris (FR); CENTRE NATIONAL DE LA RECHERCHE SCIENTIFIQUE (CNRS), Paris (FR)

(72) Inventors: Alix BOUVIER-MULLER, FONTENAY AUX ROSES (FR); Frederic DUCONGE, FONTENAY AUX ROSES (FR); Luc BOUSSET, FONTENAY AUX ROSES (FR); Ronald MELKI, FONTENAY AUX ROSES (FR)

(21) Appl. No.: 18/564,121

(22) PCT Filed: May 25, 2022
(86) PCT No.: PCT/FR2022/050993

§ 371 Date: Nov. 27, 2023

(30) Foreign Application Priority Data
May 27, 2021 (FR) FR2105529

(51) Int. Cl. C12N 15/115; G01N 33/53; G01N 33/68

(52) U.S. Cl. CPC C12N 15/115; G01N 33/5308; G01N 33/6896; C12N 2310/16; G01N 2800/28

(57) ABSTRACT

The present invention relates to an aptamer characterized in that it has the ability to distinguish conformers of F-type α-Syn fibres of the α-Syn (α-Syn) protein from conformers of R-type α-Syn fibres, and in that it comprises a sequence specific for modified ribonucleic acid (RNA) having at least 85% identity with a sequence chosen from SEQ ID NO: 1, SEQ ID NO: 2, SEQ ID NO: 3, SEQ ID NO: 4, SEQ ID NO: 5, SEQ ID NO: 6 and SEQ ID NO: 7, preferably chosen from SEQ ID NO: 1 and SEQ ID NO: 2. The present invention also relates to a composition or a kit comprising at least one of these aptamers, and also to the uses thereof in vitro. The present invention also relates to a method for diagnosing synucleinopathies, as well as to a method for stratification, monitoring, prognosis and evaluation of the efficacy of a synucleinopathy treatment, comprising the use of at least one aptamer and/or a composition and/or a kit mentioned above.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Feb. 22, 2024, is named 18564121_SL_022224.txt and is 16,963 bytes in size.

### FIELD OF THE INVENTION

The present invention relates to the fields of aptamers and neurodegenerative diseases, in particular synucleinopathies.

The present invention relates to an aptamer characterized in that it has the ability to distinguish conformers of F-type α-Syn fibers of the α-Synuclein (α-Syn) protein from conformers of R-type α-Syn fibers, and in that it comprises a sequence specific for modified ribonucleic acid (modified RNA) having at least 85% identity with a sequence chosen from SEQ ID NO: 1, SEQ ID NO: 2, SEQ ID NO: 3, SEQ ID NO: 4, SEQ ID NO: 5, SEQ ID NO: 6, and SEQ ID NO: 7, preferably chosen from SEQ ID NO: 1 and SEQ ID NO: 2.

The present invention further relates to a composition or a kit comprising at least one of these aptamers, and also to the uses thereof. The present invention also relates to a method for diagnosing neurodegenerative diseases as well as to a method for stratification, monitoring, prognosis and evaluation of the efficacy of a synucleinopathy treatment, comprising the use of at least one aptamer and/or a composition and/or a kit mentioned above.

### STATE OF THE ART

The diagnostic of neurodegenerative diseases, such as Alzheimer's, Parkinson's, Huntington's, Creutzfeldt-Jakob's diseases or dementia with Lewy bodies, is based on the observation of cognitive, motor and sensory disorders experienced by patients. However, this diagnostic is difficult to make because the symptoms of these different diseases are very similar, especially in the early stages of the disease. Often, a definitive diagnostic can only be made when the disease has reached an advanced stage and the patient suffers from the most severe forms of symptoms (Gómez-Río and al., 2016). It is therefore essential to develop new diagnostic methods to reliably distinguish between different neurodegenerative diseases (NDs). In addition, better diagnostic would provide patients with more reliable monitoring and prognosis of the evolution of the disease, and better evaluate the efficacy of the treatments administered.

In addition, the motor symptoms related to these diseases often appear very late in the process, when the neuronal damage is already very significant. For example, it is estimated that when the motor symptoms of Parkinson's disease appear, 50% of the dopaminergic neurons in the substantia nigra are already dead (Cheng and al., 2010). Being able to detect the onset of NDs before the appearance of motor symptoms is therefore essential, because this would allow treatments to be started earlier, when the neurodegeneration phenomenon has not yet become too widespread.

Two types of markers have been particularly studied to diagnose NDs: genetic and biochemical biomarkers.

Genetic biomarkers can be alleles or mutations in the genome that have been identified as predisposing to disease. This type of marker thus allows to identify an “at risk” population, which has a higher probability of developing NDs.

Biochemical markers are biomolecules whose presence and/or amount is correlated with the evolution of the pathology. For example, Parkinson's disease is marked histologically by the accumulation of alpha-Synuclein (or α-Synuclein or α-Syn) within Lewy bodies (Katsuno and al., 2018). Biochemical markers are therefore considered as marks of the appearance and development of the disease while genetic biomarkers are rather predisposing factors for developing the disease. The identification of biochemical markers can thus allow to detect the appearance of a disease early, to make more precise diagnoses, to follow the evolution of the disease more easily, and also to precisely evaluate the efficacy of therapy. Currently, numerous efforts are therefore at work to identify such markers which would be present in the tissues or biological fluids of patients (blood, serum, cerebrospinal fluid, etc.) and would consist of distinctive signatures of each disease (Agrawal and Biswas, 2015; Beach, 2017). The accumulation of protein aggregates in the central nervous system is a common feature of several progressive neurodegenerative disorders (for example Alzheimer's, Parkinson's, Huntington's and Creutzfeldt-Jakob diseases). Three-dimensional misfolding of certain proteins can increase their tendency to bind together and transmit this misfolding to each other. Aggregated forms of these proteins then gradually accumulate and can interfere with the normal function of neurons, which can gradually lead to their death. Among the proteins that can form this type of aggregates, mention can be made of the α-Synuclein protein, the Tau protein, Huntingtin, the beta-amyloid peptide or the PrP protein.

It was demonstrated that the pathological three-dimensional conformation of these proteins could be transmissible not only between cells but also from one individual to another. This allowed to demonstrate that certain proteins, called “Prions”, could be infectious agents in the same way as viruses, bacteria and parasites. Although transmission from one individual to another has mainly been demonstrated for the PrP protein, recent studies have demonstrated that other proteins involved in neurodegenerative diseases could have comparable properties. Among these proteins, mention can also be made of the α-Synuclein protein, the Tau protein, Huntingtin or the beta-amyloid peptide. α-Synuclein (α-Syn) fibers are found within protein aggregates in the brain of patients suffering from certain NDs grouped under the name “synucleinopathies”. These diseases comprise, among others: Parkinson's disease (PD), dementia with Lewy bodies (DLB), and multiple system atrophy (MSA). α-Syn fibers are composed of thousands of misfolded protein repeats. They are formed by successive recruitment of proteins, according to a propagation mechanism where a misfolded α-Syn protein transmits its erroneous conformation when it binds to another protein. Recently, several forms of α-Syn fibers with distinct structural conformations were isolated and it has been shown that they could induce different pathologies (Peelaerts and al., 2015; Rey and al., 2019). These different conformations are called conformers. Among these conformers, mention can be made of conformers of α-Syn fiber of type F, R, 91 and 65 (Bousset and al., 2013; Makky and al., 2016). Conformers of α-Syn fibers of type F, 65 and 91 have a cylindrical shape, while the conformers of R-type fiber have a flat ribbon shape, 91 and 65 fibers are twisted but with different helical pitches. Analyzes by solid-state NMR and atomic force microscopy allowed to clarify the structural differences between these four fiber conformers. Thus these fiber conformers are 1 to 2 μm long on average, with a width of the order of 15 to 20 nm, the conformers of F-type α-Syn fibers being the narrowest, and the 65 fiber conformers being the widest. Their height is of the order of 5 to 7 nm, with the conformers of R-type α-Syn fibers being the lowest and the fiber conformers 65 the tallest. Finally, the 65 and 91 fiber conformers have periodic variations in height, unlike the conformers of F and R type α-Syn fiber. Other analyzes also demonstrated that the four fiber conformers had different mechanical properties. Thus, the conformers of F-type α-Syn fibers are the stiffest, with a bending strength measurement four times greater than the R fiber conformers and twice greater than the 65 and 91 fiber conformers. The α-Syn fiber conformers can also be distinguished from each other by analysis by proteolysis using proteinase K. Their structural difference results in degradation profiles which are different from one conformer to another. These profiles are comparable to barcodes (or fingerprints) specific to each conformer (see an example of a degradation profile in FIG. 1 (Landureau and al., 2021). Additionally, the whole form of the protein in the conformers of F-type α-Syn fibers is more resistant to proteolysis than in the R-type conformer (Fenyi and al., 2021).

Different studies suggest that these different α-Syn fiber conformers could be specific markers of different synucleinopathies. A rat animal model allowed to demonstrate that the injection of F or R type fiber conformers into the animal brain led to two different phenotypes. It was in particular shown that the conformers of F-type α-Syn fibers caused a greater motor deficit in this model, while the conformers of R-type α-Syn fibers caused a greater number of depositions of Lewy body and neurite types (Peelaerts and al., 2015). More recent studies have shown that fiber conformers obtained from PD or SMA patients resembled the R-type fiber conformers while fibers from patients with DLB resembled the F-type fiber conformers (Van der Perren and al., 2020).

It is therefore essential to develop methods allowing efficient, specific, sensitive and reliable detection of these markers.

Several antibodies have been developed to be able to quantify α-Syn in plasma or cerebrospinal fluid (CSF) (WO2007/011907; Perrin and al., 2003; Vaikath and al., 2019). However, most of these antibodies are not specific for a particular conformation of the protein. As a result, they allow to measure the protein but are not useful from a diagnostic point of view because they do not allow to determine the amount of protein which is in aggregate form. New antibodies have been developed to specifically recognize the aggregated form of the protein compared to the monomeric form (Vaikath and al., 2015). However, these antibodies are not specific for a particular fiber type. On the other hand, it is always difficult and expensive to use multiple antibodies to quantify multiple biomarkers at the same time.

In this context, aptamers offer advantageous possibilities. Aptamers are nucleic acid structures with properties comparable to antibodies. They are generally obtained by a process of directed molecular evolution called SELEX (Systematic Evolution of Ligands by EXponential enrichment). Since the discovery of SELEX in the 1990s, the use of aptamers has been experimentally validated for numerous applications (diagnostic, purification, therapy, etc.). In addition, an anti-VEGF aptamer (Macugeno) has already received marketing authorization for the treatment of macular degeneration of the eye.

A few studies have led to the identification of DNA aptamers recognizing α-Syn. A DNA aptamer (named M5-15) was selected against the monomeric form of α-Syn. However, this aptamer also binds nonspecifically to oligomeric and fibrillar forms of α-Syn (Tsukakoshi and al., 2010). A second study allowed to obtain a second DNA aptamer (T-S0508) capable of discriminating the oligomeric form from the monomeric and fibrillar forms of α-Syn. However, additional tests demonstrated that this aptamer also recognized the oligomeric form of Amyloid-β with an affinity of the same order of magnitude (Kd of approximately 100 nM; Tsukakoshi and al., 2012). Aptamers F5R1 and F5R2, which are also in DNA chemistry, were selected in 2019 by Zheng and colleagues. They recognize α-Syn with kd of 2.4 and 3.07 nM respectively (Zheng and al., 2018; Ren and al., 2019). Other aptamers in DNA chemistry have also been selected by Derosa and co-workers (WO201979887). None of these different aptamers have been evaluated for their ability to discriminate different α-Syn fiber conformers.

In view of the importance of α-Syn fibers and, in particular, of conformers of F and R type α-Syn fibers, in the diagnostic of different neurodegenerative diseases, it is essential to develop molecules capable of distinguishing the different α-Syn fiber conformers, in particular conformers of F and R type α-Syn fibers.

The present invention allows to meet this need.

### DISCLOSURE OF THE INVENTION

In the context of the present invention, the inventors have developed ribonucleic acid (RNA) aptamers capable of specifically recognizing certain conformers of α-Syn fibers. The inventors have in fact selected and isolated RNA aptamers having different affinities for different α-Syn fiber conformers.

The inventors have in particular shown that, surprisingly, the aptamers developed are capable of distinguishing the conformers of F-type α-Syn fibers from conformers of R-type α-Syn fibers, unlike the DNA aptamers against α-Syn described in the prior art.

The data show in particular that the aptamers developed by the inventors have a strong affinity for the conformer of F-type α-Syn fibers (dissociation constant K_{d} between 5 and 10 nM). Unexpectedly, these aptamers, on the other hand, have a very low or even zero affinity for the conformer of R-type α-Syn fibers. The inventors have also demonstrated that these aptamers recognize with an affinity at least 10 times lower the α-Syn protein in native form (monomeric, non-fibrillar form). Remarkably, the inventors have developed a method using a mixture of these aptamers which allows to effectively discriminate these fiber conformers by high-throughput sequencing, applicable to patient samples.

The data show that these aptamers are tools for specific and sensitive detection of different α-Syn fibers. The present invention therefore provides both effective and reliable methods for diagnosing neurodegenerative diseases, methods for screening molecules but also tools for research in the field of neurodegenerative diseases.

The present invention therefore relates to an aptamer characterized in that it has the ability to distinguish conformers of F-type α-Syn fibers of the α-Syn (α-Syn) protein from conformers of R-type α-Syn fibers, and in that it comprises a sequence specific for modified ribonucleic acid (RNA) having at least 85% identity with a sequence chosen from SEQ ID NO: 1, SEQ ID NO: 2, SEQ ID NO: 3, SEQ ID NO: 4, SEQ ID NO: 5, SEQ ID NO: 6, and SEQ ID NO: 7, preferably chosen from SEQ ID NO: 1 and SEQ ID NO: 2. According to a preferred embodiment, the RNA was modified in order to increase its resistance to RNA nucleases, preferably by modifying the riboses of the pyrimidines of the aptamer so that they carry a fluorine atom on carbon in position 2′.

The present invention further relates to a composition or a kit comprising at least one of these aptamers, and also to the uses thereof. The present invention also relates to a method for diagnosing neurodegenerative diseases as well as to a method for stratification, monitoring, prognosis and evaluation of the efficacy of a synucleinopathy treatment, comprising the use of at least one aptamer and/or a composition and/or a kit mentioned above.

### DETAILED DESCRIPTION OF THE INVENTION

### Summary of the Invention

The present invention relates to an aptamer characterized in that it has the ability to distinguish conformers of F-type α-Syn fibers of the α-Syn protein (α-Syn) from conformers of R-type α-Syn fibers, and in that it comprises a sequence specific for modified ribonucleic acid (RNA) having at least 85% identity with a sequence chosen from SEQ ID NO: 1, SEQ ID NO: 2, SEQ ID NO: 3, SEQ ID NO: 4, SEQ ID NO: 5, SEQ ID NO: 6, and SEQ ID NO: 7, preferably chosen from SEQ ID NO: 1 and SEQ ID NO: 2.

According to one embodiment of the aptamer according to the invention, the dissociation constant K_{d(F)} measured for the conformers of F-type α-Syn fibers is:
a) lower than the dissociation constant K_{d(R)} measured for the conformers of R-type α-Syn fibers, preferably lower by at least 10 times;
b) lower than the dissociation constant K_{d(Mono)} measured for α-Syn monomers, preferably lower by at least 2 times;
c) lower than the dissociation constant K_{d(Random)} of a random aptamer measured for the conformers of F-type α-Syn fibers, preferably lower by at least 2 times;
d) lower than the dissociation constant K_{d(R)} measured for the conformers of R-type α-Syn fibers, preferably lower by at least 10 times; and lower than the dissociation constant K_{d(Mono)} measured for α-Syn monomers, preferably lower by at least 2 times;
e) lower than the dissociation constant K_{d(R)} measured for the conformers of R-type α-Syn fibers, preferably lower by at least 10 times; and lower than the dissociation constant K_{d(Random)} of a random aptamer measured for the conformers of F-type α-Syn fibers, preferably lower by at least 2 times; or
f) lower than the dissociation constant K_{d(R)} measured for the conformers of R-type α-Syn fibers, preferably lower by at least 10 times; lower than the dissociation constant K_{d(Mono)} measured for α-Syn monomers, preferably lower by at least 2 times; and lower than the dissociation constant K_{d(Random)} of a random aptamer measured for the conformers of F-type α-Syn fibers, preferably lower by at least 2 times.

According to one embodiment of the aptamer according to the invention, at least one dissociation constant K_{d} is as follows:
a) the dissociation constant K_{d(F)} measured for the conformers of F-type α-Syn fibers is less than 15 nM, preferably less than 10 nM; and/or
b) the dissociation constant K_{d(R)} measured for the conformers of R-type α-Syn fibers is greater than 100 nM, preferably greater than 150 nM.

The present invention further relates to an aptamer as above, further comprising:
i. in 5′ of the specific sequence, a modified RNA primer sequence having at least 85% identity, preferably at least 90% identity, preferably at least 95% identity, with a sequence chosen from SEQ ID NO: 29 and SEQ ID NO: 30, preferably located at the end 5′ of the specific sequence; and/or
ii. in 3′ of the specific sequence, a modified RNA primer sequence having at least 85% identity, preferably at least 90% identity, preferably at least 95% identity, with a sequence chosen from SEQ ID NO: 31, SEQ ID NO: 32 and SEQ ID NO: 33, preferably located at the end 3′ of the specific sequence.

The present invention further relates to an aptamer as above, comprising a modified RNA sequence having at least 85% identity with a sequence chosen from SEQ ID NO: 34, SEQ ID NO: 35, SEQ ID NO: 36, SEQ ID NO: 37, SEQ ID NO: 38, SEQ ID NO: 39, and SEQ ID NO: 40, preferably chosen from SEQ ID NO: 34 and SEQ ID NO: 35.

The present invention also relates to a kit comprising at least one aptamer according to the invention.

According to one embodiment of the kit according to the invention, the kit further comprises at least one additional aptamer chosen from aptamers comprising a sequence specific for modified RNA having at least 85% identity with a sequence chosen from SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17, SEQ ID NO: SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28;
the kit preferably further comprising an aptamer comprising a modified RNA random sequence.

According to one embodiment of the kit according to the invention, at least one additional aptamer further comprises:
i. in 5′ of the specific sequence, a modified RNA primer sequence having at least 85% identity, preferably at least 90% identity, preferably at least 95% identity, with a sequence chosen from SEQ ID NO: 29 and SEQ ID NO: 30, preferably located at the end 5′ of the specific sequence; and/or
ii. in 3′ of the specific sequence, a modified RNA primer sequence having at least 85% identity, preferably at least 90% identity, preferably at least 95% identity, with a sequence chosen from SEQ ID NO: 31, SEQ ID NO: 32 and SEQ ID NO: 33, preferably located at the end 3′ of the specific sequence.

The present invention also relates to a kit as above, in which at least one additional aptamer is chosen from aptamers comprising a modified RNA sequence having at least 85% identity with a sequence chosen from SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48 and SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, and SEQ ID NO: 60.

According to one embodiment of the kit according to the invention, the kit comprises at least the following aptamers:
aptamer comprising a sequence specific for modified RNA having at least 85% identity with SEQ ID NO: 1,
aptamer comprising a sequence specific for modified RNA having at least 85% identity with SEQ ID NO: 2,
aptamer comprising a sequence specific for modified RNA having at least 85% identity with SEQ ID NO: 3,
aptamer comprising a sequence specific for modified RNA having at least 85% identity with SEQ ID NO: 8,
aptamer comprising a sequence specific for modified RNA having at least 85% identity with SEQ ID NO: 9,
aptamer comprising a sequence specific for modified RNA having at least 85% identity with SEQ ID NO: 10,
aptamer comprising a sequence specific for modified RNA having at least 85% identity with SEQ ID NO: 11,
aptamer comprising a sequence specific for modified RNA having at least 85% identity with SEQ ID NO: 12,
aptamer comprising a sequence specific for modified RNA having at least 85% identity with SEQ ID NO: 13,
aptamer comprising a sequence specific for modified RNA having at least 85% identity with SEQ ID NO: 14,
aptamer comprising a sequence specific for modified RNA having at least 85% identity with SEQ ID NO: 16,
aptamer comprising a sequence specific for modified RNA having at least 85% identity with SEQ ID NO: 17, and
aptamer comprising a sequence specific for modified RNA having at least 85% identity with SEQ ID NO: 18;
the kit preferably further comprising an aptamer comprising a modified RNA random sequence.

The kit according to the invention is preferably characterized in that, when the kit comprises several aptamers, said aptamers are:
a) all in one composition, or
b) distributed in several distinct compositions in separate containers, including the case where each of the aptamers is in a distinct composition located in a separate container.

The present invention further relates to an aptamer as above or a kit as above, in which the RNA of the aptamer or of all the aptamers of the kit was modified in order to increase its resistance to RNA nucleases, preferably in which the riboses of the pyrimidines of the aptamer or of all the aptamers of the kit carry a fluorine atom on the carbon in the position 2′.

The present invention also relates to an in vitro use of at least one aptamer according to the invention, a use of at least one kit according to the invention, or a use of any combination thereof, for:
a) detecting the presence or absence of at least one conformer of F-type α-Syn fibers in a biological sample;
b) determining the amount of a conformer of F-type α-Syn fibers in a biological sample; c) establishing a molecular fingerprint of α-Syn fiber conformers, preferably F and R type α-Syn fibers of α-Syn, in a biological sample;
d) screening compounds/molecules capable of detecting and/or recognizing a conformer of F-type α-Syn fibers, preferably screening compounds/molecules capable of discriminating the conformers of F-type α-Syn fibers from conformers of R-type α-Syn fibers; or
e) any combination of a) to d).

The present invention also relates to an in vitro method for diagnosing a synucleinopathy in a subject having at least one symptom of neurodegenerative disease, comprising:
a) contacting a biological sample of the subject with at least one aptamer according to the invention, with at least one kit according to the invention, or with any combination thereof;
b) detecting the presence or absence of at least one conformer of F-type α-Syn fibers, quantifying the conformers of F-type α-Syn fibers, establishing a molecular fingerprint of α-Syn fiber conformers (preferably F and R type α-Syn fibers of α-Syn), or any combination thereof, in the subject's biological sample; and
c) diagnosing the presence or absence of a synucleinopathy in the subject based on the result of step b).

The present invention also relates to an in vitro method for stratification of a synucleinopathy, prognosis of a synucleinopathy, monitoring of a synucleinopathy, or evaluation of the efficacy of a synucleinopathy treatment in a subject suffering from a synucleinopathy, comprising:
a) contacting a biological sample of the subject with at least one aptamer according to the invention, with at least one kit according to the invention, or with any combination thereof;
b) detecting the presence or absence of at least one conformer of F-type α-Syn fibers, quantifying the conformers of F-type α-Syn fibers, establishing a molecular fingerprint of α-Syn fiber conformers (preferably F and R type α-Syn fibers of α-Syn), or any combination thereof, in the subject's biological sample; and
c) stratifying the synucleinopathy, prognosing the synucleinopathy, monitoring the synucleinopathy, evaluating the efficacy of the treatment of the synucleinopathy, in the subject, based on the result of step b);
wherein synucleinopathy is preferably selected from Parkinson's disease (PD), dementia with Lewy bodies (DLB) and multiple system atrophy (MSA), more preferably synucleinopathy is DLB.

### Definitions

“α-Synuclein” or “α-Syn” or “α-syn” or “alpha-Synuclein” or “α-Synuclein” means a phosphoprotein of the synuclein family which is abundant in the human brain. Small amounts are also found in the heart, muscles and other tissues. In the brain, α-Syn is found primarily at the ends of nerve cells (neurons) in presynaptic terminals. The reference protein sequence for the human α-Synuclein protein is the NCBI sequence referenced under the number P37840.1.

In the physiological state, the α-Syn protein exists in two forms. It can be soluble or bound to a membrane. In the soluble state in the cytosol, α-Syn has a disordered structure (Fauvet and al., 2012). In the presence of a lipid membrane, the N-terminal part of α-Syn adopts an α-helical structure which allows it to be embedded within lipid membranes (Eliezer and al., 2001). When bound to a large diameter membrane (at least 100 nm), α-Syn adopts the shape of a large elongated helix (Trexler and Rhoades, 2009). When, on the contrary, it interacts with small vesicles (and therefore with high curvature), α-Syn adopts a structure composed of two small a helixes (Chandra and al., 2003).

In the event of abnormal folding, α-Syn can also adopt conformations called “pathological” conformations, rich in β-sheets. These pathological conformations have a strong propensity to form fibrillar aggregates (called “fibrillar α-Syn” or “α-Syn fibers”) which are found in the form of intracellular depositions in synucleinopathies (El-Agnaf and al., 1998a). “Fibrillar α-Syn” or “α-Syn fiber” or “α-Syn fibrillar aggregate” or “α-Syn aggregate”, means a fiber (or an aggregate, or an assembly) composed of numerous misfolded α-Syn protein repeats (in particular 25 to several hundred misfolded α-Syn protein repeats). It is therefore an assembly of several copies (from 25 to several hundreds) of misfolded α-Syn proteins (having abnormal/pathological/non-native folding). These α-Syn fibers are formed by successive recruitment of α-Syn proteins, according to a propagation mechanism where a misfolded α-Syn protein transmits its erroneous conformation when it binds to another protein. These insoluble fibers can be found in the Lewy bodies characteristic of certain pathologies known as synucleinopathies, such as Parkinson's disease (PD), dementia with Lewy bodies (DLB), and multiple system atrophy (MSA).

Several forms of α-Syn fibers with distinct structural conformations have been isolated and it has been demonstrated that they could induce different synucleinopathies. These different conformations are called α-Syn fiber conformers.

The α-Syn protein may undergo one or more post-translational modifications, such as the addition of a functional group (for example chosen from acetylation, alkylation, biotinylation, carboxylation, glutamylation, glycylation, glycosylation, hydroxylation, isoprenylation, lipoylation, phosphopantetheinylation, phosphorylation, ribosylation, sulfation, selenation, amidation, etc., and any combination thereof), the addition of peptide or protein groups (provided for the latter that the added protein is not an α-Syn protein; the addition of peptide groups or proteins is for example chosen from ubiquitination, neddylation, sumoylation, urmylation, etc., and any combination thereof), changing the chemical nature of an amino acid (for example chosen from citrullination, deamidation, etc., and any combination thereof), a structural change (for example chosen from disulfide bridge formation, etc.), and any combination thereof.

“α-Syn conformer” or “α-Syn fiber conformer” means a specific structural/spatial configuration (conformation) formed by an aggregate of α-Syn proteins having an abnormal folding (that is to say a specific structural configuration adopted by the fibrillar α-Syn). Different α-Syn fiber conformers have been identified such as conformers of α-Syn fibers of type F, R, 65 and 91. Conformers of α-Syn fibers of type F, 65 and 91 have a cylindrical shape, while the type-R fiber conformers have a flat ribbon shape, 91 and 65 fibers are twisted but with different helical pitches. Within these fibers, at least one copy of the α-Syn protein may comprise one or more post-translational modifications as defined above.

“F fiber” or “conformer of F-type α-Syn fibers” or “conformer of α-Syn F-type fibers” mean an aggregate of α-Syn proteins which is in a cylindrical shape, producing 9 bands during their proteolysis carried out by proteinase K (example in FIG. 1 (Landureau and al., 2021)) and whose bending stiffness is approximately 5.8 10⁻²⁶ N·m².

“R fiber” or “conformers of R-type α-Syn fibers” or “conformers of α-Syn R-type fiber” means an aggregate of α-Syn proteins which appears in the shape of flat ribbons. Type R fibers are more flattened than type F fibers. They produce 3 bands during their proteolysis carried out by proteinase K (example in FIG. 1 (Landureau and al., 2021)) and have a bending stiffness of approximately 1.4 10⁻²⁶ N·m².

“65 Fiber” or “65 fiber conformer” or “65 α-Syn fiber conformer” means an aggregate of α-Syn proteins which appears in the shape of tight twists. They have a bending stiffness of approximately 2.7 10⁻²⁶ N·m².

“91 Fiber” or “91 fiber conformer” or “91 α-Syn fiber conformer” means an aggregate of α-Syn proteins which appears in the shape of loose twists. They produce 7 bands during their proteolysis by proteinase K (example in FIG. 1 (Landureau and al., 2021)) and have a bending stiffness of approximately 2.4 10⁻²⁶ N·m².

These different types of fibers can be distinguished using the different molecular and structural analysis techniques known to the person skilled in the art, such as solid-state NMR analysis, atomic force microscopy, electron microscopy, controlled proteolysis, the diffraction of X-rays by the fibers, the fixation of the ligand such as Thioflavin T or antibodies, etc. These techniques have in particular allowed to show that the F, R, 65 and 91 fiber conformers are 1 to 2 μm long on average, with a width of around 15 to 20 nm, the conformers of F-type α-Syn fiber being the narrowest, and the 65 fiber conformers the widest. Their height is of the order of 5 to 7 nm, the conformers of R-type α-Syn fibers being the lowest and the 65 the highest. Finally, the 65 and 91 fiber conformers have periodic variations in height, unlike the conformers of F and R type α-Syn fibers. Alternatively or in combination with molecular and structural analysis techniques, these different types of fiber conformers can be distinguished by their mechanical properties, using different mechanical analysis techniques known to the person skilled in the art, such as atomic force microscopy. These mechanical analyzes in particular allowed to demonstrate that the four types of fibers had different mechanical properties. Thus, the conformers of F-type α-Syn fibers are the stiffest, with a bending strength measurement four times greater than the R fiber conformer and twice greater than the 65 and 91 fiber conformers. α-Syn fiber conformers can also be distinguished from each other (in particular α-Syn fiber conformers can be distinguished from other types of α-Syn fiber conformers) by their protease degradation profile. Their structural difference results in degradation profiles that are different from one conformer to another. These profiles are comparable to barcodes (or fingerprints) specific to each conformer (see an example of a degradation profile in FIG. 1). Additionally, the whole form of the protein in the conformers of F-type α-Syn fibers is more resistant to proteolysis than in the R-type conformer (Fenyi and al., 2021).

Within these fibers, at least one copy of the α-Syn protein may comprise one or more post-translational modifications as defined above.

“α-Syn oligomer” or “α-Syn in oligomeric form” means an assembly comprising several copies (between 2 and 24) of the protein of the α-Syn protein, presented in the form of a chain (that is to say a sequence of α-Syn proteins bound/associated with each other, comprising between 2 and 24 α-Syn proteins). An α-Syn oligomer is distinguished from an α-Syn fiber in that it comprises a lower number of copies of α-Syn (Pieri and al., 2016). An α-Syn oligomer therefore comprises from 2 to around twenty copies of the α-Syn protein. An α-Syn oligomer is not an α-Syn fiber within the meaning of the present invention, since it comprises less than 25 α-Syn monomers.

“α-Syn monomer” or “α-Syn in monomeric form” means a molecule of the α-Syn protein present in a free form, that is to say which is not bound/associated to another molecule of the α-Syn protein. An α-Syn monomer therefore comprises a single copy of the α-Syn protein. An α-Syn monomer is therefore distinguished from oligomers and aggregates/fibers of the α-Syn protein. The single copy of the α-Syn protein in the monomer may nevertheless comprise one or more post-translational modifications, as defined above.

“Aptamer” means an oligonucleotide (that is to say a segment of a nucleic acid chain) which adopts a three-dimensional structure giving it the ability to bind specifically to a given ligand (the ligand is called “target”), particularly of a protein nature. An aptamer is said to bind specifically to a target when it has essentially no affinity for a compound structurally unrelated to the target. Preferably, in the case of a protein target, a protein compound is said to have no structural relationship with the target according to the invention, when the sequence identity between the target and the compound is less than 60%, preferably less than 70%, preferably even less than 80%. Preferably, according to the invention, an aptamer is said to have essentially no affinity for a compound according to the invention, in particular when the dissociation constant of the aptamer with respect to the compound is greater than 10⁻⁶ mol/l, preferably greater than 10⁻⁷ mol/l. The dissociation constant can in particular be determined, under standard conditions, using the Scatchard and Lineweaver Burk representations well known to the person skilled in the art.

Aptamers generally comprise from a few nucleotides to a few dozen nucleotides, for example from 15 to 100 nucleotides (preferably from 20 to 90 nucleotides, more preferably from 30 to 80 nucleotides, more preferably from 40 to 70 nucleotides, more preferably from 50 to 60 nucleotides). Aptamers are mainly manufactured synthetically, using techniques known to the person skilled in the art (such as chemical or enzymatic synthesis). Aptamers are generally selected/identified by a process of directed molecular evolution called SELEX (Systematic Evolution of Ligands by EXponential enrichment).

The aptamer may comprise at least one modified nucleotide (that is to say a nucleotide which is not a nucleotide of a natural DNA or RNA). These modified nucleotides can in particular be used to increase the resistance of the aptamer to degradation by nucleases. This is particularly advantageous for RNA aptamers, which are generally more sensitive to nucleases than DNA aptamers. An RNA comprising at least one modified nucleotide is called modified RNA.

The aptamer may also comprise at least one additional group, in addition to the nucleotides constituting its nucleic acid sequence. Thus, the nucleic acid of the aptamer can be bound to at least one additional group.

“Modified RNA” means an RNA including at least one modified nucleotide. A modified RNA may in particular be an RNA in which the backbone of the nucleic acid is modified, in whole or in part, in particular to make it resistant to hydrolytic degradation, in particular due to the action of nucleases. RNA can be modified in its entirety (that is to say each nucleotide which constitutes it is modified) or in part (that is to say only part of the nucleotides which constitute it is modified). When the RNA is partially modified, it is possible to choose to modify all or part of the purines and/or all or part of the pyrimidines.

The modifications of an RNA (and/or a nucleotide) are well known to the person skilled in the art and can in particular be chosen from: the modification of the OH function on the carbon in the position 2′ of the ribose by methylation; the substitution of the OH function on the carbon in the position 2′ of the ribose by an O-Methoxyethyl group; the substitution of the OH function on the carbon in the position 2′ of the ribose by an amino group; the substitution of the OH function on the carbon in the position 2′ of the ribose by a halogen (in particular by fluorine); the replacement of the phosphodiester (PO) by a phosphorothioate (PS) group (therefore this is referred to as a phosphorothioate skeleton); the use of a Locked Nucleic Acid (LNA) type structure, that is to say the formation of a methylene bridge in order to lock the ribose in the C3′-endo (N-type) conformation; the use of a Peptide Nucleic Acid (PNA) type structure, that is to say the replacement of the sugar-phosphate backbone with a peptide type backbone; and any combination thereof.

“Additional group” means a chemical group of any type and nature, not forming part of the nucleic sequence of the aptamer. The additional group may in particular be chosen from: a radioisotope, an organic molecule comprising 100 carbon atoms at most, a nanoparticle, a protein (in particular a glycoprotein), a carbohydrate, a lipid, a polynucleotide, and any combination thereof. The additional group is preferably chosen from: a detectable marker, a pharmacological compound, a compound capable of modifying the pharmacokinetic characteristics of a nucleic acid to which it is bound (such as polyethylene glycol (PEG)), and any combination thereof.

The detectable marker can be of any type, it can in particular be a fluorophore (for example fluorescein or luciferase), a radioisotope (in particular adapted for scintigraphy, for example ^{99m}Tc), a label recognizable by an antibody (for example c-Myc protein or a polyhistidine tag), an affinity tag (for example biotin), an enzyme (for example horseradish peroxidase), a contrast agent, etc.

The pharmacological compound can also be of any type. It may in particular be an anticancer chemotherapy agent (such as a cytostatic or cytological agent), an antibody, a toxin, a hormone, an enzyme, an antiviral compound, an antibiotic compound, an antifungal compound, an antibacterial compound, etc.

“Specific/target sequence of an aptamer” or “specific/target sequence of an aptamer” means the part (section/region/portion) of the sequence of an aptamer which is specific to the ligand (target) of the aptamer, that is to say the sequence which is specific to a specific aptamer. The specific sequence of an aptamer varies from one aptamer to another (it is therefore a variable sequence, in contrast to constant sequences which may also be present in an aptamer). The specific sequence of an aptamer is therefore distinguished from the primer sequences (or “constant” sequences, or “aspecific” sequences) likely to also be present in an aptamer.

“Primer sequence of an aptamer” or “constant sequence of an aptamer” means a part (section/region/portion) of the sequence of an aptamer which is present in all the aptamers which have been identified/selected by the same session (the same implementation) of the selection method used (such as SELEX). This is generally the sequence of a primer used for the PCR step during SELEX. An aptamer therefore generally comprises two primer sequences, one in 5′ of the specific/target sequence, and the other in 3′ of the specific/target sequence, thus allowing amplification by PCR. The primer sequences of an aptamer are therefore not specific to the aptamer.

“Random sequence” means a sequence used as a control and designed randomly by the person skilled in the art (that is to say the nucleotides which constitute it are assembled randomly). The random sequence preferably has the same length as that of the aptamer. The random sequence preferably has the same “constant” sequences (“primers”; preferably 3′ and/or 5′) as the aptamer, if applicable. In this case, the constant sequences frame the random sequence of the aptamer (the constant sequences are therefore 5′ and/or 3′ of the random sequence of the random aptamer) to form a random aptamer whose complete sequence has the same length as that of the aptamer according to the invention also having constant sequences (identical or different from those of the random aptamer, preferably identical).

“Ability to distinguish/discriminate at least two conformers of the fibrillar α-Synuclein (α-Syn) protein” means the property that a molecule (for example an aptamer) has allowing it to bind to at least one of the α-Syn fiber conformers (such as F-, R-, 91-, and 61-type fiber conformers) with significantly higher affinity (that is to say, with a lower dissociation constant) than at least one other α-Syn fiber conformer. In particular, a molecule is capable of distinguishing at least two α-Syn fiber conformers when it has a significantly stronger affinity for one α-Syn fiber conformer compared to the other α-Syn fiber conformer. In other words, a molecule is capable of distinguishing at least two α-Syn fiber conformers when it has a binding ability to an α-Syn fiber conformer significantly greater than its binding ability to at least one other α-Syn fiber conformer. For example, the molecule is capable of distinguishing at least two α-Syn fiber conformers when it has a significantly lower dissociation constant for one α-Syn fiber conformer than for at least one other α-Syn fiber conformer.

The molecule may in particular be capable of distinguishing at least the conformer of F-type α-Syn fibers of the α-Syn protein from the conformer of R-type α-Syn fibers of the α-Syn protein, if it has a significantly higher affinity for (binding ability to) the conformer of F-type α-Syn fibers than for (to the) conformer of R-type α-Syn fibers. “Ability to distinguish/discriminate the conformer of F-type α-Syn fibers of the α-Syn protein from the conformer of R-type α-Syn fibers” therefore means the property that a molecule has allowing it to bind to the conformer of F-type α-Syn fibers with significantly higher affinity (that is to say with a lower dissociation constant) than to the R conformer of R-type α-Syn fibers. For example, the molecule is capable of distinguishing the conformer of F-type α-Syn fibers from the conformer of R-type α-Syn fibers when its average (measured/calculated) dissociation constant for the conformer of F-type α-Syn fibers is significantly lower than its average (measured/calculated) dissociation constant for the conformer of R-type α-Syn fibers. In particular, a molecule is capable of distinguishing the conformer of F-type α-Syn fibers from the conformer of R-type α-Syn fibers when it is able to bind to the conformer of F-type α-Syn fibers but not to the conformer of R-type α-Syn fibers.

In particular, the molecule may be capable of further distinguishing the conformer of F-type α-Syn fibers of the α-Syn protein from the other fiber conformers of the α-Syn protein (such as the 65 fiber conformer and the 91 fiber conformer, in addition to the conformer of R-type α-Syn fibers); and/or the molecule may in particular be capable of further distinguishing the conformer of F-type α-Syn fibers of the α-Syn protein from other forms of the α-Syn protein (such as the α-Syn monomer and/or the α-Syn oligomers). The definition given above to the terms “ability to distinguish/discriminate the conformer of F-type α-Syn fibers of the α-Syn protein from the conformer of R-type α-Syn fibers” then applies, mutatis mutandis, to the ability to distinguish the conformer of F-type α-Syn fibers from the 65 α-Syn fiber conformer, to the ability to distinguish the conformer of F-type α-Syn fibers from the 91 α-Syn fiber conformer, to the ability to distinguish the conformer of F-type α-Syn fibers from the α-Syn monomer, and to the ability to distinguish the conformer of F-type α-Syn fibers from α-Syn oligomers.

“Equilibrium dissociation constant” or “K_{d}” or “Kd” means the constant which allows to evaluate the affinity between two molecules (for example between an aptamer and a fiber of the α-Syn protein). This affinity is based on the nature, geometry and number of physicochemical interactions between the two molecules (electrostatic interaction, hydrogen bonds, van der Waals interaction and hydrophobic forces). The lower the Kd value, the higher the binding affinity between the two molecules. The Kd is expressed in M (moL/L), often in nM or pM.

There are several techniques for determining/measuring dissociation constants, which are well known to the person skilled in the art, such as ELISA, gel retardation tests, filtration, chromatography, thermophoresis, “pull-down” tests, equilibrium dialysis, analytical ultracentrifugation, surface plasmon resonance (SPR), spectroscopic tests, isothermal titration calorimetry (ITC), nitrocellulose membrane filtration, etc. The equilibrium dissociation constant can in particular be determined, under standard conditions, using the Scatchard and Lineweaver Burk representations well known to the person skilled in the art.

“Neurodegenerative disease” or “ND” means a pathology whose primary cause is the death of neurons. NDs refer to a group of pathologies with very diverse clinical symptoms which have in common being slow-progressing chronic diseases characterized by dysfunction and progressive death of nerve cells (Gao and Hong, 2008). The disorders induced by this neurodegeneration can be motor, cognitive or even sensory. They worsen as the disease progresses and increasingly handicap patients. The frequency of NDs increases significantly with age. However, due to the progressive aging of the population, the number of people suffering from NDs has increased considerably in recent decades and is expected to grow steadily in the years to come (Heemels, 2016). For example, the World Health Organization currently estimates the number of people suffering from dementia worldwide at 50 million, and predicts 250 million patients by 2050 (World Alzheimer Report, 2019; Naqvi, 2017). NDs are extremely disabling pathologies which progressively lead to a loss of autonomy in patients.

The best known NDs are: Alzheimer's disease (AD), Parkinson's disease (PD), Huntington's disease and amyotrophic lateral sclerosis (ALS). NDs in particular include synucleinopathies.

“Synucleinopathy” or “α-Synucleinopathies” or “alpha-synucleinopathies” means a neurodegenerative disease characterized by the abnormal accumulation of α-Syn protein aggregates in neurons, nerve fibers or glial cells. α-Synucleinopathies are chronic and progressive pathologies which manifest by motor and cognitive disorders and behavioral changes. Synucleinopathies comprise Parkinson's disease (PD), dementia with Lewy bodies (DLB), and multiple system atrophy (MSA). These three diseases have in particular the occurrence of behavioral disorders in paradoxical sleep, dysautonomia and asymmetric parkinsonian syndrome. Synucleinopathies also comprise rarer conditions, such as the various neuroaxonal dystrophies

“Diagnostic” means the identification/determination of a disease, or the absence of a disease, in a subject. The diagnostic comprises, for example, the search for the causes (etiology) and effects (symptoms) of the disease, in particular on the basis of observations and/or measurements, carried out using different tools. In the case of neurodegenerative diseases, diagnostic tools include the observation of cognitive, motor and sensory disorders experienced by patients and the detection/quantification of genetic and/or biochemical biomarkers. Genetic biomarkers may be alleles or mutations in the genome that have been identified as predisposing to ND. This type of marker thus allows to identify an “at risk” population, which has a higher probability of developing NDs. Biochemical markers are biomolecules whose presence and/or amount is correlated with the evolution of the pathology (for example the accumulation of alpha-Synuclein for synucleinopathies).

“Stratification” means the separation/classification of subjects into subgroups by severity/seriousness of the disease. The different subgroups comprise in particular the subgroup of healthy subjects as well as different subgroups of subjects suffering from a disease, classified according to the stage of evolution/advancement of the disease. It is also possible to stratify subjects according to the type of symptoms present. The stage of development and symptoms can be determined on the basis of observations and/or measurements, carried out using different tools. In the case of neurodegenerative diseases, stratification tools comprise tools that are also used for their diagnostic.

“Prognosis” means the prediction/determination/assessment of the risks of progression of a disease in a subject. The prognosis comprises in particular the evaluation of the future development of the subject's condition and the possible chances of improvement or even cure. The prognosis can be determined on the basis of observations and/or measurements, carried out using different tools. In the case of neurodegenerative diseases, prognostic tools comprise tools that are also used for their diagnostic or stratification of subjects.

“Monitoring” means the determination/evaluation of the progression of a disease in a subject. Monitoring can be carried out on the basis of observations and/or measurements, carried out using different tools, at different time intervals. The intervals can be regular or irregular. Their frequency depends on the disease but also on the stage of progression of the disease. It can be of the order of a few days (for example in the event of a severe/advanced/serious stage of illness and/or in the event of a rapidly progressing illness and/or in the event of an exacerbation phase) to a few years (for example in cases of illness at an early, mild or moderate stage, and/or in cases of slowly progressive disease). In the case of neurodegenerative diseases, monitoring tools comprise tools that are also used for disease diagnostic or prognosis, or subject stratification.

“Evaluation of the efficacy of a treatment” means the determination of the clinical state of a subject subjected to treatment. Treatment can be preventive, for example in the case of a predisposition to a disease, or it can be curative, for example in the case of a diagnosed disease. The efficacy of the treatment can, for example, be assessed by determining the state of the subject at different time intervals. The condition of the subject can in particular be assessed before the first intake of the treatment then at regular (or irregular) time intervals after this first intake (for example after each new intake of the treatment). A comparison of the state of the subject assessed at these different intervals can then be carried out in order to identify a possible change. In the case of curative treatment, an improvement, absence of worsening, or worsening of the patient's condition less than that expected in the absence of treatment indicates that the treatment is effective, while a worsening of the patient's condition at least equal to that expected in the absence of treatment indicates that the treatment is not effective. In the case of preventive treatment, a lack of appearance of the disease or a later and/or less serious appearance than expected in the absence of treatment indicates that the treatment is effective, while an appearance which is as early and serious than expected in the absence of treatment indicates that the treatment is not effective. The patient's condition can be assessed on the basis of observations and/or measurements, carried out using different tools. In the case of neurodegenerative diseases, the tools for evaluating the efficacy of a treatment comprise tools that are also used for the diagnostic, prognosis or monitoring of the disease, or the stratification of subjects.

“Stage of a neurodegenerative disease” “stage of evolution of a neurodegenerative disease” or “stage of advancement of a neurodegenerative disease” means a phase of the neurodegenerative disease which is determined according to the severity of the symptoms from which suffers the subject and their implications/consequences on the subject's mode and/or quality of life. There can be four of these stages. For example:
At stage 1 (or first stage), this is called mild disease or mild stage.
At stage 2 (or second stage), this is called moderate disease or moderate stage.
At stage 3 (or third stage), this is called severe disease or severe stage.
At stage 4 (or fourth stage), this is called very severe disease or very severe stage. The quality of life is, at this stage, considerably impaired.

“Worsening” or “worsening phase”, means a period during which the clinical signs of a neurodegenerative disease increase in a subject suffering from said disease.

“Subject” or “patient” means a human individual or an animal other than a human. The subject is for example a human or an animal likely to be affected by a neurodegenerative disease or suffering from such a disease. The subject is preferably a human being. The subject can be a child (human subject aged 16 or younger) or an adult (human subject aged over 16). “Healthy subject” means a subject who does not suffer from the disease in question. In the context of the present invention, a healthy subject is preferably a subject who does not suffer from any neurodegenerative disease, more preferably a subject who does not suffer from any disease. “Reference subject” means a subject who suffers from a known neurodegenerative disease (in particular a synucleinopathy, and in particular Parkinson's disease (PD), dementia with Lewy bodies (DLB) or multiple system atrophy (MSA)), at a known stage.

“Biological sample” or “sample” from a subject means an entire organ or tissue or part of such an organ or tissue, a fluid or a fraction of such a fluid, cells or cellular components, obtained from this subject, as well as a homogenate, lysate or extract prepared therefrom. In particular, a “biological sample” or “sample” is preferably any tissue (preferably portions or fractions thereof) which may contain neurons and/or α-Syn proteins, including but in a non-limiting manner, a sample of the central nervous system (CNS), such as a brain sample or a sample of spinal cord, salivary glands, digestive system (for example colon), cerebrospinal fluid, plasma, blood, etc.

The biological sample may have been previously obtained by any technique known in the profession. These techniques comprise, for example, surgery (such as stereotactic surgery), puncture, explant, excision, biopsy. “Excision” means a surgical procedure consisting of cutting (excising) a more or less wide or deep part of the tissue, preferably an abnormality or growth of the tissue. An excision may be performed to remove and/or analyze a cancerous or suspicious tumor. The term “biopsy” here refers to a sample of cells or tissues taken for analysis. Several types of biopsy procedures are known and practiced in the field. The most common types comprise (1) incisional biopsy, in which only a sample of the tissue is taken; (2) excisional biopsy (or surgical biopsy), which consists of completely removing a tumor mass, thus performing a therapeutic and diagnostic procedure; and (3) needle biopsy, in which a tissue sample is taken using a needle, which can be large or fine. Other types of biopsy exist, such as smears or curettage, and can also be used to obtain the sample. Therefore, the sample can, for example, be an explant, an excision, a biopsy, etc. The sample is preferably obtained by a minimally invasive procedure, such as stereotaxic surgery.

“Identity” or “sequence identity” means an exact sequence match between two polypeptides or amino acids, or between two nucleic acid molecules or oligonucleotides. The identity percentages to which reference is made in the context of the presentation of the present invention are determined after optimal overall alignment of the sequences to be compared, which may therefore comprise one or more additions, deletions, truncations and/or substitutions. This identity percentage can be calculated by any sequence analysis method well known to the person skilled in the art. The identity percentage is determined after global alignment of the sequences to be compared taken in their entirety, over their entire length. Besides manually, it is possible to determine the overall sequence alignment using the Needleman and Wunsch (1970) algorithm.

In particular, for the nucleotide sequences, the comparison of the sequences can be carried out using any software well known to the person skilled in the art, such as for example the Needle software. The parameters used may in particular be the following: “Gap Open” equal to 10.0, “Gap Extend” equal to 0.5 and the EDNAFULL matrix (EMBOSS version of NCBI NUC4.4).

For amino acid sequences, the comparison of the sequences can be carried out using any software well known to the person skilled in the art, such as for example the Needle software. The parameters used may in particular be the following: “Gap Open” equal to 10.0, “Gap Extend” equal to 0.5 and the BLOSUM62 matrix.

As an illustration, “at least 80% sequence identity”, as used herein, represents in particular 80%, 81%, 82%, 83%, 84%, 85%, 86%, 87%, 88%, 89%, 90%, 91%, 92%, 93%, 94%, 95%, 96%, 97%, 98%, 99% or 100% sequence identity.

### Aptamers

In the context of the present invention, the inventors have developed modified ribonucleic acid (RNA) aptamers capable of specifically recognizing certain conformers of α-Syn fibers. The inventors have in fact selected and isolated modified RNA aptamers having different affinities for different α-Syn fiber conformers.

The inventors have in particular shown that, surprisingly, the aptamers developed are capable of distinguishing the conformers of F-type α-Syn fibers from conformers of R-type α-Syn fibers, unlike the DNA aptamers against α-Syn described in the prior art.

The data show in particular that the aptamers developed by the inventors have a strong affinity for the conformer of F-type α-Syn fibers (dissociation constant K_{d} between 5 and 10 nM). Unexpectedly, these aptamers, on the other hand, have very low or no affinity for the conformer of R-type α-Syn fibers, as well as the 65 and 91 α-syn fiber conformers. The inventors have also demonstrated that these aptamers recognize with an affinity at least 10 times lower the α-Syn protein in native form (monomeric, non-fibrillar form). Remarkably, the inventors have developed a method using a mixture of these aptamers which allows to effectively discriminate these fiber conformers by high-throughput sequencing, applicable to patient samples.

The data show that these aptamers are tools for specific and sensitive detection of different α-Syn fibers. The present invention therefore provides both effective and reliable diagnostic methods for neurodegenerative diseases, methods for screening molecules but also tools for research in the field of neurodegenerative diseases.

Aptamers have several advantages: 1) they have recognition affinities and specificity for their target comparable to those of antibodies; 2) being oligonucleotides, they can be used in numerous molecular biology techniques (quantitative PCR, chip, rolling circle amplification, high-throughput sequencing, etc.); 3) they are easy to synthesize or amplify in vitro; 4) they can be easily coupled to a large number of compounds; 5) they are poorly immunogenic; 6) they are not subject to denaturation problems during storage; 7) they are very resistant to temperature changes; 8) they are significantly cheaper than antibodies.

The present invention therefore relates to an aptamer characterized in that it has the ability to distinguish at least two fiber conformers of the α-Synuclein (α-Syn)protein, and in that it comprises, or essentially consists of, or consists of, a sequence specific for modified ribonucleic acid (RNA) having at least 85% identity with a sequence chosen from SEQ ID NO: 1 (specific sequence of aptamer N30), SEQ ID NO: 2 (specific sequence of aptamer N124), SEQ ID NO: 3 (specific sequence of aptamer N3), SEQ ID NO: 4 (specific sequence of aptamer 4F02), SEQ ID NO: 5 (specific sequence of aptamer 4F03), SEQ ID NO: 6 (specific sequence of aptamer F124), and SEQ ID NO: 7 (specific sequence of aptamer P65); preferably chosen from SEQ ID NO: 1, SEQ ID NO: 2, SEQ ID NO: 4, and SEQ ID NO: 5; more preferably chosen from SEQ ID NO: 1 and SEQ ID NO: 2.

In particular, the present invention relates to an aptamer characterized in that it has the ability to distinguish conformers of F-type α-Syn fibers of the α-Syn (α-Syn) protein from R-type α-Syn fibers, and in that it comprises, or essentially consists of, or consists of, a target/sequence specific for modified ribonucleic acid (RNA) having at least 85% identity with a sequence chosen from SEQ ID NO: 1 (specific sequence of aptamer N30), SEQ ID NO: 2 (specific sequence of aptamer N124), SEQ ID NO: 3 (specific sequence of aptamer N3), SEQ ID NO: 4 (specific sequence of aptamer 4F02), SEQ ID NO: 5 (specific sequence of aptamer 4F03), SEQ ID NO: 6 (specific sequence of aptamer F124), and SEQ ID NO: 7 (specific sequence of aptamer P65); preferably chosen from SEQ ID NO: 1, SEQ ID NO: 2, SEQ ID NO: 4 and SEQ ID NO: 5; more preferably chosen from SEQ ID NO: 1 and SEQ ID NO: 2.

According to a preferred embodiment, the aptamer comprises, or essentially consists of, or consists of, a sequence specific for modified RNA having at least 86% identity with a sequence chosen from SEQ ID NO: 1, SEQ ID NO: 2, SEQ ID NO: 3, SEQ ID NO: 4, SEQ ID NO: 5, SEQ ID NO: 6 and SEQ ID NO: 7; preferably chosen from SEQ ID NO: 1, SEQ ID NO: 2, SEQ ID NO: 4 and SEQ ID NO: 5; more preferably chosen from SEQ ID NO: 1 and SEQ ID NO: 2. Preferably, the aptamer comprises, or essentially consists of, or consists of, a sequence specific for modified RNA having at least 87% identity, more preferably at least 88% identity, more preferably at least 89% identity, more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, more preferably at least 99% identity, with a sequence specific for modified RNA chosen from SEQ ID NO: 1, SEQ ID NO: 2, SEQ ID NO: 3, SEQ ID NO: 4, SEQ ID NO: 5, SEQ ID NO: 6 and SEQ ID NO: 7; preferably chosen from SEQ ID NO: 1, SEQ ID NO: 2, SEQ ID NO: 4, and SEQ ID NO: 5; more preferably chosen from SEQ ID NO: 1, and SEQ ID NO: 2. Particularly preferably, the aptamer comprises, or essentially consists of, or consists of, a sequence specific for modified RNA chosen from SEQ ID NO: 1, SEQ ID NO: 2, SEQ ID NO: 3, SEQ ID NO: 4, SEQ ID NO: 5, SEQ ID NO: 6, and SEQ ID NO: 7; preferably chosen from SEQ ID NO: 1, SEQ ID NO: 2, SEQ ID NO: 4, and SEQ ID NO: 5; more preferably chosen from SEQ ID NO: 1, and SEQ ID NO: 2.

The sequences SEQ ID NO: 1, SEQ ID NO: 2, SEQ ID NO: 3, SEQ ID NO: 4, SEQ ID NO: 5, SEQ ID NO: 6 and SEQ ID NO: 7 are indicated in Table 1 below.

TABLE 1
Specific sequences of aptamers N30, N124, N3,
4F02, 4F03, F124 and P65.

| SEQ ID | Description | Sequence |
|---|---|---|
| SEQ ID | Specific sequence | UCGAUCCACGUCCGACAACGCGUU |
| NO: 1 | of aptamer N30 | UACUCGCCAUC |
| SEQ ID | Specific sequence | UCCGACCAUGCUUCAACUUAUACC |
| NO: 2 | of aptamer N124 | UCGGGGACUGU |
| SEQ ID | Specific sequence | CAACGCGUUUACCUCACACCACGU |
| NO: 3 | of aptamer N3 | CAUCCGUUGCC |
| SEQ ID | Specific sequence | GCUGGCAGCACGCACCGCUGACCGC |
| NO: 4 | of aptamer4F02 | UGGCUGCACUAUGCGUGUGGAGUGC |
| SEQ ID | Specific sequence | GCAACAGACGCACCGUACACACAUC |
| NO: 5 | of aptamer4F03 | UUGGCCGUUGGCUGCCCGACCAGCC |
| SEQ ID | Specific sequence | CACGGACACCUACCCGACGGCAUGU |
| NO: 6 | of aptamer F124 | CAGGACACAUGUUGUGCUCCGUGUG |
| SEQ ID | Specific sequence | CGCACAGUGUACACCUACACACAGC |
| NO: 7 | of aptamer P65 | AUACCUGUUGGCAUCCCAGGUUGCC |

According to one embodiment, the aptamer according to the invention also has the ability to distinguish conformers of F-type α-Syn fibers from α-Syn monomers. According to an alternative embodiment or in combination, the aptamer according to the invention also has the ability to distinguish the conformer of F-type α-Syn fibers from the 65 α-Syn fiber and/or 91 α-Syn fiber conformers. According to an alternative embodiment or in combination, the aptamer according to the invention also has the ability to distinguish conformers of F-type α-Syn fibers from α-Syn oligomers. Thus, according to one embodiment, the aptamer according to the invention has the ability to distinguish conformers of F-type α-Syn fibers from conformers of R-type α-Syn fibers, as well as the 65 α-Syn fiber conformers, 91 α-Syn fiber conformers, α-Syn oligomers and α-Syn monomers. Thus, according to an advantageous embodiment, the aptamer according to the invention is specific to the conformers of F-type α-Syn fibers. According to one embodiment, the aptamer according to the invention has no affinity for conformers of R-type α-Syn fibers, as well as for 65 α-Syn fiber conformers, 91 α-Syn fiber conformers, and α-Syn monomers.

According to a preferred embodiment, the dissociation constant K_{d(F)} of the aptamer according to the invention, measured for the conformers of F-type α-Syn fibers, is lower (preferably significantly lower) than the dissociation constant K_{d(R)} measured for the conformers of R-type α-Syn fibers, preferably lower by at least 10 times, more preferably lower by at least 11 times, more preferably lower by at least 12 times, more preferably lower by at least 13 times, more preferably lower by at least 14 times, more preferably lower by at least 15 times, more preferably lower by at least 20 times, more preferably lower by at least 25 times, more preferably lower by at least 30 times, more preferably lower by at least 40 times, more preferably lower by at least 50 times, more preferably lower by at least 100 times, more preferably lower by at least 200 times, more preferably lower by at least 300 times, more preferably lower by at least 400 times, more preferably lower by at least 500 times, more preferably lower by at least 600 times, more preferably lower by at least 700 times, more preferably lower by at least 800 times, more preferably lower by at least 900 times, more preferably lower by at least 1000 times. According to a particularly preferred embodiment, the affinity of the aptamer according to the invention for the conformers of R-type α-Syn fibers is so low that the dissociation constant K_{d(R)} cannot be measured/determined using the usual methods for determining dissociation constants (such as those listed in the definition section above).

According to an alternative preferred embodiment or in combination with the preferred embodiment above, the dissociation constant K_{d(F)} of the aptamer according to the invention, measured for the conformers of F-type α-Syn fibers, is lower (preferably significantly lower) than the dissociation constant K_{d(Mono)} measured for the α-Syn monomers, preferably lower by at least 2 times, preferably lower by at least 3 times, preferably lower by at least 4 times, preferably lower by at least 5 times, preferably lower by at least 6 times, preferably lower by at least 7 times, preferably lower by at least 8 times, preferably lower by at least 9 times, more preferably lower by at least 10 times, more preferably lower by at least 11 times, more preferably lower by at least 12 times, more preferably lower by at least 13 times, more preferably lower by at least 14 times, more preferably lower by at least 15 times, more preferably lower by at least 20 times, more preferably lower by at least 25 times, more preferably lower by at least 30 times, more preferably lower by at least 40 times, more preferably lower by at least 50 times, more preferably lower by at least 100 times, more preferably lower by at least 200 times, more preferably lower by at least 300 times, more preferably lower by at least 400 times, more preferably lower by at least 500 times, more preferably lower by at least 600 times, more preferably lower by at least 700 times, more preferably lower by at least 800 times, more preferably lower by at least 900 times, more preferably lower by at least 1000 times. According to a particularly preferred embodiment, the affinity of the aptamer according to the invention for α-Syn monomers is so low that the dissociation constant K_{d(mono)} cannot be measured/determined using the usual methods for determining dissociation constants (such as those listed in the definition section above).

According to an alternative preferred embodiment or in combination with one or more of the preferred embodiments above, the dissociation constant K_{d(F)} of the aptamer according to the invention, measured for the conformers of F-type α-Syn fibers, is less (preferably significantly less) than the dissociation constant K_{d(65)} measured for the 65 α-Syn fiber conformers, preferably lower by at least 2 times, preferably lower by at least 3 times, preferably lower by at least 4 times, preferably lower by at least 5 times, preferably lower by at least 6 times, preferably lower by at least 7 times, preferably lower by at least 8 times, preferably lower by at least 9 times, more preferably lower by at least 10 times, more preferably lower by at least 11 times, more preferably lower by at least 12 times, preferably even lower by at least 13 times, more preferably lower by at least 14 times, more preferably lower by at least 15 times, more preferably lower by at least 20 times, more preferably lower by at least 25 times, more preferably lower by at least 30 times, more preferably lower by at least 40 times, more preferably lower by at least 50 times, more preferably lower by at least 100 times, more preferably lower by at least 200 times, more preferably lower by at least 300 times, more preferably lower by at least 400 times, more preferably lower by at least 500 times, more preferably lower by at least 600 times, more preferably lower by at least 700 times, more preferably lower by at least 800 times, more preferably lower by at least 900 times, more preferably lower by at least 1000 times. According to a particularly preferred embodiment, the affinity of the aptamer according to the invention for the 65 α-Syn fiber conformers is so low that the dissociation constant K_{d(65)} cannot be measured/determined using the usual methods for determining dissociation constants (such as those listed in the definition section above).

According to an alternative preferred embodiment or in combination with one or more of the embodiments above, the dissociation constant K_{d(F)} of the aptamer according to the invention, measured for the conformers of F-type α-Syn fibers, is lower (preferably significantly lower) than the dissociation constant K_{d(91)} measured for the 91 α-Syn fiber conformers of, preferably lower by at least 2 times, preferably lower by at least 3 times, preferably lower by at least 4 times, preferably lower by at least 5 times, preferably lower by at least 6 times, preferably lower by at least 7 times, preferably lower by at least 8 times, preferably lower by at least 9 times, more preferably lower by at least 10 times, more preferably lower by at least 11 times, more preferably lower by at least 12 times, more preferably lower by at least 13 times, more preferably lower by at least 14 times, more preferably lower by at least 15 times, more preferably lower by at least 20 times, more preferably lower by at least 25 times, more preferably lower by at least 30 times, more preferably lower by at least 40 times, more preferably lower by at least 50 times, more preferably lower by at least 100 times, more preferably lower by at least 200 times, more preferably lower by at least 300 times, more preferably lower by at least 400 times, more preferably lower by at least 500 times, more preferably lower by at least 600 times, more preferably lower by at least 700 times, more preferably lower by at least 800 times, more preferably lower by at least 900 times, more preferably lower by at least 1000 times. According to a particularly preferred embodiment, the affinity of the aptamer according to the invention for the 91 α-Syn fiber conformers is so low that the dissociation constant K_{d(91)} cannot be measured/determined using the usual methods for determining dissociation constants (such as those listed in the definition section above).

According to an alternative preferred embodiment or in combination with one or more of the embodiments above, the dissociation constant K_{d(F)} of the aptamer according to the invention, measured for the conformers of F-type α-Syn fibers, is lower (preferably significantly lower) than the dissociation constant K_{d(Random)} of a random aptamer (that is to say an aptamer comprising a random sequence of modified RNA, preferably as defined below in the section “composition and kits”) measured for conformers of F-type α-Syn fibers, preferably lower by at least 2 times, preferably lower by at least 3 times, preferably lower by at least 4 times, preferably lower by at least 5 times, preferably lower by at least 6 times, preferably lower by at least 7 times, preferably lower by at least 8 times, preferably lower by at least 9 times, more preferably lower by at least 10 times, more preferably lower by at least 11 times, more preferably lower by at least 12 times, more preferably lower by at least 13 times, more preferably lower by at least 14 times, more preferably lower by at least 15 times, more preferably lower by at least 20 times, more preferably lower by at least 25 times, more preferably lower by at least 30 times, more preferably lower by at least 40 times, more preferably lower by at least 50 times, more preferably lower by at least 100 times, more preferably lower by at least 200 times, more preferably lower by at least 300 times, more preferably lower by at least 400 times, more preferably lower by at least 500 times, more preferably lower by at least 600 times, more preferably lower by at least 700 times, more preferably lower by at least 800 times, more preferably lower by at least 900 times, more preferably lower by at least 1000 times. According to a particularly preferred embodiment, the affinity of the random aptamer for the conformers of F-type α-Syn fibers is so low that the dissociation constant K_{d(random)} cannot be measured/determined using the usual methods for determining dissociation constants (such as those listed in the definition section above).

Advantageously, the dissociation constant K_{d(F)} measured, in particular using the usual methods for determining dissociation constants (such as those listed in the definition section above), for the conformers of F-type α-Syn fibers, is less than 50 nM, more preferably less than 40 nM, more preferably less than 30 nM, more preferably less than 25 nM, more preferably less than 20 nM, more preferably less than 18 nM, of more preferably less than 16 nM, more preferably less than 14 nM, more preferably less than 13 nM, more preferably less than 12 nM, more preferably less than 11 nM, more preferably less than 10 nM, more preferably less than 9 nM, more preferably less than 8 nM, more preferably less than 7 nM. Advantageously, the dissociation constant K_{d(R)} measured, in particular using the usual methods for determining dissociation constants (such as those listed in the definition section above), for the conformers of R-type α-Syn fibers, is greater than 100 nM, more preferably greater than 150 nM, more preferably greater than 200 nM, more preferably greater than 300 nM, more preferably greater than 400 nM, more preferably greater than 500 nM, more preferably greater than 600 nM, more preferably greater than 700 nM, more preferably greater than 800 nM, more preferably greater than 900 nM, more preferably greater than 1000 nM. According to a particularly preferred embodiment, the affinity of the aptamer according to the invention for the conformers of R-type α-Syn fibers is so low that the dissociation constant K_{d(R)} cannot be measured/determined using the usual methods for determining dissociation constants (such as those listed in the definition section above).

According to a preferred embodiment, the aptamer according to the invention has at least one dissociation constant K_{d} as follows:
a) the dissociation constant K_{d(F)} measured for the conformers of F-type α-Syn fibers is less than 15 nM, preferably less than 10 nM; and/or
b) the dissociation constant K_{d(R)} measured for the conformers of R-type α-Syn fibers is greater than 100 nM, preferably greater than 500 nM.

The dissociation constant K_{d} of an aptamer is for example measured using the usual methods for determining dissociation constants, preferably chosen from those listed in the definition section above, more preferably by filtration, more preferably by filtration on nitrocellulose membrane.

According to a preferred embodiment, the aptamer according to the invention further comprises:
i. in 5′ of the specific sequence, a primer sequence 5′ of modified RNA having at least 85% identity, preferably at least 86% identity, preferably at least 87% identity, preferably at least 88% identity, preferably at least 89% identity, preferably at least 90% identity, preferably at least 91% identity, preferably at least 92% identity, preferably at least 93% identity, preferably at least 94% identity, preferably at least 95% identity, preferably at least 96% identity, preferably at least 97% identity, preferably at least 98% identity, preferably at least 99% identity, preferably 100% identity, with a sequence chosen from SEQ ID NO: 29 (primer sequence P72 of aptamers N, in 5′), and SEQ ID NO: 30 (primer sequence P73 of aptamers 4F, F, R and P, in 5′), preferably located at the end 5′ of the specific sequence; and/or
ii. in 3′ of the specific sequence, a primer sequence 3′ of modified RNA having at least 85% identity, preferably at least 86% identity, preferably at least 87% identity, preferably at least 88% identity, preferably at least 89% identity, preferably at least 90% identity, preferably at least 91% identity, preferably at least 92% identity, preferably at least 93% identity, preferably at least 94% identity, preferably at least 95% identity, preferably at least 96% identity, preferably at least 97% identity, preferably at least 98% % identity, preferably at least 99% identity, preferably 100% identity, with a sequence chosen from SEQ ID NO: 31 (primer PiRT in 3′), SEQ ID NO: 32 (Sequence G of 24 nts in 3′), and SEQ ID NO: 33 (PiRT-G; primer PiTR+sequence G combination, in 3′), preferably located at the end 3′ of the specific sequence.

According to embodiment i. above, the aptamer is in the form “Primer 5′-Specific sequence” (in the 5′-3′ direction of the modified RNA sequence). According to embodiment ii. above in relation to the primer of SEQ ID NO: 31, the aptamer is in the form “Specific sequence-Primer 3′” (in the 5′-3′ direction of the modified RNA sequence). According to embodiment i. combined with embodiment ii. above in relation to the primer of SEQ ID NO: 31, the aptamer is in the form “Primer 5′-Specific sequence-Primer 3′” (in the 5′-3′ direction of the sequence of modified RNA), the latter form being hereinafter called “complete sequence of the aptamer” (or “combined sequence of the aptamer”).

The sequences SEQ ID NO: 29 to SEQ ID NO: 33 are indicated in Table 2 below.

TABLE 2
Sequences of the primers 3′ and 5′ of
modified RNA aptamers.

| SEQ ID | Description | Sequence |
|---|---|---|
| SEQ ID | Primer sequence P72 of | GGGAGAGUAUCCGUU |
| NO: 29 | aptamers N, in 5′ | GAGGCUGA |
| SEQ ID | Primer sequence P73 of | GGGAGAGUAUCCGUU |
| NO: 30 | aptamers 4F, F, R and P, | GGAGGCAU |
| | in 5′ | |
| SEQ ID | Primer sequence PiRT in 3′ | AGAUCGGAAGAGCGU |
| NO: 31 | | CGUGUAGG |
| SEQ ID | Sequence G of 24 nts in 3′ | GCAUUGCCCCAGCGU |
| NO: 32 | | GACUGCCUA |
| SEQ ID | Sequence PiRT-G: primer | AGAUCGGAAGAGCGU |
| NO: 33 | PITR + sequence G | CGUGUAGGGCAUUGC |
| | combination, in 3′ | CCCAGCGUGACUGCC |
| | | UA |

According to a particularly advantageous embodiment, the aptamer according to the invention comprises, or essentially consists of, or consists of, a complete sequence of modified RNA having at least 85% identity with a sequence chosen from SEQ ID NO: 34 (complete sequence of aptamer N30, with primers 5′ and 3′, but without sequence G), SEQ ID NO: 35 (complete sequence of aptamer N124), SEQ ID NO: 36 (complete sequence of aptamer N3), SEQ ID NO: 37 (complete sequence of aptamer 4F02), SEQ ID NO: 38 (complete sequence of aptamer 4F03), SEQ ID NO: 39 (complete sequence of aptamer F124), and SEQ ID NO: 40 (complete sequence of the aptamer P65), preferably chosen from SEQ ID NO: 34, SEQ ID NO: 35, SEQ ID NO: 37, and SEQ ID NO: 38; more preferably chosen from SEQ ID NO: 34 and SEQ ID NO: 35.

According to a preferred embodiment, the aptamer comprises, or essentially consists of, or consists of, a complete sequence of modified RNA having at least 86% identity with a sequence chosen from SEQ ID NO: 34, SEQ ID NO: 35, SEQ ID NO: 36, SEQ ID NO: 37, SEQ ID NO: 38, SEQ ID NO: 39, and SEQ ID NO: 40; preferably chosen from SEQ ID NO: 34, SEQ ID NO: 35, SEQ ID NO: 37, and SEQ ID NO: 38; more preferably chosen from SEQ ID NO: 34 and SEQ ID NO: 35. Preferably the aptamer comprises, or essentially consists of, or consists of, a complete sequence of modified RNA having at least 87% identity, more preferably at least 88% identity, more preferably at least 89% identity, more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, more preferably at least 99% identity, with a complete modified RNA sequence chosen from SEQ ID NO: 34, SEQ ID NO: 35, SEQ ID NO: 36, SEQ ID NO: 37, SEQ ID NO: 38, SEQ ID NO: 39, and SEQ ID NO: 40; preferably chosen from SEQ ID NO: 34, SEQ ID NO: 35, SEQ ID NO: 37, and SEQ ID NO: 38; more preferably chosen from SEQ ID NO: 34 and SEQ ID NO: 35. Particularly preferably, the aptamer comprises, or essentially consists of, or consists of, a complete sequence of modified RNA chosen from SEQ ID NO: 34, SEQ ID NO: 35, SEQ ID NO: 36, SEQ ID NO: 37, SEQ ID NO: 38, SEQ ID NO: 39, and SEQ ID NO: 40; preferably chosen from SEQ ID NO: 34, SEQ ID NO: 35, SEQ ID NO: 37, and SEQ ID NO: 38; more preferably chosen from SEQ ID NO: 34 and SEQ ID NO: 35.

The sequences SEQ ID NO: 34, SEQ ID NO: 35, SEQ ID NO: 36, SEQ ID NO: 37, SEQ ID NO: 38, SEQ ID NO: 39, and SEQ ID NO: 40 are indicated in Table 3 below.

TABLE 3
Complete sequence of aptamers N30, N124, N3,
4F02, 4F03, F124 and P65.

| SEQ ID | Description | Sequence |
|---|---|---|
| SEQ ID | Complete | GGGAGAGUAUCCGUUGAGGCUGAUCGAUCC |
| NO: 34 | sequence of | ACGUCCGACAACGCGUUUACUCGCCAUCAG |
| | aptamer | AUCGGAAGAGCGUCGUGUAGG |
| | N30 | |
| SEQ ID | Complete | GGGAGAGUAUCCGUUGAGGCUGAUCCGACC |
| NO: 35 | sequence of | AUGCUUCAACUUAUACCUCGGGGACUGUAG |
| | aptamer | AUCGGAAGAGCGUCGUGUAGG |
| | N124 | |
| SEQ ID | Complete | GGGAGAGUAUCCGUUGAGGCUGACAACGCG |
| NO: 36 | sequence of | UUUACCUCACACCACGUCAUCCGUUGCCAG |
| | aptamer N3 | AUCGGAAGAGCGUCGUGUAGG |
| SEQ ID | Complete | GGGAGAGUAUCCGUUGGAGGCAUGCUGGCA |
| NO: 37 | sequence of | GCACGCACCGCUGACCGCUGGCUGCACUAU |
| | aptamer | GCGUGUGGAGUGCAGAUCGGAAGAGCGUCG |
| | 4F02 | UGUAGG |
| SEQ ID | Complete | GGGAGAGUAUCCGUUGGAGGCAUGCAACAG |
| NO: 38 | sequence of | ACGCACCGUACACACAUCUUGGCCGUUGGC |
| | aptamer | UGCCCGACCAGCCAGAUCGGAAGAGCGUCG |
| | 4F03 | UGUAGG |
| SEQ ID | Complete | GGGAGAGUAUCCGUUGGAGGCAUCACGGAC |
| NO: 39 | sequence of | ACCUACCCGACGGCAUGUCAGGACACAUGU |
| | aptamer | UGUGCUCCGUGUGAGAUCGGAAGAGCGUCG |
| | F124 | UGUAGG |
| SEQ ID | Complete | GGGAGAGUAUCCGUUGGAGGCAUCGCACAG |
| NO: 40 | sequence of | UGUACACCUACACACAGCAUACCUGUUGGC |
| | aptamer | AUCCCAGGUUGCCAGAUCGGAAGAGCGUCG |
| | P65 | UGUAGG |

The aptamer according to the invention may further comprise, in 3′ of the complete sequence “Primer 5′-Specific Sequence-Primer 3′” as defined above, a primer sequence 3′ of modified RNA having at least 85% identity, preferably at least 86% identity, preferably at least 87% identity, preferably at least 88% identity, preferably at least 89% identity, preferably at least 90% identity, preferably at least 91% identity, preferably at least 92% identity, preferably at least 93% identity, preferably at least 94% identity, preferably at least 95% identity, preferably at least 96% identity, preferably at least 97% identity, preferably at least 98% identity, preferably at least 99% identity, preferably 100% identity, with SEQ ID NO: 32, preferably located at the end 3′ of the aptamer. According to this embodiment, the aptamer is in the form “Primer 5′-Specific Sequence-Primer 3′-Sequence G” (in the 5′-3′ direction of the modified RNA sequence), this form being hereinafter called “complete aptamer sequence G” (or combined aptamer sequence G).

According to a preferred embodiment, the RNA of the aptamer according to the invention (that is to say of any aptamer as described above, including the specific sequence of the aptamer, the sequence of the primers 5′ and/or 3′, the complete sequence of the aptamer, and the complete sequence G of the aptamer) was modified in order to increase its resistance to RNA nucleases. Advantageously, the RNA of the aptamer according to the invention was modified by at least one modification chosen from: modification of the OH function on the carbon in the position 2′ of the ribose by methylation; the substitution of the OH function on the carbon in the position 2′ of the ribose by an O-Methoxyethyl group; the substitution of the OH function on the carbon in the position 2′ of the ribose by an amino group; the substitution of the OH function on the carbon in the position 2′ of the ribose by a halogen (in particular by fluorine); the replacement of the phosphodiester (PO) by a phosphorothioate (PS) group (therefore this is referred to as a phosphorothioate skeleton); the use of a Locked Nucleic Acid (LNA) type structure, that is to say the formation of a methylene bridge in order to lock the ribose in the C3′-endo (N-type) conformation; the use of a Peptide Nucleic Acid (PNA) type structure, that is to say the replacement of the sugar-phosphate backbone with a peptide type backbone; and any combination thereof. The RNA of the aptamer according to the invention was preferably modified by at least one modification chosen from modification of the OH function on the carbon in the position 2′ of the ribose by methylation; the substitution of the OH function on the carbon in the position 2′ of the ribose by an O-Methoxyethyl group; the substitution of the OH function on the carbon in the position 2′ of the ribose by an amino group; the substitution of the OH function on the carbon in the position 2′ of the ribose by a halogen (in particular by fluorine); and any combination thereof. According to a particularly preferred embodiment of the aptamer according to the invention, the riboses of the pyrimidines carry a fluorine atom on the carbon in the position 2′.

Thus, preferably, the modified RNA of the aptamer according to the invention is an RNA the riboses of pyrimidine nucleotides of which carry a fluorine atom on the carbon in position 2′, preferably in which the riboses of the purine nucleotides are unchanged (it is therefore a 2′Fluoro-pyrimidine RNA (2′F-PyRNA)).

According to one embodiment, the aptamer according to the invention further comprises at least one additional group as defined above in the “Definitions” section. The additional group can be added to any nucleotide of the aptamer. The additional group(s) is(are) preferably located at the end 3′ of the aptamer, or at the end 5′ of the aptamer, or at the end 3′ and at the end 5′ of the aptamer.

### Compositions and Kits

The inventors have developed aptamers having a strong affinity for the conformer of F-type α-Syn fibers (dissociation constant K_{d} between 5 and 10 nM). Unexpectedly, these aptamers, on the other hand, have very low or no affinity for the conformers of R-type α-Syn fibers, as well as 65 and 91 α-syn fibers. The inventors have also demonstrated that these aptamers recognize with an affinity at least 10 times lower the α-Syn protein in native form (monomeric, non-fibrillar form). Remarkably, the inventors have developed a method using a mixture of these aptamers which allows to effectively discriminate these fiber conformers by high-throughput sequencing, applicable to patient samples.

The present invention therefore relates to a composition comprising, or essentially consisting of, at least one aptamer according to the invention (as defined above).

The present invention further relates to a kit comprising, or essentially consisting of, at least one aptamer according to the invention (as defined above).

According to one embodiment, the composition or the kit further comprises at least one additional aptamer chosen from aptamers comprising, or essentially consisting of, or consisting of, a sequence specific for modified RNA having at least 85% identity with a sequence chosen from SEQ ID NO: 8 (specific sequence of aptamer NO), SEQ ID NO: 9 (specific sequence of aptamer N1), SEQ ID NO: 10 (specific sequence of aptamer N2), SEQ ID NO: 11 (specific sequence of aptamer N4), SEQ ID NO: 12 (specific sequence of aptamer N5), SEQ ID NO: 13 (specific sequence of aptamer N15), SEQ ID NO: 14 (specific sequence of aptamer N20), SEQ ID NO: 15 (specific sequence of aptamer N37), SEQ ID NO: 16 (specific sequence of aptamer N62) and SEQ ID NO: 17 (specific sequence of aptamer N73), SEQ ID NO: 18 (specific sequence of aptamer N164), SEQ ID NO: 19 (specific sequence of aptamer 4F01), SEQ ID NO: 20 (specific sequence of aptamer 4F04), SEQ ID NO: 21 (specific sequence of aptamer 4F05), SEQ ID NO: 22 (specific sequence of aptamer R01), SEQ ID NO: 23 (specific sequence of aptamer R02), SEQ ID NO: 24 (specific sequence of aptamer R03), SEQ ID NO: 25 (specific sequence of aptamer R04), SEQ ID NO: 26 (specific sequence of aptamer R05), SEQ ID NO: 27 (specific sequence of aptamer R84), and SEQ ID NO: 28 (specific sequence of aptamer P91);
the composition or kit preferably further comprising at least one aptamer comprising a modified RNA random sequence.

According to a preferred embodiment, the additional aptamer comprises, or essentially consists of, or consists of, a sequence specific for modified RNA having at least 86% identity with a sequence chosen from SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28. Preferably the additional aptamer comprises, or essentially consists of, or consists of, a sequence specific for modified RNA having at least 87% identity, more preferably at least 88% identity, more preferably at least 89% identity, more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, more preferably at least 99% identity, with a sequence chosen from SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28. Particularly preferably, the additional aptamer comprises, or essentially consists of, or consists of, a sequence specific for modified RNA chosen from SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28.

The sequences SEQ ID NO: 8 to 28 are indicated in Table 4 below.

TABLE 4
Specific sequences of additional aptamers N0, N1,
N2, N4, N5, N15, N20, N37, N62 N73, N164, 4F01,
4F04, 4F05, R01, R02, R03, R04, R05, R84 and P91

| SEQ ID | Description | Sequence |
|---|---|---|
| SEQ ID | Specific | UCAGUCCAGUACGAGACGCGUUUACCUCC |
| NO: 8 | sequence of | ACUGCA |
| | aptamer N0 | |
| SEQ ID | Specific | AUCCGACCAACCCAACGCGUUUACCUCAC |
| NO: 9 | sequence of | CUGCA |
| | aptamer N1 | |
| SEQ ID | Specific | UUCCGACCGCCGCAACUUAUAGGUAUCCC |
| NO: 10 | sequence of | GCUGCA |
| | aptamer N2 | |
| SEQ ID | Specific | ACGCGUUUACUCCGCUAGUACGAACCCGA |
| NO: 11 | sequence of | UUGCCC |
| | aptamer N4 | |
| SEQ ID | Specific | AGCCAUGCUGUCAACUUUUAACUCGUCAC |
| NO: 12 | sequence of | CGCUCG |
| | aptamer N5 | |
| SEQ ID | Specific | AUCGGCCACAGUCGACAACUUUGAAAUCC |
| NO: 13 | sequence of | ACCUGC |
| | aptamer N15 | |
| SEQ ID | Specific | GCAGCACAUGACCUCACCUUUUACUCUGC |
| NO: 14 | sequence of | GCUGCA |
| | aptamer N20 | |
| SEQ ID | Specific | UCAGUCCAGCACCAACGCCGUUUGCUCUC |
| NO: 15 | sequence of | GACUAC |
| | aptamer N37 | |
| SEQ ID | Specific | GUUUCCGAACGGCCCAACUUUGAAAUCCC |
| NO: 16 | sequence of | CGCCCG |
| | aptamer N62 | |
| SEQ ID | Specific | CAACUUGAAAUCCCAACCCUGCAGCCGUG |
| NO: 17 | sequence of | UCUGGU |
| | aptamer N73 | |
| SEQ ID | Specific | GUUUCCGACCACGACCCAACGUUACUGCC |
| NO: 18 | sequence of | CACCAC |
| | aptamer N164 | |
| SEQ ID | Specific | AGCAGCACACGACCAGUGUGCCCCACACC |
| NO: 19 | sequence of | CAGUGGUGGUCUGUGGUGUGC |
| | aptamer 4F01 | |
| SEQ ID | Specific | CCGUCCACCAGACCAACGUACAAACUCCG |
| NO: 20 | sequence of | CUGGUGGUCGCCUACCCUGGC |
| | aptamer 4F04 | |
| SEQ ID | Specific | GCCGCAGGCUACACCACAGCUUCCCCUUC |
| NO: 21 | sequence of | AGCGUGUUGUGGAUAUCCGGC |
| | aptamer 4F05 | |
| SEQ ID | Specific | UGCCGCACUACAGCUUGGUCUGCAAUUCC |
| NO: 22 | sequence of | UCUGCGCACAGCUCCAUGUGC |
| | aptamer R01 | |
| SEQ ID | Specific | GCACGAUGUCCAUGACCAACUCCAGUCAC |
| NO: 23 | sequence of | GGCCCUGCAGCGUUAGGCUGU |
| | aptamer R02 | |
| SEQ ID | Specific | CCAGCAUCACCAGCGGCACGACGUCGGAC |
| NO: 24 | sequence of | GGCUGGCUGGUCCGUCACCGU |
| | aptamer R03 | |
| SEQ ID | Specific | GCAGAGCUACACGGUGCAAGUAGCACGUC |
| NO: 25 | sequence of | CUGCCAUGCAUGCAGUGCUGC |
| | aptamer R04 | |
| SEQ ID | Specific | CGGGAAGCAGCACGACGGCCUCAAUGCAC |
| NO: 26 | sequence of | UUGCCGGUUGGUUUCGGCUGC |
| | aptamer R05 | |
| SEQ ID | Specific | CUGCGAAGUGCCCAAGACCAUAUCCACUG |
| NO: 27 | sequence of | CACACGACAGCUGAUGGUGGC |
| | aptamer R84 | |
| SEQ ID | Specific | GCUACACACAUCGCACGUCACCCACUAUG |
| NO: 28 | sequence of | GGGACAUCUUGCGGCGUGC |
| | aptamer P91 | |

According to one embodiment, at least one additional aptamer of the composition or of the kit according to the invention further comprises:
i. in 5′ of the specific sequence, a primer sequence 5′ of modified RNA having at least 85% identity, preferably at least 86% identity, preferably at least 87% identity, preferably at least 88% identity, preferably at least 89% identity, preferably at least 90% identity, preferably at least 91% identity, preferably at least 92% identity, preferably at least 93% identity, preferably at least 94% identity, preferably at least 95% identity, preferably at least 96% identity, preferably at least 97% identity, preferably at least 98% identity, preferably at least 99% identity, preferably 100% identity, with a sequence chosen from SEQ ID NO: 29 (primer sequence P72 of aptamers N, in 5′) and SEQ ID NO: 30 (primer sequence P73 of aptamers 4F, F, R and P, in 5′), preferably located at the end 5′ of the specific sequence; and/or
ii. in 3′ of the specific sequence, a primer sequence 3′ of modified RNA having at least 85% identity, preferably at least 86% identity, preferably at least 87% identity, preferably at least 88% identity, preferably at least 89% identity, preferably at least 90% identity, preferably at least 91% identity, preferably at least 92% identity, preferably at least 93% identity, preferably at least 94% identity, preferably at least 95% identity, preferably at least 96% identity, preferably at least 97% identity, preferably at least 98% % identity, preferably at least 99% identity, preferably 100% identity, with a sequence chosen from SEQ ID NO: 31 (primer PiRT in 3′), SEQ ID NO: 32 (Sequence G of 24 nts in 3′) and SEQ ID NO: 33 (PiRT-G; primer PiTR+sequence G combination, in 3′), preferably located at the end 3′ of the specific sequence.

According to embodiment i. above, the additional aptamer is in the form “Primer 5′-Additional aptamer specific sequence” (in the 5′-3′ direction of the modified RNA sequence). According to embodiment ii. above in relation to the primer of SEQ ID NO: 31, the additional aptamer is in the form “Additional aptamer specific sequence-primer 3′” (in the 5′-3′ direction of the modified RNA sequence). According to embodiment i. combined with embodiment ii. above in relation to the primer of SEQ ID NO: 31, the additional aptamer is in the form “Primer 5′-Additional aptamer specific sequence-Primer 3′” (in the 5′-3′ direction of the modified RNA sequence), the latter form being hereinafter called “complete sequence of the additional aptamer” (or combined sequence of the additional aptamer). The sequences SEQ ID NO: 29 to SEQ ID NO: 33 are indicated in Table 2 above.

According to a particularly advantageous embodiment, at least one additional aptamer of the composition or of the kit according to the invention is chosen from aptamers comprising, or essentially consisting of, or consisting of, a complete modified RNA sequence having at least 85% identity with a sequence chosen from SEQ ID NO: 41 (complete sequence of aptamer NO, with primers 5′ and 3′, but without sequence G), SEQ ID NO: 42 (complete sequence of aptamer N1), SEQ ID NO: 43 (complete sequence of aptamer N2), SEQ ID NO: 44 (complete sequence of aptamer N4), SEQ ID NO: 45 (complete sequence of aptamer N5), SEQ ID NO: 46 (complete sequence of aptamer N15), SEQ ID NO: 47 (complete sequence of aptamer N20), SEQ ID NO: 48 (complete sequence of aptamer N37), SEQ ID NO: 49 (complete sequence of aptamer N62) and SEQ ID NO: 50 (complete sequence of aptamer N73), SEQ ID NO: 51 (complete sequence of aptamer N164), SEQ ID NO: 52 (complete sequence of aptamer 4F01), SEQ ID NO: 53 (complete sequence of aptamer 4F04), SEQ ID NO: 54 (complete sequence of aptamer 4F05), SEQ ID NO: 55 (complete sequence of aptamer R01), SEQ ID NO: 56 (complete sequence of aptamer R02), SEQ ID NO: 57 (complete sequence of aptamer R03), SEQ ID NO: 58 (complete sequence of aptamer R04), SEQ ID NO: 59 (complete sequence of aptamer R05), SEQ ID NO: 60 (complete sequence of aptamer R84), and SEQ ID NO: 61 (complete sequence of aptamer P91).

According to a preferred embodiment, at least one additional aptamer comprises, or essentially consists of, or consists of, a complete sequence of modified RNA having at least 86% identity with a sequence chosen from SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61. Preferably the additional aptamer comprises, or essentially consists of, or consists of, a complete modified RNA sequence having at least 87% identity, more preferably at least 88% identity, more preferably at least 89% identity, more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, more preferably at least 99% identity, with a complete modified RNA sequence chosen from SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61. Particularly preferably, the at least additional aptamer comprises, or essentially consists of, or consists of, a complete modified RNA sequence chosen from SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61.

The sequences SEQ ID NO: 41 to 61 are indicated in Table 5 below.

TABLE 5
Complete sequence of additional aptamers NO, N1,
N2, N4, N5, N15, N20, N37, N62 N73, N164, 4F01,
4F04, 4F05, R01, R02, R03, R04, R05, R84 and P91

| SEQ ID | Description | Sequence |
|---|---|---|
| SEQ ID | Complete | GGGAGAGUAUCCGUUGAGGCUGAUCAGUCCAGUACGAGACGCGU |
| NO: 41 | sequence of | UUACCUCCACUGCAAGAUCGGAAGAGCGUCGUGUAGG |
| | aptamer NO | |
| SEQ ID | Complete | GGGAGAGUAUCCGUUGAGGCUGAAUCCGACCAACCCAACGCGUU |
| NO: 42 | sequence of | UACCUCACCUGCAAGAUCGGAAGAGCGUCGUGUAGG |
| | aptamer N1 | |
| SEQ ID | Complete | GGGAGAGUAUCCGUUGAGGCUGAUUCCGACCGCCGCAACUUAUA |
| NO: 43 | sequence of | GGUAUCCCGCUGCAAGAUCGGAAGAGCGUCGUGUAGG |
| | aptamer N2 | |
| SEQ ID | Complete | GGGAGAGUAUCCGUUGAGGCUGAACGCGUUUACUCCGCUAGUAC |
| NO: 44 | sequence of | GAACCCGAUUGCCCAGAUCGGAAGAGCGUCGUGUAGG |
| | aptamer N4 | |
| SEQ ID | Complete | GGGAGAGUAUCCGUUGAGGCUGAAGCCAUGCUGUCAACUUUUAA |
| NO: 45 | sequence of | CUCGUCACCGCUCGAGAUCGGAAGAGCGUCGUGUAGG |
| | aptamer N5 | |
| SEQ ID | Complete | GGGAGAGUAUCCGUUGAGGCUGAAUCGGCCACAGUCGACAACUU |
| NO: 46 | sequence of | UGAAAUCCACCUGCAGAUCGGAAGAGCGUCGUGUAGG |
| | aptamer N15 | |
| SEQ ID | Complete | GGGAGAGUAUCCGUUGAGGCUGAGCAGCACAUGACCUCACCUUU |
| NO: 47 | sequence of | UACUCUGCGCUGCAAGAUCGGAAGAGCGUCGUGUAGG |
| | aptamer N20 | |
| SEQ ID | Complete | GGGAGAGUAUCCGUUGAGGCUGAUCAGUCCAGCACCAACGCCGU |
| NO: 48 | sequence of | UUGCUCUCGACUACAGAUCGGAAGAGCGUCGUGUAGG |
| | aptamer N37 | |
| SEQ ID | Complete | GGGAGAGUAUCCGUUGAGGCUGAGUUUCCGAACGGCCCAACUUU |
| NO: 49 | sequence of | GAAAUCCCCGCCCGAGAUCGGAAGAGCGUCGUGUAGG |
| | aptamer N62 | |
| SEQ ID | Complete | GGGAGAGUAUCCGUUGAGGCUGACAACUUGAAAUCCCAACCCUG |
| NO: 50 | sequence of | CAGCCGUGUCUGGUAGAUCGGAAGAGCGUCGUGUAGG |
| | aptamer N73 | |
| SEQ ID | Complete | GGGAGAGUAUCCGUUGAGGCUGAGUUUCCGACCACGACCCAACG |
| NO: 51 | sequence of | UUACUGCCCACCACAGAUCGGAAGAGCGUCGUGUAGG |
| | aptamer N164 | |
| SEQ ID | Complete | GGGAGAGUAUCCGUUGGAGGCAAUAGCAGCACACGACCAGUGUG |
| NO: 52 | sequence of | CCCCACACCCAGUGGUGGUCUGUGGUGUGCAGAUCGGAAGAGCG |
| | aptamer 4F01 | UCGUGUAGG |
| SEQ ID | Complete | GGGAGAGUAUCCGUUGGAGGCAUCCGUCCACCAGACCAACGUAC |
| NO: 53 | sequence of | AAACUCCGCUGGUGGUCGCCUACCCUGGCAGAUCGGAAGAGCGU |
| | aptamer 4F04 | CGUGUAGG |
| SEQ ID | Complete | GGGAGAGUAUCCGUUGGAGGCAUGCCGCAGGCUACACCACAGCU |
| NO: 54 | sequence of | UCCCCUUCAGCGUGUUGUGGAUACUCGGCAGAUCGGAAGAGCGU |
| | aptamer 4F05 | CGUGUAGG |
| SEQ ID | Complete | GGGAGAGUAUCCGUUGGAGGCAUUGCCGCACUACAGCUUGGUCU |
| NO: 55 | sequence of | GCAAUUCCUCUGCGCACAGCUCCAUGUGCAGAUCGGAAGAGCGU |
| | aptamer R01 | CGUGUAGG |
| SEQ ID | Complete | GGGAGAGUAUCCGUUGGAGGCAUGCACGAUGUCCAUGACCAACU |
| NO: 56 | sequence of | CCAGUCACGGCCCUGCAGCGUUAGGCUGUAGAUCGGAAGAGCGU |
| | aptamer R02 | CGUGUAGG |
| SEQ ID | Complete | GGGAGAGUAUCCGUUGGAGGCAUCCAGCAUCACCAGCGGCACGA |
| NO: 57 | sequence of | CGUCGGACGGCUGGCUGGUCCGUCACCGUAGAUCGGAAGAGCG |
| | aptamer R03 | UCGUGUAGG |
| SEQ ID | Complete | GGGAGAGUAUCCGUUGGAGGCAUGCAGCUACACGGUGCAAGUAG |
| NO: 58 | sequence of | CACGUCCUGCCAUGCAUGCAGUGCUGCAGAUCGGAAGAGCGUCG |
| | aptamer R04 | UGUAGG |
| SEQ ID | Complete | GGGAGAGUAUCCGUUGGAGGCAUCGGGAAGCAGCACGACGGCCU |
| NO: 59 | sequence of | CAAUGCACUUGCCGGUUGGUUUCGGCUGCAGAUCGGAAGAGCGU |
| | aptamer R05 | CGUGUAGG |
| SEQ ID | Complete | GGGAGAGUAUCCGUUGGAGGCAUCUGCGAAGUGCCCAAGACCAU |
| NO: 60 | sequence of | AUCCACUGCACACGACAGCUGAUGGUGGCAGAUCGGAAGAGCGU |
| | aptamer R84 | CGUGUAGG |
| SEQ ID | Complete | GGGAGAGUAUCCGUUGGAGGCAUGCUACACACACAUCGCACGUC |
| NO: 61 | sequence | ACCCACUAUGGGGACAUCUUGCGGCGUGCAGAUCGGAAGAGCGU |
| | of | CGUGUAGG |
| | aptamer P91 | |

At least one additional aptamer of the composition or of the kit according to the invention may further comprise, in 3′ of the complete sequence “Primer 5′-Additional aptamer specific sequence-Primer 3′ as defined above, a primer sequence 3′ of modified RNA having at least 85% identity, preferably at least 86% identity, preferably at least 87% identity, preferably at least 88% identity, preferably at least 89% identity, preferably at least 90% identity, preferably at least 91% identity, preferably at least 92% identity, preferably at least 93% identity, preferably at least 94% identity, preferably at least 95% identity, preferably at least 96% identity, preferably at least 97% identity, preferably at least 98% identity, preferably at least 99% identity, preferably 100% identity, with SEQ ID NO: 32, preferably located at the end 3′ of the additional aptamer. According to this embodiment, the additional aptamer is in the form “Primer 5′-Specific additional aptamer sequence-Primer 3′-Sequence G” (in the 5′-3′ direction of the modified RNA sequence), this form being named below “complete sequence G of the additional aptamer” (or combined sequence G of the additional aptamer).

According to a preferred embodiment, the RNA of at least one aptamer according to the invention of the composition or of the kit according to the invention was modified in order to increase its resistance to RNA nucleases. Advantageously, the RNA of at least one aptamer according to the invention of the composition or of the kit according to the invention (preferably the RNA of all the aptamers according to the invention of the composition or of the kit according to the invention) was modified by at least one modification chosen from: modification of the OH function on the carbon in the position 2′ of the ribose by methylation; the substitution of the OH function on the carbon in the position 2′ of the ribose by an O-Methoxyethyl group; the substitution of the OH function on the carbon in the position 2′ of the ribose by an amino group; the substitution of the OH function on the carbon in the position 2′ of the ribose by a halogen (in particular by fluorine); the replacement of the phosphodiester (PO) by a phosphorothioate (PS) group (therefore this is referred to as a phosphorothioate skeleton); the use of a Locked Nucleic Acid (LNA) type structure, that is to say the formation of a methylene bridge in order to lock the ribose in the C3′-endo (N-type) conformation; the use of a Peptide Nucleic Acid (PNA) type structure, that is to say the replacement of the sugar-phosphate backbone with a peptide type backbone; and any combination thereof.

The RNA of at least one aptamer according to the invention of the composition or of the kit according to the invention (preferably the RNA of all the aptamers according to the invention of the composition or of the kit according to the invention) has been preferably modified by at least one modification chosen from modification of the OH function on the carbon in the position 2′ of the ribose by methylation; the substitution of the OH function on the carbon in the position 2′ of the ribose by an O-Methoxyethyl group; the substitution of the OH function on the carbon in the position 2′ of the ribose by an amino group; the substitution of the OH function on the carbon in the position 2′ of the ribose by a halogen (in particular by fluorine); and any combination thereof. According to a particularly preferred embodiment, the RNA of at least one aptamer according to the invention of the composition or of the kit according to the invention (preferably the RNA of all the aptamers according to the invention of the composition or of the kit according to the invention) was modified so that the riboses of the pyrimidine nucleotides carry a fluorine atom on the carbon in the position 2′. Thus, preferably, the modified RNA of the aptamer according to the invention of the composition or of the kit according to the invention (preferably the modified RNA of all the aptamers according to the invention of the composition or of the kit according to the invention) is an RNA the riboses of pyrimidine nucleotides of which carry a fluorine atom on the carbon in position 2′, preferably in which the riboses of purine nucleotides are unchanged (it is therefore a 2′Fluoro-pyrimidine RNA (2′F-PyRNA)).

According to a preferred embodiment, the RNA of at least one additional aptamer of the composition or of the kit according to the invention was modified in order to increase its resistance to RNA nucleases. Advantageously, the RNA of at least one additional aptamer of the composition or of the kit according to the invention (preferably the RNA of all the additional aptamers of the composition or of the kit according to the invention) was modified by at least one modification chosen from: the modification of the OH function on the carbon in the position 2′ of the ribose by methylation; the substitution of the OH function on the carbon in the position 2′ of the ribose by an O-Methoxyethyl group; the substitution of the OH function on the carbon in the position 2′ of the ribose by an amino group; the substitution of the OH function on the carbon in the position 2′ of the ribose by a halogen (in particular by fluorine); the replacement of the phosphodiester (PO) by a phosphorothioate (PS) group (therefore this is referred to as a phosphorothioate skeleton); the use of a Locked Nucleic Acid (LNA) type structure, that is to say the formation of a methylene bridge in order to lock the ribose in the C3′-endo (N-type) conformation; the use of a Peptide Nucleic Acid (PNA) type structure, that is to say the replacement of the sugar-phosphate backbone with a peptide type backbone; and any combination thereof.

The RNA of at least one additional aptamer of the composition or of the kit according to the invention (preferably the RNA of all the additional aptamers of the composition or of the kit according to the invention) was preferably modified by at least one modification chosen from the modification of the OH function on the carbon in the position 2′ of the ribose by methylation; the substitution of the OH function on the carbon in the position 2′ of the ribose by an O-Methoxyethyl group; the substitution of the OH function on the carbon in the position 2′ of the ribose by an amino group; the substitution of the OH function on the carbon in the position 2′ of the ribose by a halogen (in particular by fluorine); and any combination thereof. According to a particularly preferred embodiment, the RNA of at least one additional aptamer of the composition or of the kit according to the invention (preferably the RNA of all the additional aptamers of the composition or of the kit according to the invention) was modified so that the riboses of the pyrimidine nucleotides carry a fluorine atom on the carbon in position 2′. Thus, preferably, the modified RNA of the additional aptamer of the composition or of the kit according to the invention (preferably the modified RNA of all the additional aptamers of the composition or of the kit according to the invention) is an RNA the riboses of pyrimidine nucleotides of which carry a fluorine atom on the carbon in position 2′, preferably in which the riboses of purine nucleotides are unchanged (it is therefore a 2′Fluoro-pyrimidine RNA (2′F-PyRNA)).

Advantageously, the RNA of all the additional aptamers of the composition or of the kit according to the invention was modified as defined above. Particularly preferably, the RNA of at least one additional aptamer of the composition or of the kit according to the invention (preferably of all the additional aptamers of the composition or of the kit according to the invention) was modified in the same way as the RNA of at least one aptamer according to the invention of the composition or of the kit according to the invention (preferably of all the aptamers according to the invention of the composition or of the kit according to the invention).

According to a preferred embodiment, the composition or kit according to the invention comprises, or essentially consists of, or consists of, at least the following aptamers:
aptamer comprising a sequence specific for modified RNA having at least 85% identity with SEQ ID NO: 1 (specific sequence of aptamer N30),
aptamer comprising a sequence specific for modified RNA having at least 85% identity with SEQ ID NO: 2 (specific sequence of aptamer N124),
aptamer comprising a sequence specific for modified RNA having at least 85% identity with SEQ ID NO: 3 (specific sequence of aptamer N3),
aptamer comprising a sequence specific for modified RNA having at least 85% identity with SEQ ID NO: 8 (specific sequence of aptamer NO),
aptamer comprising a sequence specific for modified RNA having at least 85% identity, with SEQ ID NO: 9 (specific sequence of aptamer N1),
aptamer comprising a sequence specific for modified RNA having at least 85% identity with SEQ ID NO: 10 (specific sequence of aptamer N2),
aptamer comprising a sequence specific for modified RNA having at least 85% identity with SEQ ID NO: 11 (specific sequence of aptamer N4),
aptamer comprising a sequence specific for modified RNA having at least 85% identity with SEQ ID NO: 12 (specific sequence of aptamer N5),
aptamer comprising a sequence specific for modified RNA having at least 85% identity with SEQ ID NO: 13 (specific sequence of aptamer N15),
aptamer comprising a sequence specific for modified RNA having at least 85% identity with SEQ ID NO: 14 (specific sequence of aptamer N20),
aptamer comprising a sequence specific for modified RNA having at least 85% identity with SEQ ID NO: 16 (specific sequence of aptamer N62),
aptamer comprising a sequence specific for modified RNA having at least 85% identity with SEQ ID NO: 17 (specific sequence of aptamer N73), and
aptamer comprising a sequence specific for modified RNA having at least 85% identity with SEQ ID NO: 18 (specific sequence of aptamer N164);
the kit or composition preferably further comprising at least one aptamer comprising a random sequence of modified RNA.

Indeed, the inventors have shown that with such an assortment of aptamers, a specific molecular fingerprint (a characteristic signature or else a distinctive profile) can be obtained for a biological sample containing different conformers of the α-Syn protein. This specific molecular fingerprint can be used in the diagnostic, prognosis, stratification or monitoring of neurodegenerative diseases, in particular synucleinopathies.

According to a particularly preferred embodiment, the composition or kit according to the invention comprises, or essentially consists of, or consists of, at least the aptamers (that is to say an assortment/mixture of aptamers) comprising a sequence specific for modified RNA having at least 85% identity, preferably at least 86% identity, preferably at least 87% identity, preferably at least 88% identity, preferably at least 89% identity, preferably at least 90% identity, preferably at least 91% identity, preferably at least 92% identity, preferably at least 93% identity, preferably at least 94% identity, preferably at least 95% identity, preferably at least 96% identity, preferably at least 97% identity, preferably at least 98% identity, preferably at least 99% identity, preferably 100% identity, with the following sequences: SEQ ID NO: 1, SEQ ID NO: 2, SEQ ID NO: 3, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 16, SEQ ID NO: 17, and SEQ ID NO: 18;
the kit or composition preferably further comprising at least one aptamer comprising a random sequence of modified RNA.

According to one embodiment, at least one aptamer of the assortment of aptamers of the composition or of the kit according to the invention further comprises:
i. in 5′ of the specific sequence, a primer sequence 5′ of modified RNA having at least 85% identity, preferably at least 86% identity, preferably at least 87% identity, preferably at least 88% identity, preferably at least 89% identity, preferably at least 90% identity, preferably at least 91% identity, preferably at least 92% identity, preferably at least 93% identity, preferably at least 94% identity, preferably at least 95% identity, preferably at least 96% identity, preferably at least 97% identity, preferably at least 98% identity, preferably at least 99% identity, preferably 100% identity, with a sequence chosen from SEQ ID NO: 29 (primer sequence P72 of aptamers N, in 5′) and SEQ ID NO: 30 (primer sequence P73 of aptamers 4F, F, R and P, in 5′), preferably located at the end 5′ of the specific sequence; and/or
ii. in 3′ of the specific sequence, a primer sequence 3′ of modified RNA having at least 85% identity, preferably at least 86% identity, preferably at least 87% identity, preferably at least 88% identity, preferably at least 89% identity, preferably at least 90% identity, preferably at least 91% identity, preferably at least 92% identity, preferably at least 93% identity, preferably at least 94% identity, preferably at least 95% identity, preferably at least 96% identity, preferably at least 97% identity, preferably at least 98% identity, preferably at least 99% identity, preferably 100% identity, with a sequence chosen from SEQ ID NO: 31 (primer PiRT in 3′), SEQ ID NO: 32 (Sequence G of 24 nts in 3′, see page 128 of the thesis) and SEQ ID NO: 33 (PiRT-G; primer PiTR+sequence G combination, in 3′), preferably located at the end 3′ of the specific sequence.

Thus, according to a particularly advantageous embodiment, the composition or kit according to the invention comprises, or essentially consists of, or consists of, at least the aptamers (that is to say an assortment/mixture of aptamers) comprising a complete sequence of modified RNA having at least 85% identity, preferably at least 86% identity, preferably at least 87% identity, preferably at least 88% identity, preferably at least 89% identity, preferably at least 90% identity, preferably at least 91% identity, preferably at least 92% identity, preferably at least 93% identity, preferably at least 94% identity, preferably at least 95% identity, preferably at least 96% identity, preferably at least 97% identity, preferably at least 98% identity, preferably at least less 99% identity, preferably 100% identity, with the following sequences: SEQ ID NO: 34, SEQ ID NO: 35, SEQ ID NO: 36, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, SEQ ID NO: 50, and SEQ ID NO: 51;
the kit or composition preferably further comprising at least one aptamer comprising a random sequence of modified RNA.

At least one aptamer from the assortment of aptamers of the composition or of the kit according to the invention may further comprise, in 3′ of the complete sequence “Primer 5′-Specific aptamer assortment sequence-Primer 3′ as defined above, a primer sequence 3′ of modified RNA having at least 85% identity, preferably at least 86% identity, preferably at least 87% identity, preferably at least 88% identity, preferably at least 89% identity, preferably at least 90% identity, preferably at least 91% identity, preferably at least 92% identity, preferably at least 93% identity, preferably at least 94% identity, preferably at least 95% identity, preferably at least 96% identity, preferably at least 97% identity, preferably at least 98% identity, preferably at least 99% identity, preferably 100% identity, with SEQ ID NO: 32, preferably located at the end 3′ of the aptamer of the assortment. According to this embodiment, the aptamer of the assortment is in the form “Primer 5′-Specific aptamer assortment sequence-Primer 3′-Sequence G” (in the 5′-3′ direction of the modified RNA sequence), this form being named below “complete assortment aptamer sequence G” (or combined assortment aptamer sequence G).

According to one embodiment, the composition or kit according to the invention further comprises at least one aptamer (aptamer called “random aptamer”) comprising, or essentially consisting of, or consisting of, a random sequence of modified RNA having at least 85% identity, preferably at least 86% identity, preferably at least 87% identity, preferably at least 88% identity, preferably at least 89% identity, preferably at least 90% identity, preferably at least 91% identity, preferably at least 92% identity, preferably at least 93% identity, preferably at least 94% identity, preferably at least 95% identity, preferably at least 96% identity, preferably at least 97% identity, preferably at least 98% identity, preferably at least 99% identity, preferably 100% identity, with a sequence chosen from SEQ ID NO: 62 (Scr-1), and SEQ ID NO: 63 (Scr-2), preferably with SEQ ID NO: 63.

Table 6 shows the sequences SEQ ID NO: 62 and SEQ ID NO: 63.

TABLE 6
Sequences of random aptamers Scr-1 and Scr-2.

| SEQ ID | Description | Sequence |
|---|---|---|
| SEQ ID | Random | GGGAGAGUAUCCGUUGGAGGCAUAUCGUUCA |
| NO: 62 | aptamer | GCGUGGGAUCUGCUACAACUCCUGAGUGCUA |
| | sequence | CAUGUACGAGAAGAUCGGAAGAGCGUCGUGU |
| | Scr-1 | AGG |
| SEQ ID | Random | GGGAGAGUAUCCGUUGAGGCUGAUUCAACAC |
| NO: 63 | aptamer | CGUUUGACGUUCUUGGUAUCGGAAGACAGAU |
| | sequenc | CGGAAGAGCGUCGUGUAGG |
| | eScr-2 | |

According to a preferred embodiment, the RNA of at least one random aptamer of the composition or of the kit according to the invention was modified in order to increase its resistance to RNA nucleases. Advantageously, the RNA of at least one random aptamer of the composition or of the kit according to the invention (preferably the RNA of all the random aptamers of the composition or of the kit according to the invention) was modified by at least one modification chosen from: the modification of the OH function on the carbon in the position 2′ of the ribose by methylation; the substitution of the OH function on the carbon in the position 2′ of the ribose by an O-Methoxyethyl group; the substitution of the OH function on the carbon in the position 2′ of the ribose by an amino group; the substitution of the OH function on the carbon in the position 2′ of the ribose by a halogen (in particular by fluorine); the replacement of the phosphodiester (PO) by a phosphorothioate (PS) group (therefore this is referred to as a phosphorothioate skeleton); the use of a Locked Nucleic Acid (LNA) type structure, that is to say the formation of a methylene bridge in order to lock the ribose in the C3′-endo (N-type) conformation; the use of a Peptide Nucleic Acid (PNA) type structure, that is to say the replacement of the sugar-phosphate backbone with a peptide type backbone; and any combination thereof.

The RNA of at least one random aptamer of the composition or of the kit according to the invention (preferably the RNA of all the random aptamers of the composition or of the kit according to the invention) was preferably modified by at least one modification chosen from the modification of the OH function on the carbon in the position 2′ of the ribose by methylation; the substitution of the OH function on the carbon in the position 2′ of the ribose by an O-Methoxyethyl group; the substitution of the OH function on the carbon in the position 2′ of the ribose by an amino group; the substitution of the OH function on the carbon in the position 2′ of the ribose by a halogen (in particular by fluorine); and any combination thereof. According to a particularly preferred embodiment, the RNA of at least one random aptamer of the composition or of the kit according to the invention (preferably the RNA of all the random aptamers of the composition or of the kit according to the invention) was modified so that the riboses of the pyrimidine nucleotides carry a fluorine atom on the carbon in position 2′. Thus, preferably, the modified RNA of the random aptamer of the composition or of the kit according to the invention is an RNA the riboses of pyrimidine nucleotides of which carry a fluorine atom on the carbon in position 2′, preferably in which the riboses of the purine nucleotides are unchanged (it is therefore a 2′Fluoro-pyrimidine RNA (2′F-PyRNA)).

Advantageously, the RNA of all the random aptamers of the composition or of the kit according to the invention was modified as defined above. Particularly preferably, the RNA of at least one random aptamer of the composition or of the kit according to the invention (preferably of all the random aptamers of the composition or of the kit according to the invention) was modified in the same way as the RNA of at least one aptamer according to the invention and/or of at least one additional aptamer (as defined above) of the composition or of the kit according to the invention (preferably of all the aptamers according to the invention and/or all the additional aptamers of the composition or of the kit according to the invention).

According to one embodiment, the kit according to the invention is characterized in that, when the kit comprises several aptamers, said aptamers are:
a) all in one composition, or
b) distributed in several distinct compositions in separate containers, including the case where each of the aptamers is in a distinct composition located in a separate container.

According to one embodiment, the kit according to the invention further comprises instructions for use.

The composition or kit according to the invention may further comprise an excipient (chosen for example from supports, solvents, diluents, adjuvants, dispersion media, coatings, antibacterial and antifungal agents, absorption agents, and any combination thereof), a buffer solution (chosen for example from Tris, Hepes, phosphate, sodium, and any combination thereof), a solution of divalent ions (chosen for example from magnesium ions, calcium, sodium, potassium and any combination thereof), an enzyme (chosen for example from DNA polymerases, RNA polymerases, reverse transcriptases, ligases, and any combination thereof), nucleotides, etc., and any combination thereof. In the case of a kit, the excipients, buffer solutions, divalent ion solution, enzymes, nucleotides, etc., may each be in a distinct composition located in a separate container, or may be mixed in pairs or more in separate compositions in separate containers, or all in the same composition. They can also be mixed with one or more aptamers of the kit.

Non-limiting examples of excipients include water, NaCl, saline solutions, saccharide solutions (for example glucose, trehalose, sucrose, dextrose, etc.), lactated Ringer's, alcohols, oils, gelatins, carbohydrates such as lactose, amylose or starch, fatty acid esters, hydroxymethylcellulose, etc. (see for example the most recent edition of Remington: The Science and Practice of Pharmacy, A. Gennaro, Lippincott, Williams &t Wilkins).

The composition or kit according to the invention may further comprise the compounds necessary for the amplification (in vitro) of the conformers of F-type and/or R-type α-Syn fibers. The amplification of the fiber conformers can be carried out by any technique known to the person skilled in the art. Such techniques comprise in particular protein misfolding cyclic amplification (PMCA) methods, for example as described in Fenyi and al., 2019. Thus, the composition or the kit according to the invention may further comprise a buffer solution adapted for the amplification of the α-Syn fiber conformers (chosen for example from Tris, Tris-HCL, Hepes, KCL, and any combination thereof), and/or α-Syn monomers, etc.

### Uses

In the context of the present invention, the inventors have developed modified ribonucleic acid (RNA) aptamers capable of distinguishing conformers of F-type α-Syn fibers from other α-Syn and α-Syn monomer.

The data show in particular that these aptamers are tools for specific and sensitive detection of different α-Syn fibers. The present invention therefore provides aptamers useful for carrying out screening of molecules but also tools for research in the field of neurodegenerative diseases.

The present invention therefore relates to the in vitro use of at least one aptamer according to the invention, of at least one composition according to the invention, of at least one kit according to the invention, or of any combination thereof, for:
a) detecting the presence or absence of at least one conformer of F-type α-Syn fibers in a biological sample;
b) quantifying (that is to say determining the amount of) a conformer of F-type α-Syn fibers in a biological sample;
c) establishing a molecular fingerprint of α-Syn fiber conformers, preferably F and R type α-Syn fibers of α-Syn, in a biological sample;
d) screening compounds/molecules capable of detecting and/or recognizing a conformer of F-type α-Syn fibers, preferably screening compounds/molecules capable of discriminating the conformers of F-type α-Syn fibers from conformers of R-type α-Syn fibers; or
e) any combination of a) to d).

The in vitro use for a) detecting the presence or absence of at least one conformer of F-type α-Syn fibers in a biological sample preferably comprises the following steps:
1. contacting a biological sample with at least one aptamer according to the invention, at least one composition according to the invention, at least one kit according to the invention, or any combination thereof;
2. detecting the presence or absence of the aptamer fixed in the sample;
3. determining the amount and/or presence or absence of at least one α-Syn conformer, preferably conformers of F-type α-Syn fibers in the biological sample.

Detection methods may in particular use ELISA techniques, gel retardation tests, filtration (in particular filtration on a nitrocellulose membrane), chromatography, thermophoresis, “pull-down” tests, equilibrium dialysis, analytical ultracentrifugation, surface plasmon resonance (SPR), spectroscopic tests, isothermal titration calorimetry (ITC), PCR, quantitative PCR, sequencing (in particular high-throughput sequencing), DNA chips etc.

In vitro use to b) quantify (that is to say determine the amount of) a conformer of F-type α-Syn fibers in a biological sample preferably comprises the following steps:
1. contacting a biological sample with at least one aptamer according to the invention, at least one composition according to the invention, at least one kit according to the invention, or any combination thereof;
2. quantifying the amount of aptamer fixed in the sample;
3. determining the amount of at least one α-Syn conformer, preferably conformers of F-type α-Syn fibers in the biological sample.

The quantification methods can in particular use ELISA techniques, gel delay tests, filtration (in particular filtration on a nitrocellulose membrane), chromatography, thermophoresis, “pull-down” tests, equilibrium dialysis, analytical ultracentrifugation, surface plasmon resonance (SPR), spectroscopic tests, isothermal titration calorimetry (ITC), PCR, quantitative PCR, high-throughput sequencing, DNA chip etc.

The in vitro use for c) establishing a molecular fingerprint (that is to say a characteristic signature or else a distinctive profile) of α-Syn fiber conformers, preferably F and R type α-Syn fibers of α-Syn, preferably comprises the following steps:
1. contacting a biological sample with at least one aptamer according to the invention, at least one composition according to the invention, at least one kit according to the invention, or any combination thereof;
2. quantifying the amount of aptamer fixed in the sample;
3. establishing a molecular fingerprint of α-Syn fiber conformers, preferably F and R type α-Syn fibers of α-Syn in the biological sample.

Methods for establishing a molecular fingerprint of α-Syn conformers, in particular conformers of F-type α-Syn fibers, comprise in particular ELISA techniques, gel retardation tests, filtration (in particular nitrocellulose membrane filtration), chromatography, thermophoresis, “pull-down” tests, equilibrium dialysis, analytical ultracentrifugation, surface plasmon resonance (SPR), spectroscopic tests, isothermal titration calorimetry (ITC), PCR, quantitative PCR, high-throughput sequencing, DNA chip etc.

In the context of the use to establish a molecular fingerprint, preferably a composition or a kit according to the invention comprising at least 2 aptamers, and in particular a composition or a kit comprising an assortment of aptamers as defined above will be chosen.

In vitro use for d) screening compounds/molecules capable of detecting and/or recognizing a conformer of F-type α-Syn fibers preferably comprises the following steps:
1. contacting at least one compound/molecule with a sample of conformers of F-type α-Syn fibers and at least one aptamer according to the invention, at least one composition according to the invention, at least one kit according to the invention, or any combination thereof;
2. quantifying the amount of aptamer fixed in the sample;
3. determining the ability of the compound to bind to conformers of F-type α-Syn fibers (hence to induce a loss of aptamer affinity), based on the quantification of step 2.

The quantification methods can in particular use ELISA techniques, gel delay tests, filtration (in particular filtration on a nitrocellulose membrane), chromatography, thermophoresis, “pull-down” tests, equilibrium dialysis, analytical ultracentrifugation, surface plasmon resonance (SPR), spectroscopic tests, isothermal titration calorimetry (ITC), PCR, quantitative PCR, high-throughput sequencing, DNA chip etc.

According to one embodiment, the in vitro use according to the invention, (that is to say the use as described above for a) detecting the presence or absence of at least one conformer of F-type α-Syn fibers in a biological sample, b) quantifying a conformer of F-type α-Syn fibers in a biological sample, c) establishing a molecular fingerprint of α-Syn fiber conformers in a biological sample, d) screening compounds/molecules capable of detecting and/or recognizing a conformer of F-type α-Syn fibers, and any combination thereof) further comprises a step 1′) carried out before step 1), of amplifying (in vitro) conformers of F-type and/or R-type α-Syn fibers in a biological sample or a sample of conformers of F-type α-Syn fibers. The amplification of the fiber conformers can be produced by any technique known to the person skilled in the art. Such techniques comprise in particular protein misfolding cyclic amplification (PMCA) methods, for example as described in Fenyi and al., 2019.

### Methods

The inventors have shown that the aptamers according to the invention can be used as tools for diagnostic, prognosis, stratification or else monitoring of neurodegenerative diseases, in particular synucleinopathies, or else for evaluating the efficacy of a treatment.

The data show in particular that by using an assortment of aptamers, a specific molecular fingerprint (a characteristic signature or even a distinctive profile) can be obtained for a biological sample containing different conformers of the α-Syn protein. This specific molecular fingerprint allows to distinguish neurodegenerative diseases from each other, in particular synucleinopathies.

The present invention therefore relates to an in vitro method for diagnosing a synucleinopathy in a subject having at least one symptom of neurodegenerative disease, comprising:
a) contacting a biological sample of the subject (called sample (A)) with at least one aptamer according to the invention, at least one composition according to the invention, at least one kit according to the invention, or any combination thereof;
b) detecting the presence or absence of at least one conformer of F-type α-Syn fibers, quantifying the conformers of F-type α-Syn fibers, establishing a molecular fingerprint of α-Syn fiber conformers (preferably F and R type α-Syn fibers of α-Syn), or any combination thereof, in the subject's biological sample; and
c) diagnosing the presence or absence of a synucleinopathy in the subject based on the result of step b).

According to a preferred embodiment, the subject is affected by a synucleinopathy, if the presence of at least one conformer of F-type α-Syn fibers is detected and/or if a molecular fingerprint specific to a synucleinopathy is obtained.

According to one embodiment, the method further comprises a step a′) carried out before step a), of amplifying (in vitro) the conformers of F-type and/or R-type α-Syn fibers in a biological sample from said subject (called sample (A)). The amplification of fiber conformers can be carried out by any technique known to the person skilled in the art. Such techniques comprise in particular protein misfolding cyclic amplification (PMCA) methods, for example as described in Fenyi and al., 2019.

According to an alternative embodiment or in combination with the embodiments described above, the method further comprises a step b′) carried out between step b) and step c), of detecting the presence or absence of at least one conformer of F-type α-Syn fibers, quantifying the conformers of F-type α-Syn fibers, establishing a molecular fingerprint of α-Syn fiber conformers (preferably F and R type α-Syn fibers of α-Syn), or any combination thereof, in one or more biological sample(s) of reference subject(s). The reference subject(s) preferably comprises (comprise) at least one reference subject suffering from a synucleinopathy and optionally at least one healthy reference subject. As a sample from a reference subject suffering from a synucleinopathy, in particular use can be made of a sample from a subject suffering from Parkinson's disease (PD), dementia with Lewy bodies (DLB) or multiple system atrophy (MSA). Several samples from reference subjects suffering from different synucleinopathies can be used in step b′). According to this embodiment, the method can further comprise a step b″) carried out between step b′) and step c), of comparing the detected conformers of F-type α-Syn fibers, quantified conformers of F-type α-Syn fibers, and/or the molecular fingerprint of the conformers of F-type α-Syn fibers obtained, for steps b) and b′). In this case, step c) comprises the diagnostic of synucleinopathy in the subject based on the comparison of step b″). The subject will be diagnosed as suffering from a synucleinopathy if the comparison of step b′) shows that the result of step b) is comparable to that obtained in step b′) for a reference sample of a reference subject suffering from a synucleinopathy (according to the different reference samples, a more precise diagnostic among the synucleinopathies can potentially be made on the same principle), and as not suffering from a synucleinopathy if the result of step b) is comparable to that obtained in step b′) for a reference sample from a healthy reference subject.

Thus, the method according to the invention can comprise steps a′), a), b), and c) as described above, or steps a), b), b′), and c) as described above, or steps a′), a), b), b′), and c) as described above, or steps a), b), b′), b″), and c) as described above, or steps a′), a), b), b′), b″), and c) as described above.

The present invention further relates to an in vitro method for stratification of a synucleinopathy, prognosis of a synucleinopathy, monitoring of a synucleinopathy, or evaluation of the efficacy of a synucleinopathy treatment in a subject suffering from synucleinopathy, comprising:
a) contacting a biological sample of the subject (called sample (A)) with at least one aptamer according to the invention, at least one composition according to the invention, at least one kit according to the invention, or any combination thereof;
b) detecting the presence or absence of at least one conformer of F-type α-Syn fibers, quantifying the conformers of F-type α-Syn fibers, establishing a molecular fingerprint of α-Syn fiber conformers (preferably F and R type α-Syn fibers of α-Syn), or any combination thereof, in the subject's biological sample; and
c) stratifying the synucleinopathy, prognosing the synucleinopathy, monitoring the synucleinopathy, evaluating the efficacy of the treatment of the synucleinopathy, in the subject, based on the result of step b);
   wherein synucleinopathy is preferably selected from Parkinson's disease (PD), dementia with Lewy bodies (DLB) and multiple system atrophy (MSA), more preferably synucleinopathy is DLB.

According to a preferred embodiment, the synucleinopathy is aggravated, or the prognosis of the synucleinopathy is negative, or the synucleinopathy has progressed, or the treatment of the synucleinopathy is ineffective or not very effective, if the presence of at least one conformer of F-type α-Syn fibers is detected and/or if a molecular fingerprint specific to a synucleinopathy is obtained.

According to one embodiment, the method further comprises a step a′) carried out before step a), of amplifying (in vitro) the conformers of F-type and/or R-type α-Syn fibers in a biological sample from said subject (called sample (A)). The amplification of fiber conformers can be carried out by any technique known to the person skilled in the art. Such techniques comprise in particular protein misfolding cyclic amplification (PMCA) methods, for example as described in Fenyi and al., 2019.

According to an alternative embodiment or in combination with the embodiments described above, the in vitro prognosis and/or stratification method further comprises a step b′) carried out between step b) and step c), detecting the presence or absence of at least one conformer of F-type α-Syn fibers, quantifying the conformers of F-type α-Syn fibers, establishing a molecular fingerprint of α-Syn fibers (preferably F and R type α-Syn fibers), or any combination thereof, in one or more biological sample(s) of reference subject(s). The reference subject(s) preferably comprises (comprise) at least one subject suffering from a synucleinopathy with a known stratification prognosis/stage/level (preferably at least one reference subject suffering from a synucleinopathy with a known stratification prognosis/stage/level, the synucleinopathy being the same for the reference subject and for the tested/prognosticated subject) and optionally at least one healthy reference subject. As a sample from a reference subject suffering from a synucleinopathy, in particular use can be made of a sample from a subject suffering from Parkinson's disease (PD), dementia with Lewy bodies (DLB) or multiple system atrophy (MSA). Several samples from reference subjects suffering from different synucleinopathies can be used in step b′). Preferably, several samples from reference subjects suffering from the same synucleinopathy but with known different stratification prognoses/stages/levels can be used in step b′). According to this embodiment, the method can further comprise a step b″) carried out between step b′) and step c), of comparing the detected conformers of F-type α-Syn fibers, quantified conformers of F-type α-Syn fibers, and/or the molecular fingerprint of the conformers of F-type α-Syn fibers obtained, for steps b) and b′). In this case, step c) comprises the prognosis and/or stratification of the synucleinopathy in the subject based on the comparison of step b″). The subject will have a comparable prognosis, and/or will be stratified as being at a stratification stage/level which is comparable to that of a reference subject suffering from the same synucleinopathy at a known stratification prognosis/stage/level, if the comparison of step b′) shows that the result of step b) is comparable to that obtained in step b′) for a reference sample of the reference subject (according to the different reference samples, a prognosis and/or a more precise stratification among the different stages can potentially be done on the same principle). The subject will have a more negative prognosis, and/or will be stratified as being at a more advanced (more serious) stratification stage/level, than a reference subject suffering from the same synucleinopathy at a known stratification prognosis/stage/level or a healthy reference subject, if the result of step b) shows a greater detection of F fibers, or a higher amount of F-type α-Syn fibers, or a molecular fingerprint richer in F fibers, than that obtained in step b′) for a reference sample of the reference subject or the healthy reference subject. Conversely, the subject will have a better prognosis, and/or will be stratified as being at a less advanced (less serious) stratification stage/level, than a reference subject suffering from the same synucleinopathy at a known stratification prognosis/stage/level, if the result of step b) shows a lower detection of F fibers, or a lower amount of F-type α-Syn fibers, or a molecular footprint poorer in F fibers, than that obtained in step b′) for a reference sample of the reference subject.

Thus, the method according to the invention can comprise steps a′), a), b), and c) as described above, or steps a), b), b′), and c) as described above, or steps a′), a), b), b′), and c) as described above, or steps a), b), b′), b″), and c) as described above, or steps a′), a), b), b′), b″), and c) as described above.

According to an alternative embodiment or in combination with the embodiments described above, the in vitro method for stratification of a synucleinopathy, prognosis of a synucleinopathy, monitoring of a synucleinopathy, or evaluation of the efficacy of a synucleinopathy treatment in a subject suffering from a synucleinopathy, further comprises a step b′) carried out between step b) and step c), of detecting the presence or absence of at least one conformer of F-type α-Syn fibers, quantifying the conformers of F-type α-Syn fibers, establishing a molecular fingerprint of α-Syn fiber conformers (preferably F and R type α-Syn fibers of α-Syn), or any combination thereof, in a second biological sample of the subject tested (called sample (B)). Said second sample (B) was preferably obtained/taken after the sample (A), for example during a second visit (the sample (A) then having been obtained during a first visit), preferably said sample (B) having been obtained at least 24 hours after the sample (A), more preferably at least 48 hours after the sample (A), more preferably at least 72 hours after the sample (A), more preferably at least 7 days after the sample (A), more preferably at least 10 days after the sample (A), more preferably at least 15 days after the sample (A), more preferably at least 1 month after the sample (A), more preferably at least 2 months after the sample (A), more preferably at least 3 months after the sample (A), more preferably at least 4 months after the sample (A), more preferably at least 5 months after the sample (A), more preferably at least 6 months after the sample (A), more preferably at least 7 months after the sample (A), more preferably at least 8 months after the sample (A), more preferably at least 9 months after the sample (A), more preferably at least 10 months after the sample (A), more preferably at least 11 months after the sample (A), more preferably at least 12 months after the sample (A). More preferably said sample (B) was obtained between 7 days and 6 months after the sample (A), more preferably said sample (B) having been obtained between 10 days and 5 months after the sample (A), more preferably between 15 days and 4 months, more preferably between 21 days and 3 months, more preferably between 30 and 60, more preferably between 40 and 50 days. According to this embodiment, the method can further comprise a step b″) carried out between step b′) and step c), of comparing the detected conformers of F-type α-Syn fibers, quantified conformers of F-type α-Syn fibers, and/or the molecular fingerprint of the obtained conformers of F-type α-Syn fibers, for steps b) (therefore for sample (A) of the subject) and b′) (therefore for the sample (B) of the subject). In this case, step c) comprises the stratification of the synucleinopathy, the prognosis of the synucleinopathy, the monitoring of the synucleinopathy, the evaluation of the efficacy of the treatment of the synucleinopathy, in the subject, based on the comparison of step b″). The subject will be stratified as being at an unchanged/comparable stratification stage/level, or the subject will have an unchanged/comparable prognosis, or the synucleinopathy will have little or no progress in the subject (will be stable), or the treatment of the synucleinopathy will be reasonably effective, if the comparison of step b′) shows that the result of step b) is comparable to that obtained in step b′).

The subject will be stratified as being at a more advanced (more severe) stratification stage/level, or the subject will have a more negative prognosis, or the synucleinopathy will have progressed (worsened, aggravated) in the subject, or the treatment of the synucleinopathy will be ineffective or not very effective, if the result of step b) shows a greater detection of F fibers, or a higher amount of F-type α-Syn fibers, or a molecular fingerprint richer in F fibers, than that obtained in step b′). Conversely, the subject will be stratified as being at a less advanced stage/level of stratification (less serious), or the subject will have a better prognosis, or the synucleinopathy will have improved (will be less serious, will have receded) in the subject, or the treatment of synucleinopathy will be effective, if the result of step b) shows a lower detection of F fibers, or a lower amount of F-type α-Syn fibers, or a less rich molecular fingerprint in F fibers, than that obtained in step b′).

The method according to the above-mentioned embodiment may also comprise a step a′) carried out before step a), of amplifying (in vitro) the conformers of F-type and/or R-type α-Syn fibers in sample (A) and/or sample (B). The amplification of fiber conformers can be carried out by any technique known to the person skilled in the art. Such techniques comprise in particular protein misfolding cyclic amplification (PMCA) methods, for example as described in Fenyi and al., 2019.

Thus, the method according to the invention can comprise steps a′), a), b), and c) as described above, or steps a), b), b′), and c) as described above, or steps a′), a), b), b′), and c) as described above, or steps a), b), b′), b″), and c) as described above, or steps a′), a), b), b′), b″), and c) as described above.

The examples which follow are intended to illustrate the present invention, and cannot be considered as limiting.

### DESCRIPTION OF FIGURES

FIG. 1: Representative example of barcodes/molecular fingerprints of conformers of F (left panel), R (middle panel) and 91 (right panel) type α-Syn fibers obtained following controlled proteolysis with proteinase K (PK) at different times in minutes (1, 5 or 15 min, indicated at the top of each panel; Landureau and al., 2021).

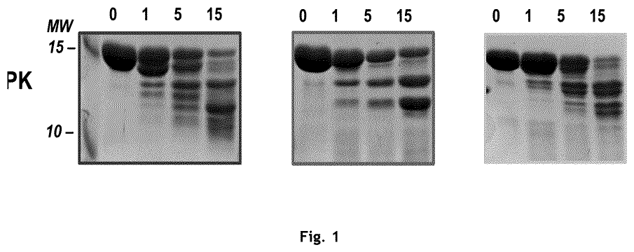

FIG. 2: Results of the screening of the 28 aptamer candidates and their associated random sequences against the F, R, 65 and 91 fibers of α-Syn: Ratio between the amount of oligonucleotide remaining bound on the nitrocellulose membrane relative to the amount of their associated random sequence (Scr1-G or Scr2-G) remaining bound. The graphs show the ratio between the amount of oligonucleotide remaining bound on the nitrocellulose membrane relative to the amount of associated random sequence (Scr1-G or Scr2-G) remaining bound.

FIG. 3: Measurement of the affinity of the aptamer N30-G for F or R type α-Syn fibers.

FIG. 4: Measurement of the affinity of the aptamer N30-G for “amyloid-β” type fibers or the fiber P110 of α-Syn or the α-Syn monomers.

FIG. 5: Measurement of the affinity of the aptamer N124-G for F or R type α-Syn fibers.

FIG. 6: Measurement of the affinity of the aptamer N124-G for “amyloid-β” type fibers or the fiber P110 of α-Syn or the α-Syn monomers.

FIG. 7: Detection of F-type α-Syn fibers by the aptamer N30-G by separation on an Sp6 column.

FIG. 8: Representative example of a molecular fingerprint of the presence of a conformer by analyzing the aptamer frequency of a mixture by high-throughput sequencing.

FIG. 9: Molecular fingerprint of the presence of an F or R conformer in a medium by sequencing the evolution of the frequency of 15 oligonucleotides. A mixture of 14 aptamers (named N) and a control sequence (named Scr-2 (for Scramble 2)) was incubated in a medium containing F or R fiber conformers (respectively conditions Fn1 to Fn3 and Rn1 to Rn3) or in an environment without fiber (conditions 0n1 to 0n4). The figure shows the evolution of the frequency of each aptamer in the mixture compared to its initial frequency in the mixture. This analysis allows to reveal a specific signature for each conformer.

### EXAMPLES

### Example 1: Design and Development of Specific α-Syn Conformer Aptamers

### 1.1. Materials and Methods

### 1.1.1. SELEX

Several SELEXs were performed against different α-Synuclein fiber conformers, in particular the conformers called “F” and “R” conformers. The F fiber conformers have a cylindrical appearance while the R fiber conformers have a ribbon shape. It was shown that rodents can develop two different forms of synucleinopathy: Parkinson's disease and multiple system atrophy, when injected with F or R fiber conformers, respectively.

A selection of aptamers was carried out in RNA chemistry in which all the pyrimidines are modified in the position 2′ of their ribose by a Fluorine group. This chemical modification is known to significantly increase the resistance of RNA to degradation by RNAses.

During these SELEXs, counter-selection steps were carried out to direct the recognition specificity of the aptamers towards a particular conformer type and to ensure that the aptamers have little affinity for the monomeric form of α-Syn. For example, to select aptamers specific for F fiber conformers, a library of 10¹⁵ different oligonucleotides was previously incubated with monomeric forms or R fiber conformers. Only oligonucleotides having no affinity for these conformations were used for a selection against F fiber conformers. With this type of counter selection, the objective was to isolate aptamers having affinity for F fiber conformers but lacking affinity for other forms of the protein.

Samples from the libraries were analyzed by high-throughput sequencing. 28 candidates from these SELEXs were selected (table 7 below). The affinity of these candidates were screened by filtration on nitrocellulose.

### 1.1.2. Affinity Testing of Candidate Aptamers

10 nM of each candidate aptamer (hybridized by heat shock to a radioactively marked oligonucleotide with P32) were incubated with α-Syn fiber conformers at 1 μM in a solution of Ts1X (HEPES (pH 7.6) 10 mM, NaCl 150 mM, KCl 5 mM, CaCl₂) 1.5 mM, MgCl₂ 1 mM) containing Igepal at 0.1% and ssDNA added in a 5-1 proportion relative to the oligonucleotides (that is to say at a level of 1.7 μg/ml). After 30 min of incubation at 37° C., 25 μL of each mixture is deposited in triplicate on the nitrocellulose membrane, then filtered. Two washes with Ts1X are then carried out. The amounts of oligonucleotides retained on the nitrocellulose membrane are quantified by exposing the membrane to a photostimulable phosphorus screen.

1.1.3. Determination of the Dissociation Constants of Candidate Aptamers and Comparisons with Prior Art Aptamers

The affinity of the aptamers was evaluated using a second method, which consists of measuring the amount of complex formed by varying the concentration of the aptamer, and leaving that of the target constant. During these measurements, the target concentration is considered to be so high compared to the aptamer concentration, thus it is possible to neglect the amount of target bound to the aptamer compared to the amount of free target. When the interaction is of the “specific” type between a candidate and its target, the curve of the amount of complex formed as a function of the concentration of candidate initially present must be hyperbolic and show saturation. It is then possible to calculate the affinity constants of the aptamer for its target, the Bₘₐₓ and the K_{d}.

The affinities of the candidate aptamers were also compared to aptamers described in the literature as having been selected against α-syn. aptamers M5-15 and T-S0508, selected by Tsukakoshi and colleagues in 2010 and 2012 respectively (Tsukakoshi and al., 2010, 2012), were chosen. These DNA chemistry aptamers were selected to recognize monomeric and oligomeric forms of α-syn, respectively. It was demonstrated that T-S0508, which recognized α-Syn oligomers with a kd of 68 nM, also had an affinity for Aβ40 oligomers (kd of 25 nM). Also chosen were the aptamers F5R1 and F5R2, which are also in DNA chemistry and were selected in 2019 by Zheng and colleagues (Zheng and al., 2018; Ren and al., 2019). They recognize α-Syn with kd of 2.4 and 3.07 nM respectively. Finally, the DNA chemistry aptamer Tau 3146 was also tested. This aptamer was isolated by a rapid method called “Non-SELEX”, during which three successive rounds of selection without amplification between selections were carried out against the monomeric isoform Tau 441 (Lisi and al., 2018). The aptamer Tau 3146 was shown to be able to bind isoforms Tau 441, Tau 381, Tau 352 and Tau 383 with kds of 13±3 nM, 116±6 nM, 84±6 nM and 49±4 nM, respectively.

As all these aptamers are of DNA chemistry, it is necessary to compare them to a random sequence of the same chemistry and of comparable length. A DNA sequence used in the laboratory as a primer for PCRs was chosen as a control. This primer, named “Scr_DNA” has a size of 87 nucleotides which is comparable to the sequences of the DNA aptamers that were selected from the literature (Table 7 below).

TABLE 7
Sequences of aptamers of the prior art used as a
comparison for the affinity tests of the aptamers
according to the invention and their associated
random sequence.

| Name and | | Length |
|---|---|---|
| SEQ ID NO | Sequence (5′-3′ direction) | (nucleotides) |
| M5-15 | ATAGTCCCATCATTCATTGTATGGTACGGCGCGG | 66 |
| (SEQ ID NO: 65) | TGGCGGGTGCGTGGAGATATTAGCAAGTGTCA | |
| T-SO508 | ATACTGCCATTCATTTCATTTGCCTGTGGTGTTG | 66 |
| (SEQ ID NO: 66) | GGGCGGGTGCGTTTAGATATCAGCATGTGTCA | |
| F5R1 | ATCGAGTGTGTACGGGGTCCGGTAGGGTGGCGA | 58 |
| (SEQ ID NO: 67) | GGTCTTCCTGTCGTAGCAGGATCCA | |
| F5R2 | ATCGAGTGGACGAGTGCCTCCGGTACGAGCTGT | 58 |
| (SEQ ID NO: 68) | CTGATGGGTTTGCGCGCAGGATCCA | |
| Tau 3146 | GCCTGTTGTGAGCCTCCTGTCGAACCTTTGGGG | 76 |
| (SEQ ID NO: 69) | TGGCTTGACGAAGAAAGTAGTTGAGCGTTTATTC | |
| | TTGTCTCCC | |
| Scr_DNA | AATGATACGGCGACCACCGAGATCTACACTCTTT | 58 |
| (SEQ ID NO: 64) | CCCTACACGACGCTCTTCCGATCT | |

The affinity of candidate aptamers as well as that of aptamers from the literature was also measured against different “amyloid” type fibers (Tau1N3R fibers and Aβ40 fibers), in addition to the conformers of F and R α-syn fiber. The affinity of the candidate aptamers against the α-Syn fiber “P110” was also measured. This fiber was made from α-Syn protein truncated from amino acid n° 110 (thus missing the 30 amino acids of the C-terminal domain). The oligonucleotides (aptamers) at 10 nM (hybridized by heat shock to SpG-LNA radioactively marked with P32 for RNA2′F chemistry oligonucleotides, or directly marked with P32 for DNA oligonucleotides) were presented to proteins at 250 nM in a solution of Ts1X containing Igepal at 0.1% and ssDNA added in a 5-1 proportion relative to the oligonucleotides (that is to say between 0.135 μg/ml and 17.325 μg/ml). After 30 min of incubation at 37° C., 25 μL of each mixture is deposited on the nitrocellulose membrane, then filtered. Two washes with Ts1X are then carried out. The amounts of oligonucleotides retained on the nitrocellulose membrane are quantified by exposing the membrane to a photostimulable phosphorus screen. The K_{d} and Bₘₐₓ were calculated by means of the Prism software using a non-linear regression model analyzing the (specific and non-specific) total affinity of a candidate for the target, assuming that there is only one binding site on the target for the candidate. The measurements were carried out in triplicate (three independent experiments on different days) for the F and R type α-Syn fibers, and in duplicate for the other fibers (Tau1N3R and the fibers Aβ40, and P110) as well as for α-Syn monomers.

### 1.1.4. Sp6 Column Affinity Tests

The oligonucleotides radioactively marked with P32 (by hybridization to a SpG-LNA marked with P32 for the 2′F-PyRNA sequences, by direct marking for the DNA sequences) were mixed with the proteins at 250 nM (F fiber or α-Syn monomers) in Ts1X, Igepal 0.1%. After 30 min of incubation at 37° C., 25 μL of the mixtures were deposited on an Sp6 column whose buffer was previously changed into Ts1X. After a first centrifugation of the deposition, two washes with Ts1X of the column were carried out. The columns were finally eluted by washing with 2% SDS solution. The fractions were then deposited in a 24-well plate, and the plate exposed for several hours on a photostimulable phosphor screen. The exposure analysis allowed quantification of each of the fractions for each condition.

### 1.1.5. Molecular Fingerprinting by Analyzing the Aptamer Frequency of a Mixture by High-Throughput Sequencing (FootBal-Seq)

A mixture of 14 aptamers (named N) and a control sequence (named Scr-2 (for Scramble 2)) was incubated in a medium containing F or R fiber conformers (respectively conditions Fn1 to Fn3 and Rn1 to Rn3) or in an environment without fiber (conditions 0n1 to 0n4). The mixture was filtered on an exclusion column and the sequences retained on the column after 3 washes were eluted with 2% SDS. After Phenol-chloroform extraction, the oligonucleotide mixture was amplified by RT-PCR. During this step the sequences were extended by “adaptor” sequences allowing their high-throughput sequencing. After purification by agarose gel electrophoresis, the mixtures were sequenced by high-throughput sequencing.

### 1.2. Results

### 1.2.1. Aptamers Selected by Selex

Table 8 below presents the sequences of the successful candidates.

TABLE 8
Sequences of the selected aptamer candidates.
The random parts (that is to say the specific
parts) of the sequences are underlined, and
the constant parts corresponding to the primer
sites are not underlined. The aptamers Scr-1
and Scr-2 have a random sequence.

| | |
|---|---|
| N0 | GGGAGAGUAUCCGUUGAGGCUGA UCAGUCCAGUACGAGACGCGUUUACCUCCACUGCA |
| | AGAUCGGAAGAGCGUCGUGUAGG |
| N1 | GGGAGAGUAUCCGUUGAGGCUGA AUCCGACCAACCCAACGCGUUUACCUCACCUGCA |
| | AGAUCGGAAGAGCGUCGUGUAGG |
| N2 | GGGAGAGUAUCCGUUGAGGCUGA UUCCGACCGCCGCAACUUAUAGGUAUCCCGCUGCA |
| | AGAUCGGAAGAGCGUCGUGUAGG |
| N3 | GGGAGAGUAUCCGUUGAGGCUGA CAACGCGUUUACCUCACACCACGUCAUCCGUUGCC |
| | AGAUCGGAAGAGCGUCGUGUAGG |
| N4 | GGGAGAGUAUCCGUUGAGGCUGA ACGCGUUUACUCCGCUAGUACGAACCCGAUUGCCC |
| | AGAUCGGAAGAGCGUCGUGUAGG |
| N5 | GGGAGAGUAUCCGUUGAGGCUGA AGCCAUGCUGUCAACUUUUAACUCGUCACCGCUCG |
| | AGAUCGGAAGAGCGUCGUGUAGG |
| N15 | GGGAGAGUAUCCGUUGAGGCUGA AUCGGCCACAGUCGACAACUUUGAAAUCCACCUGC |
| | AGAUCGGAAGAGCGUCGUGUAGG |
| N20 | GGGAGAGUAUCCGUUGAGGCUGA GCAGCACAUGACCUCACCUUUUACUCUGCGCUGCA |
| | AGAUCGGAAGAGCGUCGUGUAGG |
| N30 | GGGAGAGUAUCCGUUGAGGCUGA UCGAUCCACGUCCGACAACGCGUUUACUCGCCAUC |
| | AGAUCGGAAGAGCGUCGUGUAGG |
| N37 | GGGAGAGUAUCCGUUGAGGCUGA UCAGUCCAGCACCAACGCCGUUUGCUCUCGACUAC |
| | AGAUCGGAAGAGCGUCGUGUAGG |
| N62 | GGGAGAGUAUCCGUUGAGGCUGA GUUUCCGAACGGCCCAACUUUGAAAUCCCCGCCCG |
| | AGAUCGGAAGAGCGUCGUGUAGG |
| N73 | GGGAGAGUAUCCGUUGAGGCUGA CAACUUGAAAUCCCAACCCUGCAGCCGUGUCUGGU |
| | AGAUCGGAAGAGCGUCGUGUAGG |
| N124 | GGGAGAGUAUCCGUUGAGGCUGA UCCGACCAUGCUUCAACUUAUACCUCGGGGACUGU |
| | AGAUCGGAAGAGCGUCGUGUAGG |
| N164 | GGGAGAGUAUCCGUUGAGGCUGA GUUUCCGACCACGACCCAACGUUACUGCCCACCAC |
| | AGAUCGGAAGAGCGUCGUGUAGG |
| Scr-2 | GGGAGAGUAUCCGUUGAGGCUGA UUCAACACCGUUUGACGUUCUUGGUAUCGGAAGAC |
| | AGAUCGGAAGAGCGUCGUGUAGG |
| 4F01 | GGGAGAGUAUCCGUUGGAGGCAU |
| | AGCAGCACACGACCAGUGUGCCCCACACCCAGUGGUGGUCUGUGGUGUGC |
| | AGAUCGGAAGAGCGUCGUGUAGG |
| 4F02 | GGGAGAGUAUCCGUUGGAGGCAU |
| | GCUGGCAGCACGCACCGCUGACCGCUGGCUGCACUAUGCGUGUGGAGUGC |
| | AGAUCGGAAGAGCGUCGUGUAGG |
| 4F03 | GGGAGAGUAUCCGUUGGAGGCAU |
| | GCAACAGACGCACCGUACACACAUCUUGGCCGUUGGCUGCCCGACCAGCC |
| | AGAUCGGAAGAGCGUCGUGUAGG |
| 4F04 | GGGAGAGUAUCCGUUGGAGGCAU |
| | CCGUCCACCAGACCAACGUACAAACUCCGCUGGUGGUCGCCUACCCUGGC |
| | AGAUCGGAAGAGCGUCGUGUAGG |
| 4F05 | GGGAGAGUAUCCGUUGGAGGCAU |
| | GCCGCAGGCUACACCACAGCUUCCCCUUCAGCGUGUUGUGGAUACUCGGC |
| | AGAUCGGAAGAGCGUCGUGUAGG |
| R01 | GGGAGAGUAUCCGUUGGAGGCAU |
| | UGCCGCACUACAGCUUGGUCUGCAAUUCCUCUGCGCACAGCUCCAUGUGC |
| | AGAUCGGAAGAGCGUCGUGUAGG |
| R02 | GGGAGAGUAUCCGUUGGAGGCAU |
| | GCACGAUGUCCAUGACCAACUCCAGUCACGGCCCUGCAGCGUUAGGCUGU |
| | AGAUCGGAAGAGCGUCGUGUAGG |
| R03 | GGGAGAGUAUCCGUUGGAGGCAU |
| | CCAGCAUCACCAGCGGCACGACGUCGGACGGCUGGCUGGUCCGUCACCGU |
| | AGAUCGGAAGAGCGUCGUGUAGG |
| R04 | GGGAGAGUAUCCGUUGGAGGCAU |
| | GCAGAGCUACACGGUGCAAGUAGCACGUCCUGCCAUGCAUGCAGUGCUGC |
| | AGAUCGGAAGAGCGUCGUGUAGG |
| R05 | GGGAGAGUAUCCGUUGGAGGCAU |
| | CGGGAAGCAGCACGACGGCCUCAAUGCACUUGCCGGUUGGUUUCGGCUGC |
| | AGAUCGGAAGAGCGUCGUGUAGG |
| Scr-1 | GGGAGAGUAUCCGUUGGAGGCAU |
| | AUCGUUCAGCGUGGGAUCUGCUACAACUCCUGAGUGCUACAUGUACGAGA |
| | AGAUCGGAAGAGCGUCGUGUAGG |
| PF_124 | GGGAGAGUAUCCGUUGGAGGCAU |
| | CACGGACACCUACCCGACGGCAUGUCAGGACACAUGUUGUGCUCCGUGUG |
| | AGAUCGGAAGAGCGUCGUGUAGG |
| R_84 | GGGAGAGUAUCCGUUGGAGGCAU |
| | CUGCGAAGUGCCCAAGACCAUAUCCACUGCACACGACAGCUGAUGGUGGCA |
| | GAUCGGAAGAGCGUCGUGUAGG |
| P65_10 | GGGAGAGUAUCCGUUGGAGGCAU |
| 854 | CGCACAGUGUACACCUACACACAGCAUACCUGUUGGCAUCCCAGGUUGCC |
| | AGAUCGGAAGAGCGUCGUGUAGG |
| P91_70 | GGGAGAGUAUCCGUUGGAGGCAU |
| | GCUACACACACAUCGCACGUCACCCACUAUGGGGACAUCUUGCGGCGUGC |
| | AGAUCGGAAGAGCGUCGUGUAGG |

### 1.2.2. Affinity of Candidate Aptamers for Different α-Syn Conformers

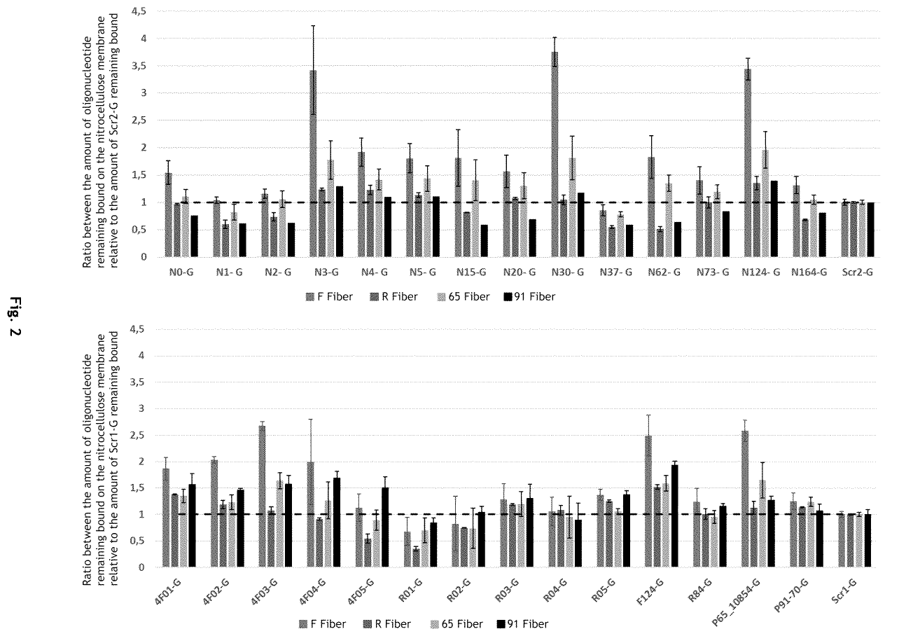

FIG. 2 shows that aptamers N3, N30, N124, 4F01, 4F02, 4F03, 4F05, F124 and P65 remain bound to conformers of F-type α-Syn fibers in much greater amounts than random sequences. In particular, candidates N3-G, N30-G and N124-G remain on average 3.5 times more bound to F-type α-Syn fibers than Scr2-G.

The data further show that aptamers N3, N30, N124, 4F01, 4F02, 4F03, 4F05, F124, and P65 remain bound to the conformers of F-type α-Syn fibers in much greater amounts than to other conformers (R, 91 and 61; FIG. 2). These results demonstrate that aptamers N3, N30, N124, 4F01, 4F02, 4F03, 4F05, F124, and P65 are capable of discriminating conformers of F-type α-Syn fibers from other α-syn conformers.

1.2.3. Determination of the Dissociation Constants of Candidate Aptamers and Comparison with Prior Art Aptamers

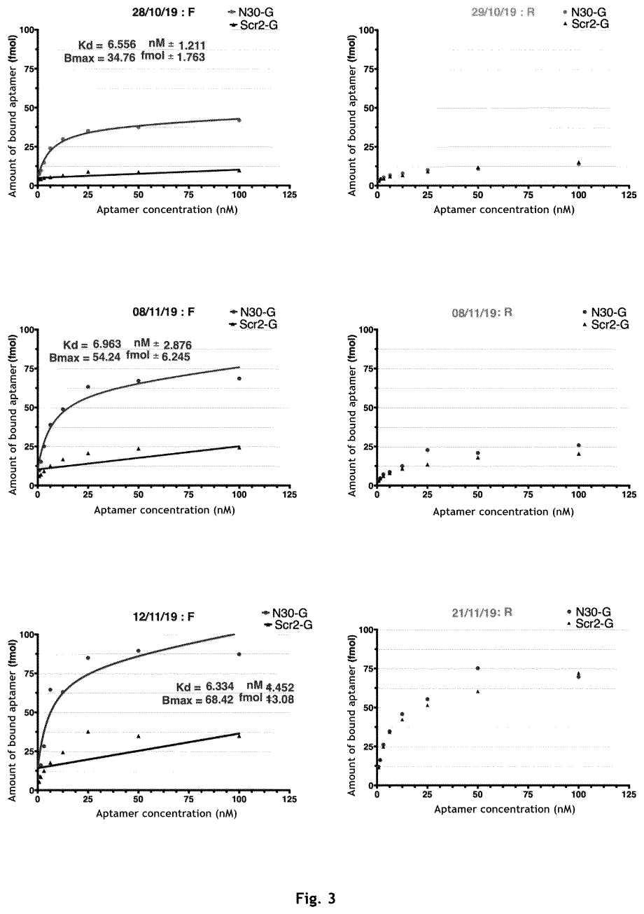

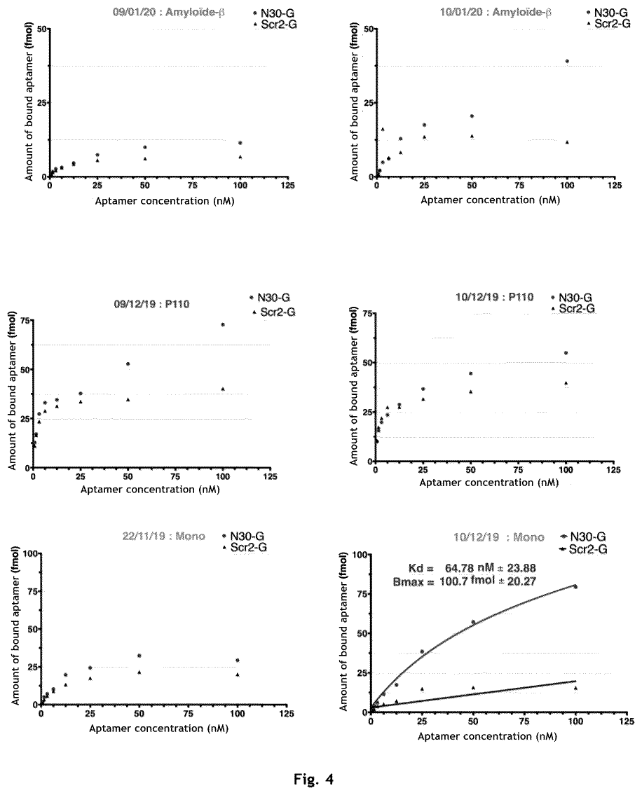

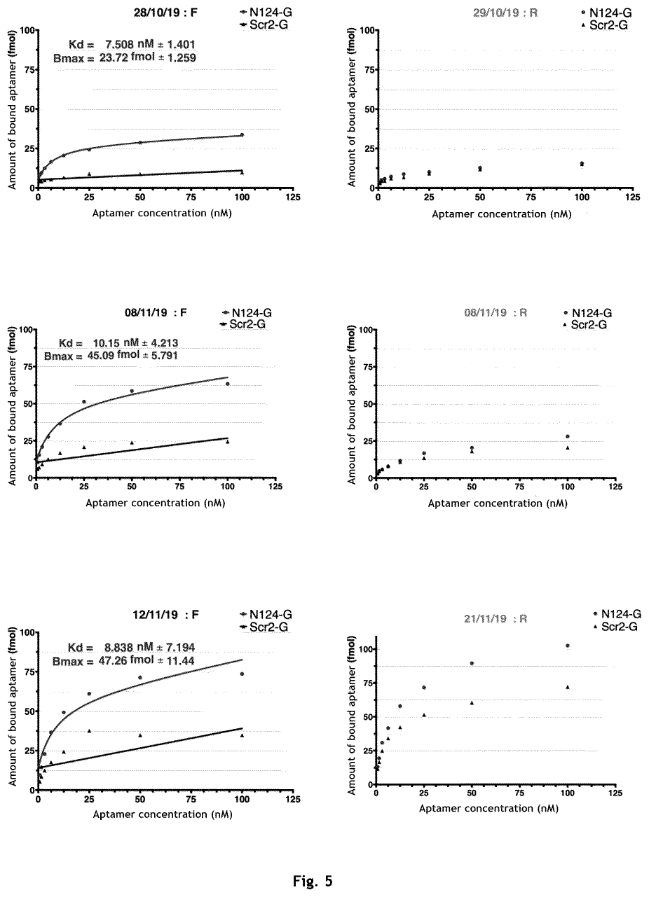

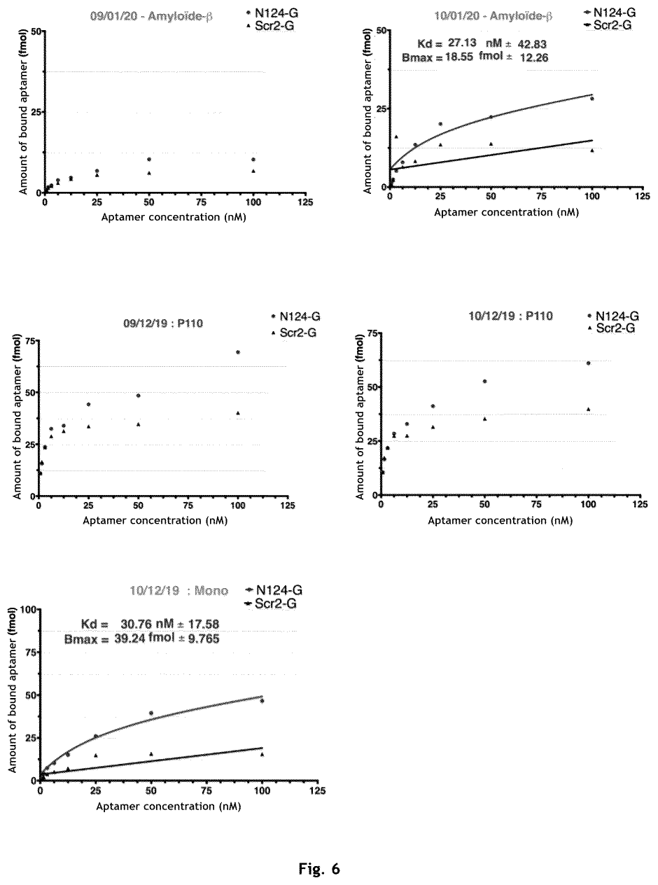

Representative results of each affinity test are shown in FIGS. 3 and 4 for aptamers N30 and in FIGS. 5 and 6 for aptamers N124. Calculations of K_{d} and Bₘₐₓ calculated by Prism are shown on the graphs, when the aptamer was determined to bind significantly to the protein compared to its associated random sequence.

Tables 9 to 12 below summarize the averages of the K_{d} and Bₘₐₓ calculated for each aptamer and for each protein, from all the duplicates or triplicates of experiments carried out, for each type of fiber.

TABLE 9
Average affinity constants (K_{d} and Bₘₐₓ) calculated for the
oligonucleotide andidates, for F-type α-Synuclein fibers.

| | F-type α-Synuclein fiber | | |
|---|---|---|---|
| | K_{d} | Bₘₐₓ | Reproducibility |
| N30-G | 6.62 nM +/− 4.8% | 52.48 fmol +/− 32.2% | 3/3 |
| N124-G | 8.83 nM +/− 14.9% | 38.69 fmol +/− 33.6% | 3/3 |
| T-SO508 | 7.80 nM +/− 4.6% | 20.74 fmol +/− 47.2% | 2/2 |
| M5-15 | No bond | No bond | |
| F5R1 | 3.50 nM +/− 15.8% | 13.66 fmol +/− 28.1% | 2/2 |
| F5R2 | No bond | No bond | |

TABLE 10
Average affinity constants (K_{d} and Bₘₐₓ) calculated for the
oligonucleotide candidates, for R-type α-Synuclein fibers.

| | R-type α-Synuclein fiber | | |
|---|---|---|---|
| | | | Repro- |
| | K_{d} | Bₘₐₓ | ducibility |
| N30-G | No bond | No bond | 3/3 |
| N124-G | No bond | No bond | 3/3 |
| T-SO508 | 18.88 nM +/− 3.8% | 49.95 fmol +/− 102.3% | 2/2 |
| M5-15 | No bond | No bond | |
| F5R1 | 32.41 nM +/− 92.9% | 90.54 fmol +/− 119.0% | 2/2 |
| F5R2 | No bond | No bond | |

TABLE 11
Average affinity constants (K_{d} and Bₘₐₓ) calculated for the
oligonucleotide candidates, for P110 type α-Synuclein fibers.

| | P110 type a-Synuclein fiber | | |
|---|---|---|---|
| | | | Repro- |
| | K_{d} | Bₘₐₓ | ducibility |
| N30-G | No bond | No bond | |
| N124-G | No bond | No bond | |
| T-SO508 | 23.57 nM +/− 59.4% | 48.18 fmol +/− 16.8% | 2/2 |
| M5-15 | — | — | |
| F5R1 | 8.92 nM | 25.04 fmol | 1/2 |
| F5R2 | No bond | No bond | |

TABLE 12
Average affinity constants (K_{d} and Bₘₐₓ) calculated for
the oligonucleotide candidates, for Amyloid β fibers.

| | Amyloid-β | | |
|---|---|---|---|
| | | | Repro- |
| | K_{d} | Bₘₐₓ | ducibility |
| N30-G | No bond | No bond | |
| N124-G | 27.13 nM | 18.55 nM | |
| T-SO508 | 43.21 nM +/− 4.4% | 22.24 fmol +/− 58.1% | 2/2 |
| M5-15 | — | — | |
| F5R1 | 2.54E+25 +/− 141.4% | 3.46E+24 +/− 141.4% | 2/2 |
| F5R2 | No bond | No bond | |

As a percentage, the coefficient of variation is expressed, the ratio between the standard deviation of the values and their average. The “reproducibility” column gives the number of experiments for which binding between the aptamer and the target was measured. The symbol “-” means that no experiment was performed. The word “No binding” is indicated in the table when the binding of the aptamer tested is not significantly greater than that of the random sequence associated therewith.

The data show that measurements of affinities for the F fiber are very reproducible: aptamers N30-G, N124-G, T-S0508 and F5R1 bind to F-type α-Syn fibers with K_{d} less than 10 nM in each experiment. Aptamers T-S0508 and F5R1 also bind to R-type α-Syn fibers in each experiment, while aptamers N30-G and N124-G do not bind to R-type α-Syn fibers in a significantly higher manner compared to their associated random sequences. Aptamers M5-15, F5R2 and Tau 3146 bind neither to F fibers nor to R-type α-Syn fibers (no significantly higher binding than their associated random sequences).

Thus, the data reveal that only the aptamers N30-G and N124-G are capable of discriminating the F fiber from the R fiber among the aptamers tested.

The aptamers of the prior art either recognize the 2 fibers with comparable affinities (aptamers T-S0508 and F5R1), or are not capable of recognizing R-type α-Syn fibers nor F-type α-Syn fibers (M5-15, F5R2 and Tau 3146).

The aptamers T-S0508 and F5R1 have lower K_{d} for the F fiber and higher Bₘₐₓ for the R fiber (differing by a factor of 2). However, a difference in K_{d} of a factor of 2 is not sufficient to allow reliable discrimination between the 2 types of fibers.

FIGS. 4 and 6 further show that aptamers N30 and N124 have no affinity for α-Syn monomers.

Similarly, the data reveal that aptamers N30 and N124 do not significantly recognize amyloid-β fibers (FIGS. 4 and 6 and Table 9).

All these data demonstrate that aptamers N30 and N124 have a highly specific affinity for the F α-Syn fiber. Indeed, the aptamers N30 and N124 are capable of discriminating, in a reproducible and specific manner, the F α-Syn fiber not only from the R α-Syn fiber but also from the α-Syn monomer and other types of fibers such as amyloid-β fibers.

### 1.2.4. Sp6 Column Affinity Tests

The affinity of the aptamer N30-G for F fiber conformers or monomers was tested by filtration on an exclusion column (Sp6 from Biorad). The capabilities of the aptamer N30-G were compared with those of the aptamer T-S0508. The sequences Scr2-G and Scr_DNA were used as controls.

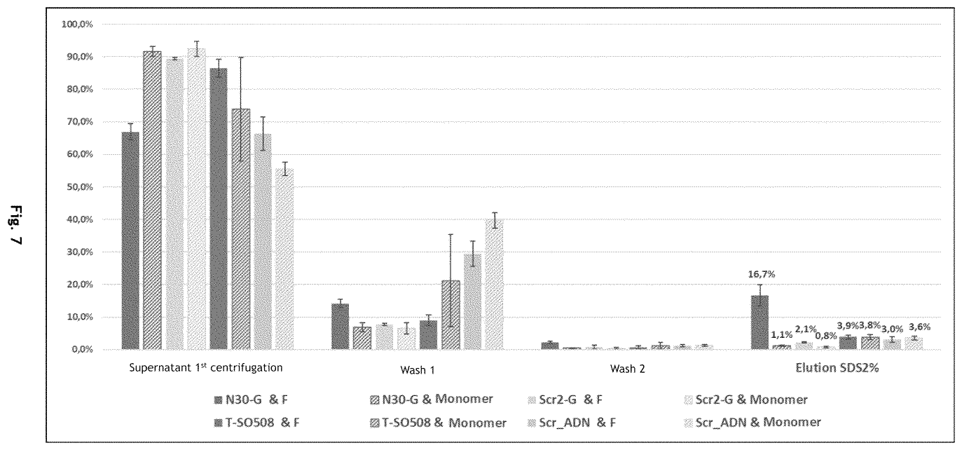

The results of the detection tests are presented in FIG. 7. The data shows that these results are reproducible. It is observed that when the oligonucleotides were presented to the F-type α-Syn fibers, 16.7% of the N30-G, 2.1% of the Scr2-G, 3.9% of the T-S0508 and 3.0% of the Scr_DNA are eluted in the fraction of washing the Sp6 columns with 2% SDS. This means that N30-G is approximately 8 times better retained to F-type α-Syn fibers than its associated random sequence, and 4 times better retained than T-S0508. These results are a further confirmation of the ability of N30-G to recognize F fibers, and also of its superiority compared to T-S0508.

The data show that, when N30-G was presented to the monomers, only 1.1% of the oligonucleotides eluted in the 2% SDS elution fraction. This means that N30-G recognizes F-type α-Syn fibers approximately 16 times better than monomers. Interestingly, the fractions eluted by 2% SDS are similar for T-S0508 whether it was presented to F-type α-Syn fibers or to monomers (3.9 and 3.8% respectively). These results show that T-S0508 does not distinguish between F-type α-Syn fibers and monomers.

### 1.2.5. Obtaining a Diagnostic Molecular Fingerprint by FootBal-Seq

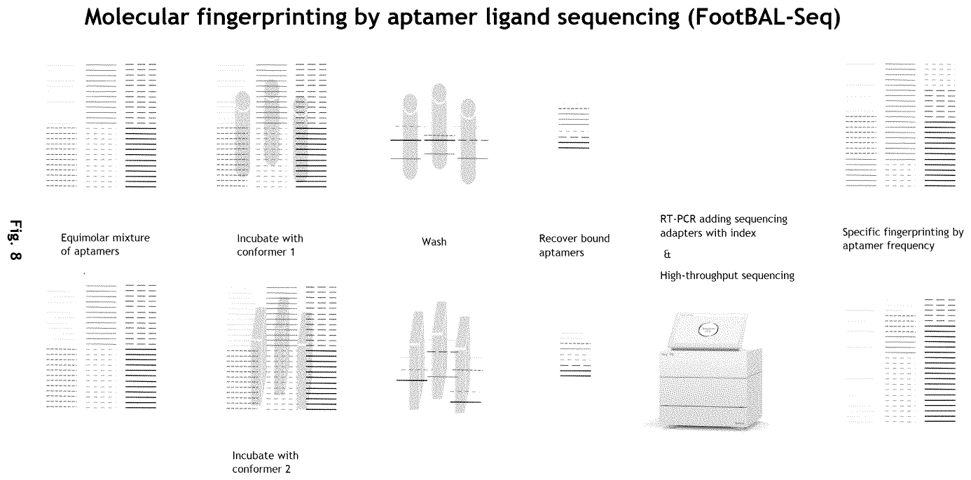

The inventors have developed a new diagnostic method using a mixture of aptamers to diagnose the presence of a fiber conformer in a medium (FIG. 8). A selection of 14 of the 28 aptamers developed was mixed in equimolar amount, as well as a control sequence. This mixture was incubated in a medium containing no fiber or containing Fiber conformers either F or R. The mixtures were then deposited on an Sp6 exclusion column. After amplification and purification, the mixtures were sequenced by high-throughput sequencing. The proportion of each sequence in the mixture is compared to the starting proportion.

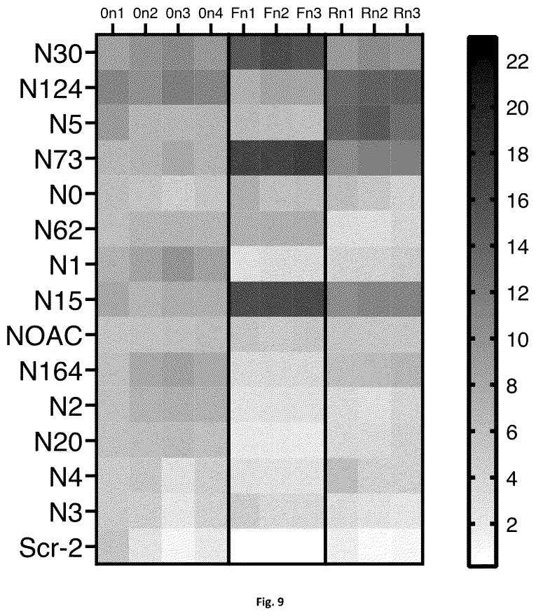

FIG. 9 shows the evolution of the frequency of each aptamer in the mixture compared to its initial frequency in the mixture. This analysis allows to reveal a specific signature for each conformer. For example, a strong enrichment in N30, N73 and N15 compared to the other sequences for the F Fiber conformer and an enrichment in sequences N30, N124, N5, N73 and N15 for the R Fiber conformer.

Thus, the data highlight a specific signature for F fiber conformers and R fiber conformers (FIG. 9).

These results were confirmed in patient samples. These data confirm that such a mixture of aptamers can be used to obtain a signature from patient biological samples, thereby enabling the diagnostic of neurodegenerative diseases. This mixture allows in particular to determine whether the patient suffers from a synucleinopathy or another type of neurodegenerative disease. In the case where it is a synucleinopathy, it is also possible to determine which one, by the same test.

### REFERENCES

Agrawal, M., and Biswas, A. (2015). Molecular diagnostics of neurodegenerative disorders. Front MoL Biosci 2, 54.
Beach, T. G. (2017). A Review of Biomarkers for Neurodegenerative Disease: Will They Swing Us Across the Valley? Neurol. Ther. 6, 5-13.
Bousset, L., Pieri, L., Ruiz-Arlandis, G., Gath, J., Jensen, P. H., Habenstein, B., Madiona, K., Olieric, V., Bockmann, A., Meier, B. H., and al. (2013). Structural and functional characterization of two alpha-synuclein strains. Nat Commun 4, 2575.
Cheng, H.-C., Ulane, C. M., and Burke, R. E. (2010). Clinical Progression in Parkinson's Disease and the Neurobiology of Axons. Ann. Neurol. 67, 715-725.
Fenyi, A., Duyckaerts, C., Bousset, L., Braak, H., Tredici, K. D., Melki, R., and on behalf of the Brainbank Neuro-CEB Neuropathology Network (2021). Seeding Propensity and Characteristics of Pathogenic αSyn Assemblies in Formalin-Fixed Human Tissue from the Enteric Nervous System, Olfactory Bulb, and Brainstem in Cases Staged for Parkinson's Disease. Cells 10, 139.
Fenyi, A., Leclair-Visonneau, L., Clairembault, T., Coron, E., Neunlist, M., Melki, R., (2019). Detection of alpha-synuclein aggregates in gastrointestinal biopsies by protein misfolding cyclic amplification, Neurobiology of Disease 129, 38-43.
Gómez-Río, M., Caballero, M. M., Górriz Sáez, J. M., and Mínguez-Castellanos, A. (2016). Diagnosis of Neurodegenerative Diseases: The Clinical Approach. Curr. Alzheimer Res. 13, 469-474.
Landureau, M., Redeker, V., Bellande, T., Eyquem, S., and Melki, R. (2021). The differential solvent exposure of N-terminal residues provides ‘fingerprints’ of alpha-synuclein fibrillar polymorphs. J. Biol. Chem. 0, https://doi.org/10.1016/j.jbc.2021.100737.
Katsuno, M., Sahashi, K., Iguchi, Y., and Hashizume, A. (2018). Preclinical progression of neurodegenerative diseases. Nagoya J Med Sci 80, 289-298.
Makky, A., Bousset, L., Polesel-Maris, J., and Melki, R. (2016). Nanomechanical properties of distinct fibrillar polymorphs of the protein alpha-synuclein. Sci Rep 6.
Peelaerts, W., Bousset, L., Van der Perren, A., Moskalyuk, A., Pulizzi, R., Giugliano, M., Van den Haute, C., Melki, R., and Baekelandt, V. (2015). α-Synuclein strains cause distinct synucleinopathies after local and systemic administration. Nature 522, 340-344.
Perrin, R. J., Payton, J. E., Barnett, D. H., Wraight, C. L., Woods, W. S., Ye, L., and George, J. M. (2003). Epitope mapping and specificity of the anti-α-synuclein monoclonal antibody Syn-1 in mouse brain and cultured cell lines. Neurosci. Lett. 349, 133-135.
Pieri, L., Madiona, K., and Melki, R. (2016). Structural and functional properties of prefibrillar α-synuclein oligomers. Sci. Rep. 6.
Ren, X., Zhao, Y., Xue, F., Zheng, Y., Huang, H., Wang, W., Chang, Y., Yang, H., and Zhang, J. (2019). Exosomal DNA Aptamer Targeting alpha-Synuclein Aggregates Reduced Neuropathological Deficits in a Mouse Parkinson's Disease Model. Mol Ther Nucleic Acids 17, 726-740.
Rey, N. L., Bousset, L., George, S., Madaj, Z., Meyerdirk, L., Schulz, E., Steiner, J. A., Melki, R., and Brundin, P. (2019). alpha-Synuclein conformational strains spread, seed and target neuronal cells differentially after injection into the olfactory bulb. Acta Neuropathol Commun 7, 221.
Tsukakoshi, K., Harada, R., Sode, K., and Ikebukuro, K. (2010). Screening of DNA aptamer which binds to alpha-synuclein. Biotechnol Lett 32, 643-648.
Vaikath, N. N., Majbour, N. K., Paleologou, K. E., Ardah, M. T., van Dam, E., van de Berg, W. D. J., Forrest, S. L., Parkkinen, L., Gai, W.-P., Hattori, N., and al. (2015). Generation and characterization of novel conformation-specific monoclonal antibodies for α-synuclein pathology. Neurobiol. Dis. 79, 81-99.
Vaikath, N. N., Hmila, I., Gupta, V., Erskine, D., Ingelsson, M., and El-Agnaf, O. M. A. (2019). Antibodies against alpha-synuclein: tools and therapies. J Neurochem 150, 612-625.
Van der Perren, A., Gelders, G., Fenyi, A., Bousset, L., Brito, F., Peelaerts, W., Van den Haute, C., Gentleman, S., Melki, R., and Baekelandt, V. (2020). The structural differences between patient-derived α-synuclein strains dictate characteristics of Parkinson's disease, multiple system atrophy and dementia with Lewy bodies. Acta Neuropathol. (Berl.) 139, 977-1000.
Zheng, Y., Qu, J., Xue, F., Zheng, Y., Yang, B., Chang, Y., Yang, H., and Zhang, J. (2018). Novel DNA Aptamers for Parkinson's Disease Treatment Inhibit alpha-Synuclein Aggregation and Facilitate its Degradation. MoL Ther Nucleic Acids 11, 228-242.

1. An aptamer characterized in that it has the ability to distinguish conformers of F-type α-Syn fibers of the α-Syn protein (α-Syn) from conformers of R-type α-Syn fibers, and in that it comprises a sequence specific for modified ribonucleic acid (RNA) having at least 85% identity with a sequence selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7.

2. The aptamer according to claim 1, whose dissociation constant K_{d(F)} measured for the conformers of F-type α-Syn fibers is:
a) lower than the dissociation constant K_{d(R)} measured for the conformers of R-type α-Syn fibers;
b) lower than the dissociation constant K_{d(Mono)} measured for α-Syn monomers;
c) lower than the dissociation constant K_{d(Random)} of a random aptamer measured for the conformers of F-type α-Syn fibers;
d) lower than the dissociation constant K_{d(R)} measured for the conformers of R-type α-Syn fibers, and lower than the dissociation constant K_{d(Mono)} measured for α-Syn monomers;
e) lower than the dissociation constant K_{d(R)} measured for the conformers of R-type α-Syn fibers, and lower than the dissociation constant K_{d(Random)} of a random aptamer measured for the conformers of F-type α-Syn fibers; or
f) lower than the dissociation constant K_{d(R)} measured for the conformers of R-type α-Syn fibers, lower than the dissociation constant K_{d(mono)} measured for α-Syn monomers, and lower than the dissociation constant K_{d(Random)} of a random aptamer measured for the conformers of F-type α-Syn fibers.

3. The aptamer according to claim 1, having at least one dissociation constant K_{d} as follows:
a) the dissociation constant K_{d(F)} measured for the conformers of F-type α-Syn fibers is less than 15 nM; and/or
b) the dissociation constant K_{d(R)} measured for the conformers of R-type α-Syn fibers is greater than 100 nM.

4. The aptamer according to claim 1, further comprising:
i. in 5′ of the specific sequence, a modified RNA primer sequence having at least 85% identity with a sequence selected from the group consisting of SEQ ID NO: 29 and SEQ ID NO: 30; and/or
ii. in 3′ of the specific sequence, a modified RNA primer sequence having at least 85% identity with a sequence selected from the group consisting of SEQ ID NO: 31, SEQ ID NO: 32 and SEQ ID NO: 33.

5. The aptamer according to claim 1, comprising a modified RNA sequence having at least 85% identity with a sequence selected from the group consisting of SEQ ID NO: 34, SEQ ID NO: 35, SEQ ID NO: 36, SEQ ID NO: 37, SEQ ID NO: 38, SEQ ID NO: 39, and SEQ ID NO: 40.

6. A kit comprising at least one aptamer according to claim 1.

7. The kit according to claim 6, further comprising at least one additional aptamer chosen from aptamers comprising a sequence specific for modified RNA having at least 85% identity with a sequence selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28.

8. The kit according to claim 7, wherein at least one additional aptamer further comprises:
i. in 5′ of the specific sequence, a modified RNA primer sequence having at least 85% identity with a sequence selected from the group consisting of SEQ ID NO: 29 and SEQ ID NO: 30; and/or
ii. 3′ of the specific sequence, a modified RNA primer sequence having at least 85% identity with a sequence selected from the group consisting of SEQ ID NO: 31, SEQ ID NO: 32 and SEQ ID NO: 33.

9. The kit according to claim 6, wherein at least one additional aptamer is chosen from aptamers comprising a modified RNA sequence having at least 85% identity with a sequence selected from the group consisting of SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48 and SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, and SEQ ID NO: 60.

10. The kit according to claim 6, comprising at least the following aptamers:
aptamer comprising a sequence specific for modified RNA having at least 85% identity with SEQ ID NO: 1,
aptamer comprising a sequence specific for modified RNA having at least 85% identity with SEQ ID NO: 2,
aptamer comprising a sequence specific for modified RNA having at least 85% identity with SEQ ID NO: 3,
aptamer comprising a sequence specific for modified RNA having at least 85% identity with SEQ ID NO: 8,
aptamer comprising a sequence specific for modified RNA having at least 85% identity with SEQ ID NO: 9,
aptamer comprising a sequence specific for modified RNA having at least 85% identity with SEQ ID NO: 10,
aptamer comprising a sequence specific for modified RNA having at least 85% identity with SEQ ID NO: 11,
aptamer comprising a sequence specific for modified RNA having at least 85% identity with SEQ ID NO: 12,
aptamer comprising a sequence specific for modified RNA having at least 85% identity with SEQ ID NO: 13,
aptamer comprising a sequence specific for modified RNA having at least 85% identity with SEQ ID NO: 14,
aptamer comprising a sequence specific for modified RNA having at least 85% identity with SEQ ID NO: 16,
aptamer comprising a sequence specific for modified RNA having at least 85% identity with SEQ ID NO: 17, and
aptamer comprising a sequence specific for modified RNA having at least 85% identity with SEQ ID NO: 18.

11. The kit according to claim 6, wherein, when the kit comprises several aptamers, said aptamers are:
a) all in one composition, or
b) distributed in several distinct compositions in separate containers, including the case where each of the aptamers is in a distinct composition located in a separate container.

12. The aptamer according to claim 1, in which the RNA of the aptamer was modified in order to increase its resistance to RNA nucleases.

13. An in vitro method for:
a) detecting the presence or absence of at least one conformer of F-type α-Syn fibers in a biological sample;
b) determining the amount of a conformer of F-type α-Syn fibers in a biological sample;
c) establishing a molecular fingerprint of α-Syn fiber conformers, preferably F and R type α-Syn fibers of α-Syn, in a biological sample;
d) screening compounds/molecules capable of detecting and/or recognizing a conformer of F-type α-Syn fibers, preferably screening compounds/molecules capable of discriminating the conformers of F-type α-Syn fibers from conformers of R-type α-Syn fibers; or
e) any combination of a) to d);
using at least one aptamer according to claim 1, or at least one kit comprising at least one aptamer according to claim 1, or any combination thereof.

14. An in vitro method for diagnosing a synucleinopathy in a subject having at least one symptom of neurodegenerative disease, comprising:
a) contacting a biological sample of the subject with at least one aptamer according to claim 1, or at least one kit comprising at least one aptamer according to claim 1, or with any combination thereof;
b) detecting the presence or absence of at least one conformer of F-type α-Syn fibers, quantifying the conformers of F-type α-Syn fibers, establishing a molecular fingerprint of α-Syn fiber conformers (preferably F and R type α-Syn fibers of α-Syn), or any combination thereof, in the subject's biological sample; and
c) diagnosing the presence or absence of a synucleinopathy in the subject based on the result of step b).

15. An in vitro method for stratification of a synucleinopathy, prognosis of a synucleinopathy, monitoring of a synucleinopathy, or evaluation of the efficacy of a synucleinopathy treatment in a subject suffering from a synucleinopathy, comprising:
a) contacting a biological sample of the subject with at least one aptamer according to claim 1, or at least one kit comprising at least one aptamer according to claim 1, or with any combination thereof;
b) detecting the presence or absence of at least one conformer of F-type α-Syn fibers, quantifying the conformers of F-type α-Syn fibers, establishing a molecular fingerprint of α-Syn fiber conformers (preferably F and R type α-Syn fibers of α-Syn), or any combination thereof, in the subject's biological sample; and
c) stratifying the synucleinopathy, prognosing the synucleinopathy, monitoring the synucleinopathy, evaluating the efficacy of the treatment of the synucleinopathy, in the subject, based on the result of step b).

16. The kit according to claim 6, in which the RNA of all the aptamers of the kit was modified in order to increase its resistance to RNA nucleases.


SEQUENCE LISTING (69 sequences)

SEQ ID NO: 1 - RNA, 35 nt, Artificial
  Synthesized sequence of aptamer N30
ucgauccacguccgacaacgcguuuacucgccauc

SEQ ID NO: 2 - RNA, 35 nt, Artificial
  Synthesized sequence of aptamer N124
uccgaccaugcuucaacuuauaccucggggacugu

SEQ ID NO: 3 - RNA, 35 nt, Artificial
  Synthesized sequence of aptamer N3
caacgcguuuaccucacaccacgucauccguugcc

SEQ ID NO: 4 - RNA, 50 nt, Artificial
  Synthesized sequence of aptamer 4F02
gcuggcagcacgcaccgcugaccgcuggcugcacuaugcguguggagugc

SEQ ID NO: 5 - RNA, 50 nt, Artificial
  Synthesized sequence of aptamer 4F03
gcaacagacgcaccguacacacaucuuggccguuggcugcccgaccagcc

SEQ ID NO: 6 - RNA, 50 nt, Artificial
  Synthesized sequence of aptamer F124
cacggacaccuacccgacggcaugucaggacacauguugugcuccgugug

SEQ ID NO: 7 - RNA, 50 nt, Artificial
  Synthesized sequence of aptamer P65
cgcacaguguacaccuacacacagcauaccuguuggcaucccagguugcc

SEQ ID NO: 8 - RNA, 35 nt, Artificial
  Synthesized sequence of aptamer N0
ucaguccaguacgagacgcguuuaccuccacugca

SEQ ID NO: 9 - RNA, 34 nt, Artificial
  Synthesized sequence of aptamer N1
auccgaccaacccaacgcguuuaccucaccugca

SEQ ID NO: 10 - RNA, 35 nt, Artificial
  Synthesized sequence of aptamer N2
uuccgaccgccgcaacuuauagguaucccgcugca

SEQ ID NO: 11 - RNA, 35 nt, Artificial
  Synthesized sequence of aptamer N4
acgcguuuacuccgcuaguacgaacccgauugccc

SEQ ID NO: 12 - RNA, 35 nt, Artificial
  Synthesized sequence of aptamer N5
agccaugcugucaacuuuuaacucgucaccgcucg

SEQ ID NO: 13 - RNA, 35 nt, Artificial
  Synthesized sequence of aptamer N15
aucggccacagucgacaacuuugaaauccaccugc

SEQ ID NO: 14 - RNA, 35 nt, Artificial
  Synthesized sequence of aptamer N20
gcagcacaugaccucaccuuuuacucugcgcugca

SEQ ID NO: 15 - RNA, 35 nt, Artificial
  Synthesized sequence of aptamer N37
ucaguccagcaccaacgccguuugcucucgacuac

SEQ ID NO: 16 - RNA, 35 nt, Artificial
  Synthesized sequence of aptamer N62
guuuccgaacggcccaacuuugaaauccccgcccg

SEQ ID NO: 17 - RNA, 35 nt, Artificial
  Synthesized sequence of aptamer N73
caacuugaaaucccaacccugcagccgugucuggu

SEQ ID NO: 18 - RNA, 35 nt, Artificial
  Synthesized sequence of aptamer N164
guuuccgaccacgacccaacguuacugcccaccac

SEQ ID NO: 19 - RNA, 50 nt, Artificial
  Synthesized sequence of aptamer 4F01
agcagcacacgaccagugugccccacacccagugguggucuguggugugc

SEQ ID NO: 20 - RNA, 50 nt, Artificial
  Synthesized sequence of aptamer 4F04
ccguccaccagaccaacguacaaacuccgcugguggucgccuacccuggc

SEQ ID NO: 21 - RNA, 50 nt, Artificial
  Synthesized sequence of aptamer 4F05
gccgcaggcuacaccacagcuuccccuucagcguguuguggauacucggc

SEQ ID NO: 22 - RNA, 50 nt, Artificial
  Synthesized sequence of aptamer R01
ugccgcacuacagcuuggucugcauauccucugcgcacagcuccaugugc

SEQ ID NO: 23 - RNA, 50 nt, Artificial
  Synthesized sequence of aptamer R02
gcacgauguccaugaccaacuccagucacggcccugcagcguuaggcugu

SEQ ID NO: 24 - RNA, 50 nt, Artificial
  Synthesized sequence of aptamer R03
ccagcaucaccagcggcacgacgucggacggcuggcugguccgucaccgu

SEQ ID NO: 25 - RNA, 50 nt, Artificial
  Synthesized sequence of aptamer R04
gcagagcuacacggugcaaguagcacguccugccaugcaugcagugcugc

SEQ ID NO: 26 - RNA, 50 nt, Artificial
  Synthesized sequence of aptamer R05
cgggaagcagcacgacggccucaaugcacuugccgguugguuucggcugc

SEQ ID NO: 27 - RNA, 50 nt, Artificial
  Synthesized sequence of aptamer R84
cugcgaagugcccaagaccauauccacugcacacgacagcugaugguggc

SEQ ID NO: 28 - RNA, 50 nt, Artificial
  Synthesized sequence of aptamer P91
gcuacacacacaucgcacgucacccacuauggggacaucuugcggcgugc

SEQ ID NO: 29 - RNA, 23 nt, Artificial
  Synthesized Sequence primer P72 of aptamers N, in 5′
gggagaguauccguugaggcuga

SEQ ID NO: 30 - RNA, 23 nt, Artificial
  Synthesized Sequence primer P73 of aptamers 4F, F, R and P, in 5′
gggagaguauccguuggaggcau

SEQ ID NO: 31 - RNA, 23 nt, Artificial
  Synthesized Sequence primer PiRT in 3′
agaucggaagagcgucguguagg

SEQ ID NO: 32 - RNA, 24 nt, Artificial
  Synthesized Sequence G of 24 nts in 3′
gcauugccccagcgugacugccua

SEQ ID NO: 33 - RNA, 47 nt, Artificial
  Synthesized Sequence PiRT-G  combination primer PiTR + sequence G, in 3′
agaucggaagagcgucguguagggcauugccccagcgugacugccua

SEQ ID NO: 34 - RNA, 81 nt, Artificial
  Synthesized sequence of aptamer N30
gggagaguauccguugaggcugaucgauccacguccgacaacgcguuuacucgccaucagaucggaagagcgucguguagg

SEQ ID NO: 35 - RNA, 81 nt, Artificial
  Synthesized sequence of aptamer N124
gggagaguauccguugaggcugauccgaccaugcuucaacuuauaccucggggacuguagaucggaagagcgucguguagg

SEQ ID NO: 36 - RNA, 81 nt, Artificial
  Synthesized sequence of aptamer N3
gggagaguauccguugaggcugacaacgcguuuaccucacaccacgucauccguugccagaucggaagagcgucguguagg

SEQ ID NO: 37 - RNA, 96 nt, Artificial
  Synthesized sequence of aptamer 4F02
gggagaguauccguuggaggcaugcuggcagcacgcaccgcugaccgcuggcugcacuaugcguguggagugcagaucggaagagcgucguguagg

SEQ ID NO: 38 - RNA, 96 nt, Artificial
  Synthesized sequence of aptamer 4F03
gggagaguauccguuggaggcaugcaacagacgcaccguacacacaucuuggccguuggcugcccgaccagccagaucggaagagcgucguguagg

SEQ ID NO: 39 - RNA, 96 nt, Artificial
  Synthesized sequence of aptamer F124
gggagaguauccguuggaggcaucacggacaccuacccgacggcaugucaggacacauguugugcuccgugugagaucggaagagcgucguguagg

SEQ ID NO: 40 - RNA, 96 nt, Artificial
  Synthesized sequence of aptamer P65
gggagaguauccguuggaggcaucgcacaguguacaccuacacacagcauaccuguuggcaucccagguugccagaucggaagagcgucguguagg

SEQ ID NO: 41 - RNA, 81 nt, Artificial
  Synthesized sequence of aptamer N0
gggagaguauccguugaggcugaucaguccaguacgagacgcguuuaccuccacugcaagaucggaagagcgucguguagg

SEQ ID NO: 42 - RNA, 80 nt, Artificial
  Synthesized sequence of aptamer N1
gggagaguauccguugaggcugaauccgaccaacccaacgcguuuaccucaccugcaagaucggaagagcgucguguagg

SEQ ID NO: 43 - RNA, 81 nt, Artificial
  Synthesized sequence of aptamer N2
gggagaguauccguugaggcugauuccgaccgccgcaacuuauagguaucccgcugcaagaucggaagagcgucguguagg

SEQ ID NO: 44 - RNA, 81 nt, Artificial
  Synthesized sequence of aptamer N4
gggagaguauccguugaggcugaacgcguuuacuccgcuaguacgaacccgauugcccagaucggaagagcgucguguagg

SEQ ID NO: 45 - RNA, 81 nt, Artificial
  Synthesized sequence of aptamer N5
gggagaguauccguugaggcugaagccaugcugucaacuuuuaacucgucaccgcucgagaucggaagagcgucguguagg

SEQ ID NO: 46 - RNA, 81 nt, Artificial
  Synthesized sequence of aptamer N15
gggagaguauccguugaggcugaaucggccacagucgacaacuuugaaauccaccugcagaucggaagagcgucguguagg

SEQ ID NO: 47 - RNA, 81 nt, Artificial
  Synthesized sequence of aptamer N20
gggagaguauccguugaggcugagcagcacaugaccucaccuuuuacucugcgcugcaagaucggaagagcgucguguagg

SEQ ID NO: 48 - RNA, 81 nt, Artificial
  Synthesized sequence of aptamer N37
gggagaguauccguugaggcugaucaguccagcaccaacgccguuugcucucgacuacagaucggaagagcgucguguagg

SEQ ID NO: 49 - RNA, 81 nt, Artificial
  Synthesized sequence of aptamer N62
gggagaguauccguugaggcugaguuuccgaacggcccaacuuugaaauccccgcccgagaucggaagagcgucguguagg

SEQ ID NO: 50 - RNA, 81 nt, Artificial
  Synthesized sequence of aptamer N73
gggagaguauccguugaggcugacaacuugaaaucccaacccugcagccgugucugguagaucggaagagcgucguguagg

SEQ ID NO: 51 - RNA, 81 nt, Artificial
  Synthesized sequence of aptamer N164
gggagaguauccguugaggcugaguuuccgaccacgacccaacguuacugcccaccacagaucggaagagcgucguguagg

SEQ ID NO: 52 - RNA, 96 nt, Artificial
  Synthesized sequence of aptamer 4F01
gggagaguauccguuggaggcauagcagcacacgaccagugugccccacacccagugguggucuguggugugcagaucggaagagcgucguguagg

SEQ ID NO: 53 - RNA, 96 nt, Artificial
  Synthesized sequence of aptamer 4F04
gggagaguauccguuggaggcauccguccaccagaccaacguacaaacuccgcugguggucgccuacccuggcagaucggaagagcgucguguagg

SEQ ID NO: 54 - RNA, 96 nt, Artificial
  Synthesized sequence of aptamer 4F05
gggagaguauccguuggaggcaugccgcaggcuacaccacagcuuccccuucagcguguuguggauacucggcagaucggaagagcgucguguagg

SEQ ID NO: 55 - RNA, 96 nt, Artificial
  Synthesized sequence of aptamer R01
gggagaguauccguuggaggcauugccgcacuacagcuuggucugcauauccucugcgcacagcuccaugugcagaucggaagagcgucguguagg

SEQ ID NO: 56 - RNA, 96 nt, Artificial
  Synthesized sequence of aptamer R02
gggagaguauccguuggaggcaugcacgauguccaugaccaacuccagucacggcccugcagcguuaggcuguagaucggaagagcgucguguagg

SEQ ID NO: 57 - RNA, 96 nt, Artificial
  Synthesized sequence of aptamer R03
gggagaguauccguuggaggcauccagcaucaccagcggcacgacgucggacggcuggcugguccgucaccguagaucggaagagcgucguguagg

SEQ ID NO: 58 - RNA, 96 nt, Artificial
  Synthesized sequence of aptamer R04
gggagaguauccguuggaggcaugcagagcuacacggugcaaguagcacguccugccaugcaugcagugcugcagaucggaagagcgucguguagg

SEQ ID NO: 59 - RNA, 96 nt, Artificial
  Synthesized sequence of aptamer R05
gggagaguauccguuggaggcaucgggaagcagcacgacggccucaaugcacuugccgguugguuucggcugcagaucggaagagcgucguguagg

SEQ ID NO: 60 - RNA, 96 nt, Artificial
  Synthesized sequence of aptamer R84
gggagaguauccguuggaggcaucugcgaagugcccaagaccauauccacugcacacgacagcugaugguggcagaucggaagagcgucguguagg

SEQ ID NO: 61 - RNA, 96 nt, Artificial
  Synthesized sequence of aptamer P91
gggagaguauccguuggaggcaugcuacacacacaucgcacgucacccacuauggggacaucuugcggcgugcagaucggaagagcgucguguagg

SEQ ID NO: 62 - RNA, 96 nt, Artificial
  Synthesized Sequence random aptamer Scr-1
gggagaguauccguuggaggcauaucguucagcgugggaucugcuacaacuccugagugcuacauguacgagaagaucggaagagcgucguguagg

SEQ ID NO: 63 - RNA, 81 nt, Artificial
  Synthesized Sequence random aptamer Scr-2
gggagaguauccguugaggcugauucaacaccguuugacguucuugguaucggaagacagaucggaagagcgucguguagg

SEQ ID NO: 64 - DNA, 58 bp, Artificial
  Synthesized Sequence random DNA aptamer SCRAMBLE
aatgatacggcgaccaccgagatctacactctttccctacacgacgctcttccgatct

SEQ ID NO: 65 - DNA, 66 bp, Artificial
  Synthesized Sequence prior art DNA aptamer M5-15
atagtcccatcattcattgtatggtacggcgcggtggcgggtgcgtggagatattagcaagtgtca

SEQ ID NO: 66 - DNA, 66 bp, Artificial
  Synthesized Sequence prior art DNA aptamer T-SO508
atactgccattcatttcatttgcctgtggtgttggggcgggtgcgtttagatatcagcatgtgtca

SEQ ID NO: 67 - DNA, 58 bp, Artificial
  Synthesized Sequence prior art DNA aptamer F5R1
atcgagtgtgtacggggtccggtagggtggcgaggtcttcctgtcgtagcaggatcca

SEQ ID NO: 68 - DNA, 58 bp, Artificial
  Synthesized Sequence prior art DNA aptamer F5R2
atcgagtggacgagtgcctccggtacgagctgtctgatgggtttgcgcgcaggatcca

SEQ ID NO: 69 - DNA, 76 bp, Artificial
  Synthesized Sequence prior art DNA aptamer Tau 3146
gcctgttgtgagcctcctgtcgaacctttggggtggcttgacgaagaaagtagttgagcgtttattcttgtctccc